(19) United States
(12) Patent Application Publication
(10) Pub. No.: US 2026/0169380 A1
(43) Pub. Date: Jun. 18, 2026

(54) RESIST COMPOSITION, LAMINATE, AND PATTERNING PROCESS

(71) Applicant: SHIN-ETSU CHEMICAL CO., LTD., Tokyo (JP)

(72) Inventors: Shun KIKUCHI, Joetsu-shi (JP); Kousuke Ohyama, Joet-su-shi (JP); Ryo Nishikawa, Joetsu-shi (JP); Satoshi Kusama, Joet-shi (JP); Masaki Ohashi, Joetsu-shi (JP)

(73) Assignee: SHIN-ETSU CHEMICAL CO., LTD., Tokyo (JP)

(21) Appl. No.: 19/413,140

(22) Filed: Dec. 9, 2025

(30) Foreign Application Priority Data
Dec. 13, 2024 (JP) 2024-218790

(51) Int. Cl. G03F 7/004; C07D 213/89; C07D 231/16; C07D 333/28; C09D 133/02; G03F 7/00; G03F 7/038; G03F 7/039; G03F 7/09

(52) U.S. Cl. CPC G03F 7/0045; C07D 213/89; C07D 231/16; C07D 333/28; C09D 133/02; G03F 7/0388; G03F 7/039; G03F 7/094; G03F 7/70033

(57) ABSTRACT

The present invention is a resist composition containing: at least one kind of hypervalent iodine compound selected from the following formulae (1), (2), and (3); a carboxy-group-containing compound; and a solvent. This can provide: a non-chemically amplified resist composition excellent in sensitivity and limiting resolution in photolithography using a high-energy beam, especially EB lithography and EUV lithography; a laminate including the resist composition; and a patterning process using the resist composition.

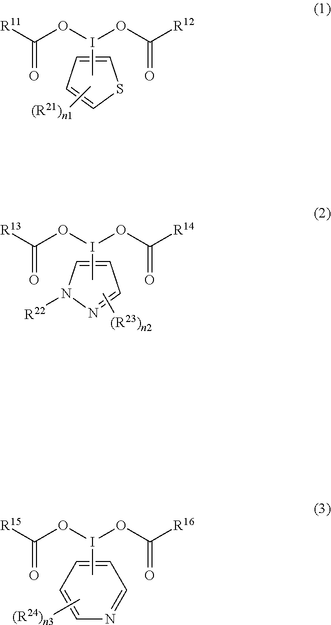

### TECHNICAL FIELD

The present invention relates to: a resist composition; a laminate; and a patterning process using the resist composition.

### BACKGROUND ART

While a higher integration density, higher operating speed and lower power consumption of LSIs are demanded to comply with the expanding IoT market, the effort to reduce the pattern rule is in rapid progress. In particular, logic devices drive forward the miniaturization technology. As the advanced miniaturization technology, devices of 10-nm node are manufactured in a mass scale by the double, triple or quadro-patterning version of the immersion ArF lithography. Furthermore, the experimental mass-scale manufacture of 7-nm node devices by the next-generation extreme ultraviolet ray (EUV) lithography of wavelength 13.5 nm has started.

As miniaturization advances, image blurs due to acid diffusion are regarded as a problem (Non Patent Document 1). In order to ensure resolution for fine patterns with a post-45 nm processed size, it is suggested that not only the enhancement of dissolution contrast, which has been proposed previously, but also the controlling of acid diffusion is important (Non Patent Document 2). In chemically amplified resist compositions, however, the sensitivity and the contrast are enhanced by acid diffusion. Accordingly, an attempt to minimize acid diffusion by lowering the temperature of post-exposure baking (PEB) and shortening the PEB time lowers the sensitivity and contrast markedly.

It is effective to control the acid diffusion by adding an acid generator that generates a bulky acid. Accordingly, it has been proposed to copolymerize a polymer with an acid generator in the form of an onium salt having polymerizable olefin. In post-16 nm processed size patterning of resist films, however, it is considered that patterning is impossible with chemically amplified resist compositions in view of the acid diffusion. Accordingly, development of a non-chemically amplified resist composition is desired.

Examples of materials for a non-chemically amplified resist composition include polymethyl methacrylate (PMMA). PMMA is a positive resist material whose solubility in an organic solvent developer increases due to decreased molecular weight caused by scission of the main chain by EUV irradiation.

Hydrogensilsesquioxane (HSQ) is a negative resist material which turns insoluble in an alkaline developer through crosslinking by condensation reaction of silanol generated by EUV irradiation. Calixarene substituted with chlorine also functions as a negative resist material. These negative resist materials have a small molecular size prior to crosslinking and are free from causing blurs due to acid diffusion, and therefore, exhibit smaller edge roughness and very high resolution. Accordingly, the materials have been used as a pattern transfer material to show the resolution limit of the exposure apparatus. These materials, however, are insufficient in sensitivity, and further improvement is required.

The number of photons in EUV exposure being small is a factor that causes difficulties in developing materials for EUV lithography. The energy of EUV is much higher than that of an ArF excimer laser beam, and the number of photons in EUV exposure is 1/14 of that of ArF exposure. Furthermore, the size of the pattern formed by EUV exposure is half of that in ArF exposure or less. Therefore, EUV exposure is easily affected by variation in the number of photons. The variation in the number of photons in a radiation light region of extremely short wavelengths is the physical phenomenon of shot noise, and it is impossible to eliminate the influence of the variation. Therefore, so-called probability theory (stochastics) is attracting attention. The influence of shot noise cannot be eliminated, but there is discussion of how to reduce this influence. Due to the influence of shot noise, not only are critical dimension uniformity (CDU) and line width roughness (LWR) increased, a phenomenon that a hole gets blocked at a probability of one to several millions is observed. If a hole gets blocked, conduction failure occurs and the transistor does not function, and the performance of the entire device is adversely affected. Considering sensitivity in practical terms, resist compositions that mainly contain PMMA or HSQ are greatly affected by stochastics, and cannot achieve the desired resolution performance.

The introduction of an element that greatly absorbs EUV light is attracting attention as a means for reducing the influence of shot noise on the side of the resist. Patent Document 1 proposes a chemically amplified resist composition containing iodine atoms, which greatly absorb EUV light. However, as stated above, a chemically amplified resist composition cannot realize excellent resolution performance in EUV lithography, in which the processed size is to be further miniaturized in the future. Especially in a line-and-space pattern, pattern collapse and the breaking of a line increase remarkably as the pattern size is reduced, and therefore, reducing these leads to the improvement of limiting resolution.

Patent Document 2 proposes a negative resist composition containing a tin compound. This composition mainly contains the element tin, which greatly absorbs EUV light, and therefore, stochastics is improved, and high sensitivity and high resolution can be realized. However, such a so-called metal resist has many problems such as insufficient solubility in a solvent for resists, storage stability, and defects due to residues after etching. Furthermore, a metal resist is a negative resist in which mainly the exposed portions form a metal oxide and become insoluble in a developer. Therefore, when the resist is applied to the patterning of contact holes, an additional reversal process step is necessary, and there are also concerns regarding costs.

### CITATION LIST

### Patent Literature

Patent Document 1: JP2018-5224A
Patent Document 2: JP2021-503482A

### Non Patent Literature

Non Patent Document 1: SPIE Vol. 5039 p 1 (2003)
Non Patent Document 2: SPIE Vol. 6520 p 65203L-1 (2007)

### SUMMARY OF INVENTION

### Technical Problem

The present invention has been made in view of the above-described circumstances, and an object thereof is to provide: a non-chemically amplified resist composition excellent in sensitivity and limiting resolution in photolithography using a high-energy beam, especially electron beam (EB) lithography and EUV lithography; a laminate including the resist composition; and a patterning process using the resist composition.

### Solution to Problem

To achieve the object, the present invention provides a resist composition comprising: at least one kind of hypervalent iodine compound selected from the following formulae (1), (2), and (3); a carboxy-group-containing compound; and a solvent,

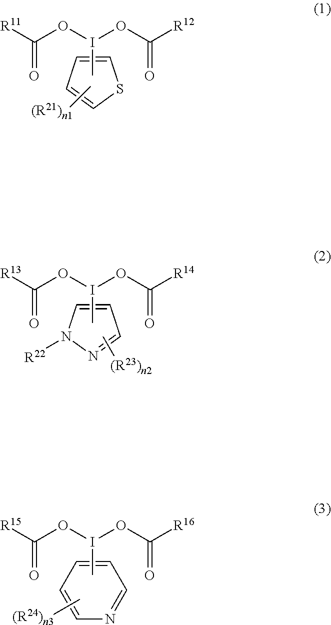

wherein “n1” represents 0, 1, 2, or 3, “n2” represents 0, 1, or 2, “n3” represents 0, 1, 2, 3, or 4, R¹¹ to R¹⁶ each independently represent a halogen atom or a hydrocarbyl group having 1 to 10 carbon atoms and optionally containing a heteroatom, and R²¹ to R²⁴ each independently represent a halogen atom or a hydrocarbyl group having 1 to 40 carbon atoms and optionally containing a heteroatom, when “n1” is 2 or more, the R²¹s are identical to or different from each other and the R²¹s may be bonded to each other to form a ring together with the carbon atoms of the heteroaromatic ring bonded thereto, when “n2” is 2, the R²³s are identical to or different from each other and the R²³s may be bonded to each other to form a ring together with the carbon atoms of the heteroaromatic ring bonded thereto, and when “n3” is 2 or more, the R²⁴s are identical to or different from each other and the R²⁴s may be bonded to each other to form a ring together with the carbon atoms of the heteroaromatic ring bonded thereto.

The inventive resist composition as described is excellent in sensitivity and limiting resolution in photolithography using a high-energy beam, especially EB lithography and EUV lithography.

In this case, the carboxy-group-containing compound is preferably one or both of a polymer including a repeating unit represented by the following formula (4) and a compound represented by the following formula (5),

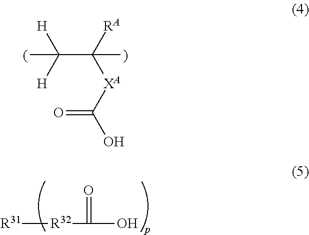

wherein R^{A} represents a hydrogen atom, a halogen atom, a methyl group, or a trifluoromethyl group; X^{A} represents a single bond, a phenylene group, a naphthylene group, or *—C(═O)—O—X^{A1}—; X^{A1} represents a saturated hydrocarbylene group having 1 to 10 carbon atoms, a phenylene group, or a naphthylene group, the saturated hydrocarbylene group optionally containing a hydroxy group, an ether bond, an ester bond, or a lactone ring; “*” represents an attachment point to the carbon atom of the main chain; “p” represents 1, 2, 3, or 4; R³¹ represents a p-valent hydrocarbon group having 1 to 40 carbon atoms or a p-valent heterocyclic group having 2 to 40 carbon atoms, when “p” is 2, the R³¹ may be an ether bond, a carbonyl group, an azo group, a thioether bond, a carbonate bond, a carbamate bond, a sulfinyl group, or a sulfonyl group, wherein part or all of hydrogen atoms of the p-valent hydrocarbon group or the p-valent heterocyclic group may be substituted with a group containing a heteroatom, and part of —CH₂— of the p-valent hydrocarbon group may be substituted with a group containing a heteroatom; and R³² represents a single bond or a hydrocarbylene group having 1 to 20 carbon atoms, part or all of hydrogen atoms of the hydrocarbylene group may be substituted with a group containing a heteroatom, part of —CH₂— of the hydrocarbylene group may be substituted with a group containing a heteroatom, and when “p” is 2, 3, or 4, the R³²s are identical to or different from each other.

As the carboxy-group-containing compound contained in the inventive resist composition, such a polymer or monomolecular compound is preferable.

The present invention also provides a laminate comprising: a substrate; and a resist film, which is a film body formed of the above-described resist composition, on the substrate.

In a laminate including a resist film obtained from the inventive resist composition, the resist film, which is a film body of the above-described resist composition, has high sensitivity, also exhibits excellent limiting resolution, is effective for precise fine processing, and in addition, can be applied to either positive or negative patterning. Therefore, the laminate has a wide range of uses, and is highly useful in resist process technology.

In this case, the laminate can further comprise a resist underlayer film between the substrate and the resist film. In addition, the resist film preferably contains a product made by a ligand exchange reaction of the hypervalent iodine compound and the carboxy-group-containing compound.

The inventive laminate can be as described above in accordance with requirements.

The present invention also provides a patterning process comprising the steps of:
forming a resist film by using the above-described resist composition on a substrate or on a resist underlayer film of a substrate on which the resist underlayer film has been laminated;
exposing the resist film by using a high-energy beam; and
developing the exposed resist film by using a developer.

In the inventive patterning process, a resist composition that is excellent in sensitivity and resolution in photolithography using a high-energy beam, especially electron beam (EB) lithography and EUV lithography, is used, and therefore, the patterning process is useful for finer patterning.

In this case, the high-energy beam is preferably an i-line, a KrF excimer laser beam, an ArF excimer laser beam, an electron beam, or an extreme ultraviolet ray.

In the inventive patterning process, using such a high-energy beam makes finer patterning possible.

In the inventive patterning process, the developer may dissolve exposed portions and not dissolve unexposed portions, or may dissolve unexposed portions and not dissolve exposed portions.

According to the inventive patterning process, a positive or negative pattern can be formed by selecting the developer appropriately, and therefore, the patterning process can be widely applied to various kinds of fine patterning.

### Advantageous Effects of Invention

The inventive resist composition can achieve both high sensitivity and high resolution, particularly in photolithography using an i-line, a KrF excimer laser beam, an ArF excimer laser beam, an EB, or an EUV, and is extremely useful on forming a fine pattern.

### DESCRIPTION OF EMBODIMENTS

To achieve the object, the present inventors have studied earnestly and found out that a resist composition mainly containing a predetermined hypervalent iodine compound and a carboxy-group-containing compound (a polymer or a monomolecular compound) can give a resist film that exhibits excellent sensitivity and resolution and is extremely useful for precise fine processing, and achieved the present invention.

That is, the present invention is a resist composition comprising: a particular hypervalent iodine compound described later; a carboxy-group-containing compound; and a solvent.

Hereinafter, the present invention will be described in detail, but the present invention is not limited thereto. Note that, in the present description, the recitations of numerical ranges by endpoints include all numbers subsumed within that range (e. g. “0 to 3” includes 0, 1, 2, and 3).

### [Resist Composition]

The inventive resist composition mainly contains a predetermined hypervalent iodine compound, a carboxy-group-containing compound, and a solvent.

### [Hypervalent Iodine Compound]

Hypervalent iodine compound is a general term for iodine compounds having valence electrons formally exceeding the octet rule, and examples include tricoordinate iodine compounds (iodine (III) compounds), having an oxidation number of +3, and pentacoordinate iodine compounds (iodine (V) compounds), having an oxidation number of +5.

The hypervalent iodine compound, which is a main component of the inventive resist composition, is at least one kind of tricoordinate hypervalent iodine compound selected from the following formulae (1), (2), and (3).

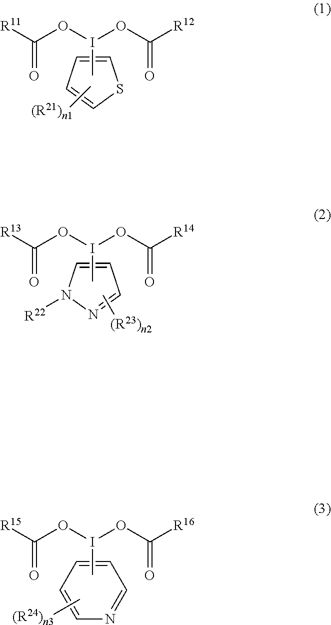

In the formulae, “n1” represents 0, 1, 2, or 3, “n2” represents 0, 1, or 2, “n3” represents 0, 1, 2, 3, or 4, R¹¹ to R¹⁶ each independently represent a halogen atom or a hydrocarbyl group having 1 to 10 carbon atoms and optionally containing a heteroatom, and R²¹ to R²⁴ each independently represent a halogen atom or a hydrocarbyl group having 1 to 40 carbon atoms and optionally containing a heteroatom, when “n1” is 2 or more, the R²¹s are identical to or different from each other and the R²¹s may be bonded to each other to form a ring together with the carbon atoms of the heteroaromatic ring bonded thereto, when “n2” is 2, the R²³s are identical to or different from each other and the R²³s may be bonded to each other to form a ring together with the carbon atoms of the heteroaromatic ring bonded thereto, and when “n3” is 2 or more, the R²⁴s are identical to or different from each other and the R²⁴s may be bonded to each other to form a ring together with the carbon atoms of the heteroaromatic ring bonded thereto.

The inventive resist composition contains a heterocycle-containing hypervalent iodine compound as a hypervalent iodine compound. A heterocycle-containing hypervalent iodine compound has a heterocycle having a substituent hypervalent iodine atom, and in the present invention, the heterocycle contains sulfur or nitrogen. In the formulae (1) to (3), the hypervalent iodine (III) is a substituent in a heteroaromatic ring, and the heteroaromatic ring is thiophene, which is a sulfur-containing heterocycle, in the formula (1), pyrazole, which is a nitrogen-containing heterocycle, in the formula (2), and pyridine, which is a nitrogen-containing heterocycle, in the formula (3). Note that a heteroaromatic ring is an aromatic ring containing at least one heteroatom as part of the aromatic ring.

When a ring in a cyclic compound containing a heteroatom has been aromatized, the heteroaromatic ring can be classified into π-sufficient systems and π-deficient systems based on the π electron density on the carbon atoms. A π-sufficient system is one where the π electron density on each carbon atom is higher than the π electron density of benzene as standard, and a π-deficient system is one where the π electron density is low. According to this classification, thiophene and pyrazole are aromatic rings belonging to a π-sufficient system, and pyridine is an aromatic ring belonging to a π-deficient system.

The hypervalent iodine (III) compounds represented by the formulae (1), (2), and (3) are affected by the π electron density of the heteroaromatic ring to which the iodine (III) is bonded, and therefore, reactivity varies compared to compounds in which iodine (III) is bonded to an aromatic hydrocarbon ring. In addition, the heteroaromatic ring itself also has various affinities to polar/non-polar solvents by virtue of properties, such as the basicity or acidity of the heteroaromatic ring and the ability to form hydrogen bonds, and has a varied solubility.

That is, regarding the hypervalent iodine (III) compounds represented by the formulae (1), (2), and (3), the reactivity and solvent solubility of the hypervalent iodine compound can be adjusted by selecting the thiophene, pyrazole, or pyridine skeleton as the heteroaromatic ring, and more desirable reactivity and solvent solubility can be achieved by one of the hypervalent iodine compounds represented by the formulae (1), (2), and (3), or by a combination of two or more thereof.

As described, heterocycle-containing hypervalent iodine compounds have an advantageous characteristic that it is possible to increase the degree of freedom in adjusting the properties of the resist composition compared to non-heteroaromatic-ring-containing hypervalent iodine compounds; for example, by taking advantage of the properties of the heteroaromatic ring, it is possible to enhance solvent solubility and adjust the reactivity of the hypervalent iodine.

In the formulae, “n1” represents 0, 1, 2, or 3, “n2” represents 0, 1, or 2, and “n3” represents 0, 1, 2, 3, or 4.

R¹¹ to R¹⁶ each independently represent a halogen atom or a hydrocarbyl group having 1 to 10 carbon atoms and optionally containing a heteroatom.

Examples of the halogen atom represented by R¹¹ to R¹⁶ include a fluorine atom, a chlorine atom, a bromine atom, and an iodine atom. The hydrocarbyl group represented by R¹¹ to R¹⁶ having 1 to 10 carbon atoms may be saturated or unsaturated, and may be linear, branched, or cyclic. Specific examples thereof include: alkyl groups having 1 to 10 carbon atoms, such as a methyl group, an ethyl group, a propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 10 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decanyl group, and an adamantyl group; alkenyl groups, such as a vinyl group and an allyl group; aryl groups having 6 to 10 carbon atoms, such as a phenyl group and a naphthyl group; and groups obtained by combining these groups. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl groups may contain a hydroxy group, a cyano group, a halogen atom, a carbonyl group, an ether bond, a thioether bond, an ester bond, a sulfonic acid ester bond, a carbonate bond, a carbamate bond, a lactone ring, a sultone ring, a carboxylic acid anhydride (—C(═O)—O—C(═O)—), etc. As R¹¹ to R¹⁶, hydrocarbyl groups having 1 to 4 carbon atoms are preferable.

R²¹ to R²⁴ each independently represent a halogen atom or a hydrocarbyl group having 1 to 40 carbon atoms and optionally containing a heteroatom, when “n1” is 2 or more, the R²¹s are identical to or different from each other and the R²¹s may be bonded to each other to form a ring together with the carbon atoms of the heteroaromatic ring bonded thereto, when “n2” is 2, the R²³s are identical to or different from each other and the R²³s may be bonded to each other to form a ring together with the carbon atoms of the heteroaromatic ring bonded thereto, and when “n3” is 2 or more, the R²⁴s are identical to or different from each other and the R²⁴s may be bonded to each other to form a ring together with the carbon atoms of the heteroaromatic ring bonded thereto.

Examples of the halogen atom represented by R²¹ to R²⁴ include a fluorine atom, a chlorine atom, a bromine atom, and an iodine atom. The hydrocarbyl group represented by R²¹ to R²⁴ having 1 to 40 carbon atoms may be saturated or unsaturated, and may be linear, branched, or cyclic. Specific examples thereof include: alkyl groups having 1 to 40 carbon atoms, such as a methyl group, an ethyl group, a propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decanyl group, an adamantyl group, and an adamantylmethyl group; and aryl groups having 6 to 40 carbon atoms, such as a phenyl group, a naphthyl group, and an anthracenyl group. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl groups may contain a hydroxy group, a cyano group, a halogen atom, a carbonyl group, an ether bond, a thioether bond, an ester bond, a sulfonic acid ester bond, a carbonate bond, a carbamate bond, a lactone ring, a sultone ring, a carboxylic acid anhydride (—C(═O)—O—C(═O)—), etc. Furthermore, R²¹ to R²⁴ can substitute any position in the heteroaromatic rings in the formulae, but it is preferable to substitute the hydrogen atom bonded to a carbon atom of the heterocycle.

In the following, examples of the hypervalent iodine compound are shown, and in the formulae, the substituents on the heteroaromatic rings can substitute any position (heteroatom or carbon atom) of the rings.

Specific examples of the hypervalent iodine compound represented by the formula (1) include the following, but are not limited thereto.

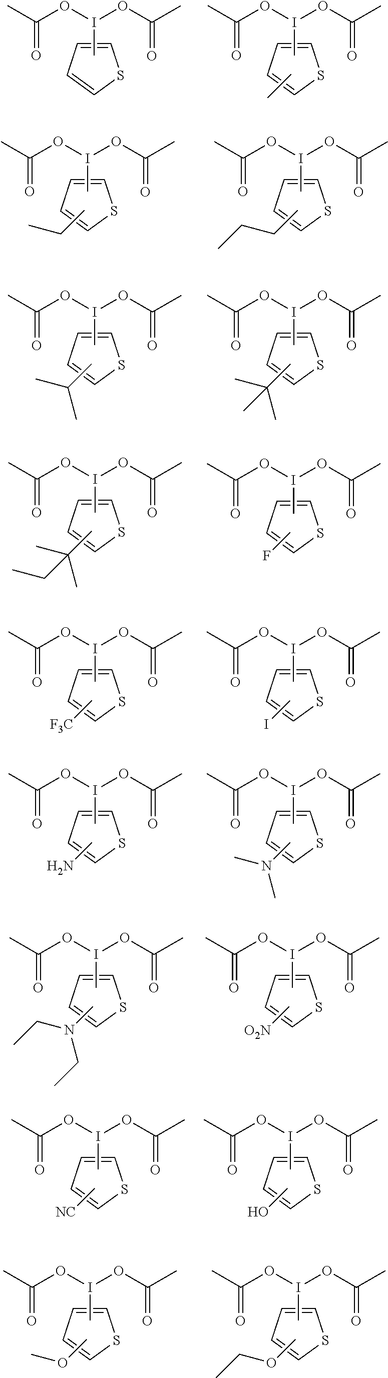

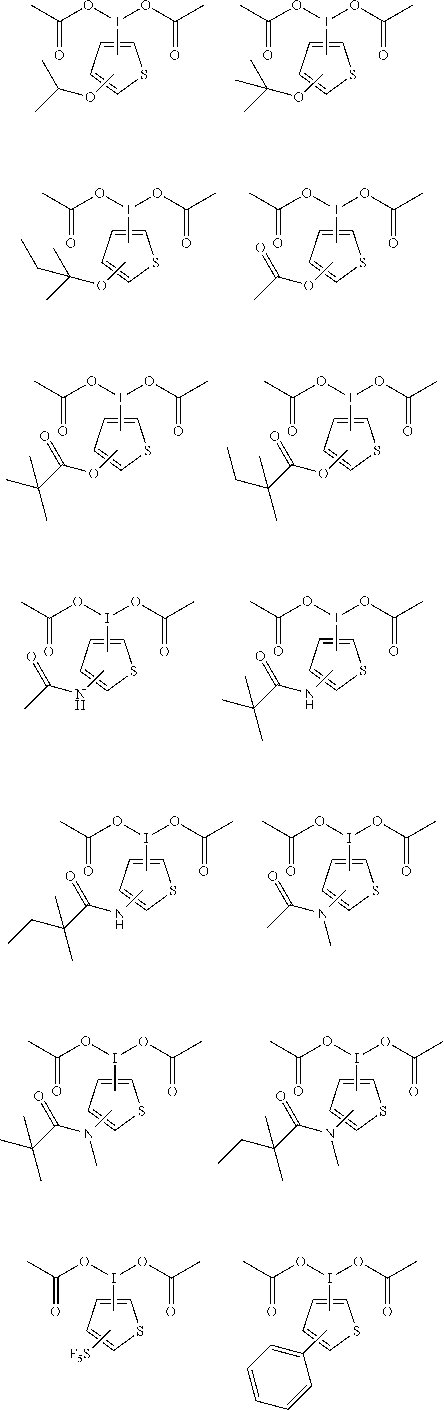

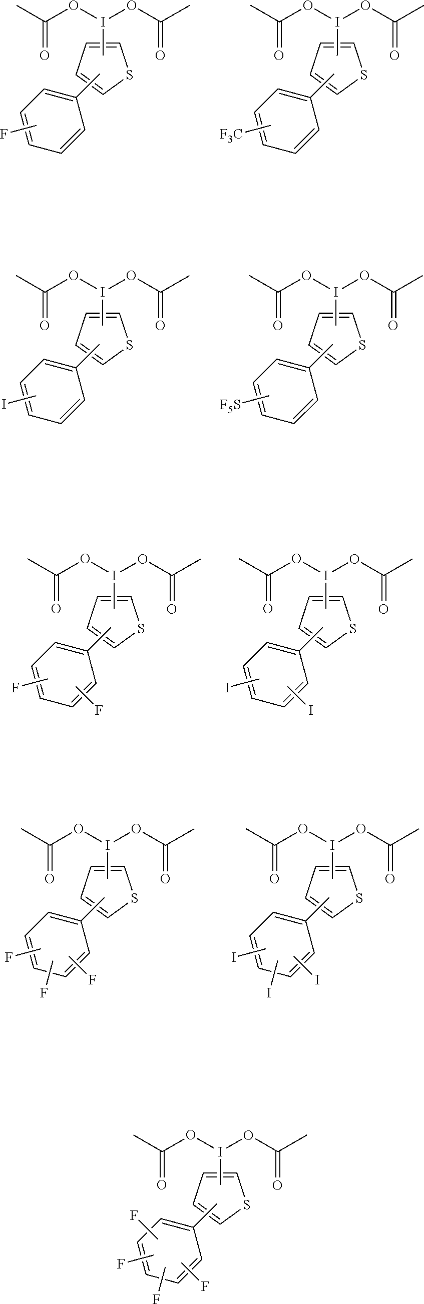

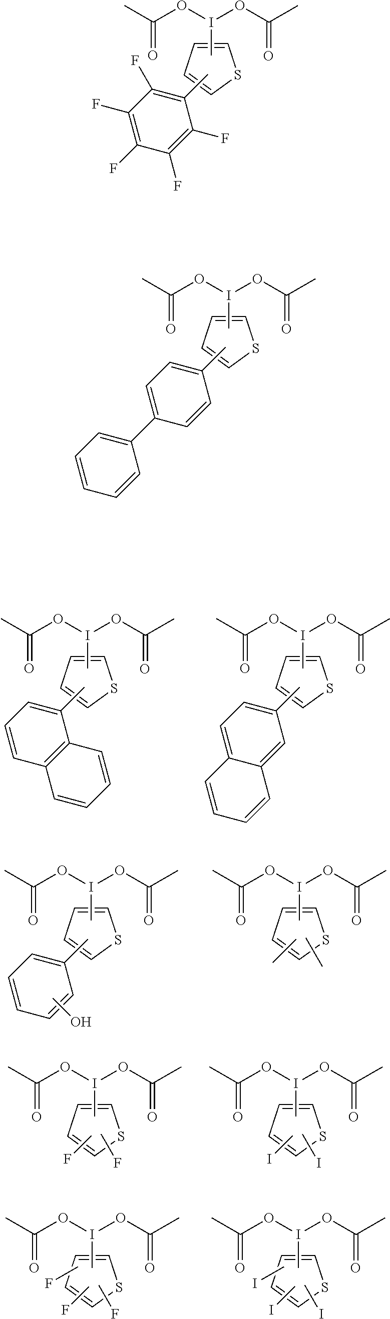

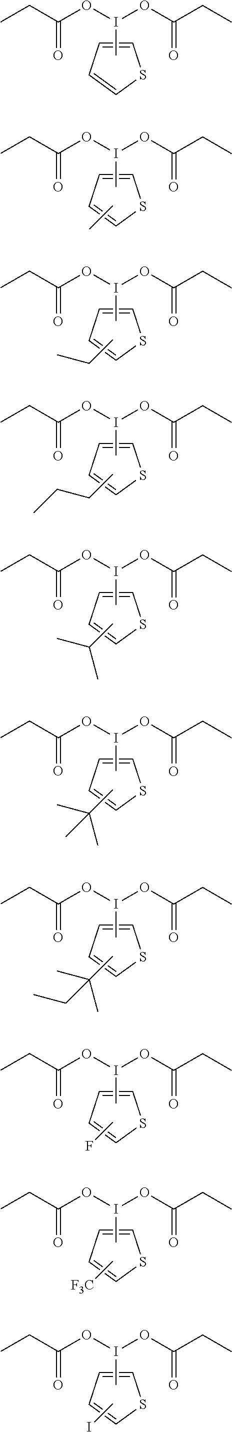

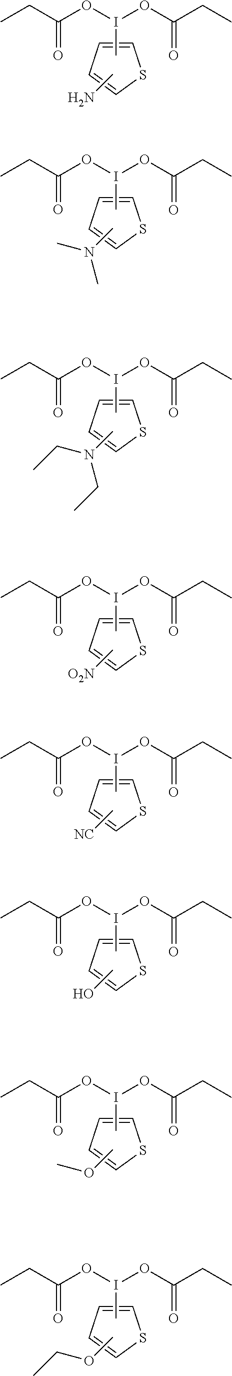

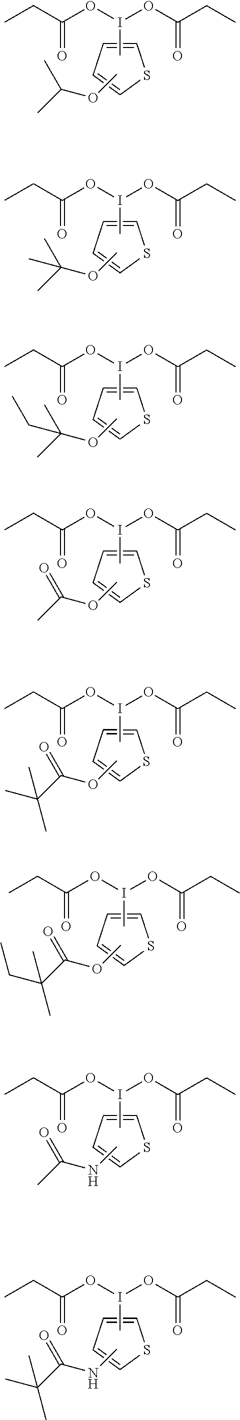

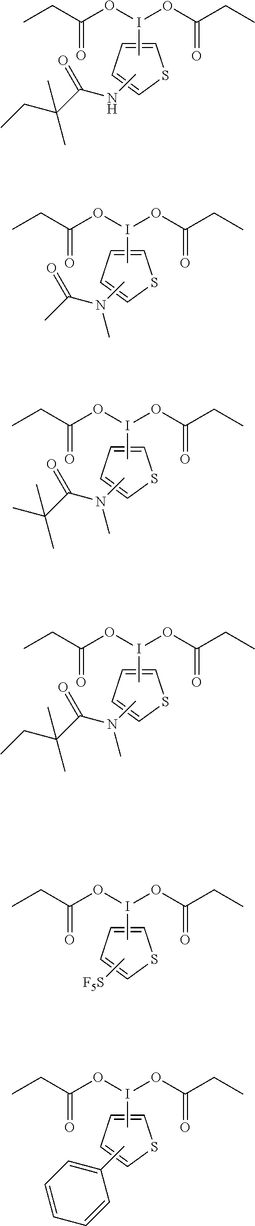

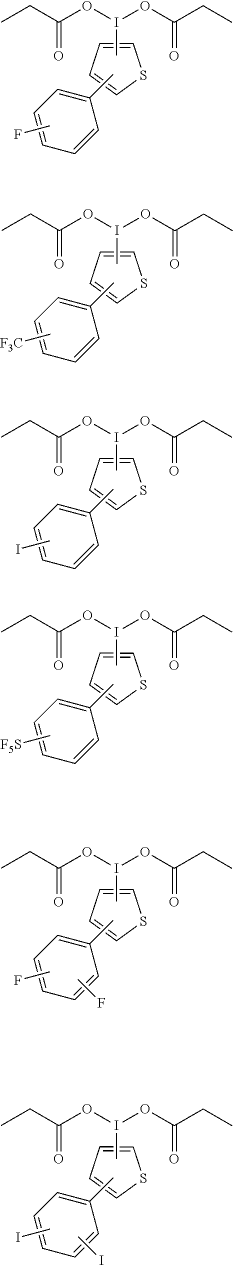

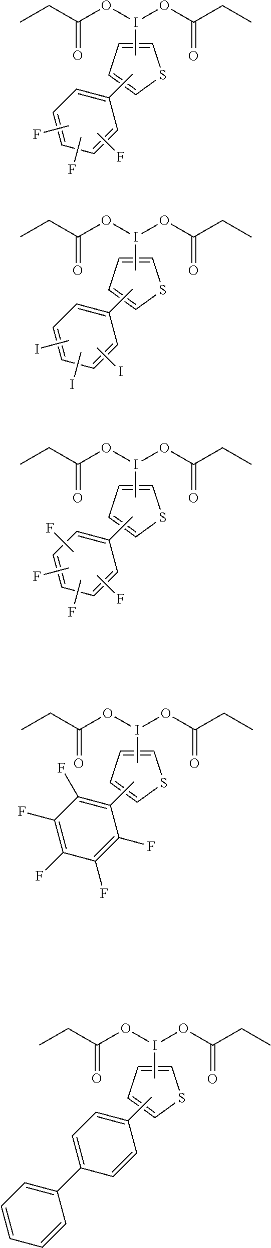

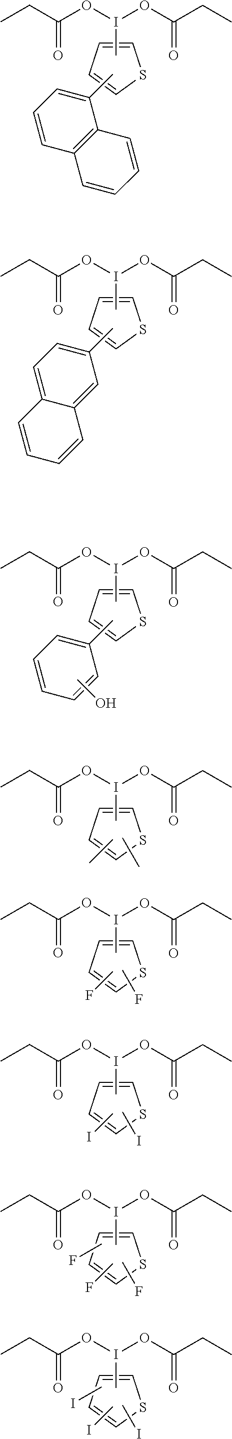

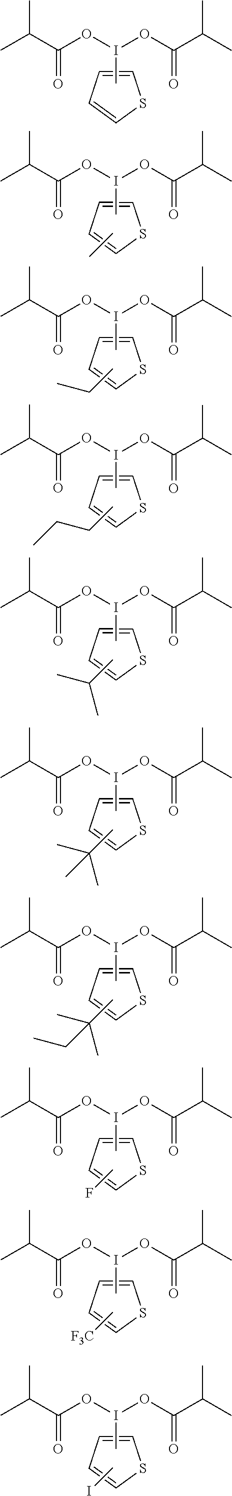

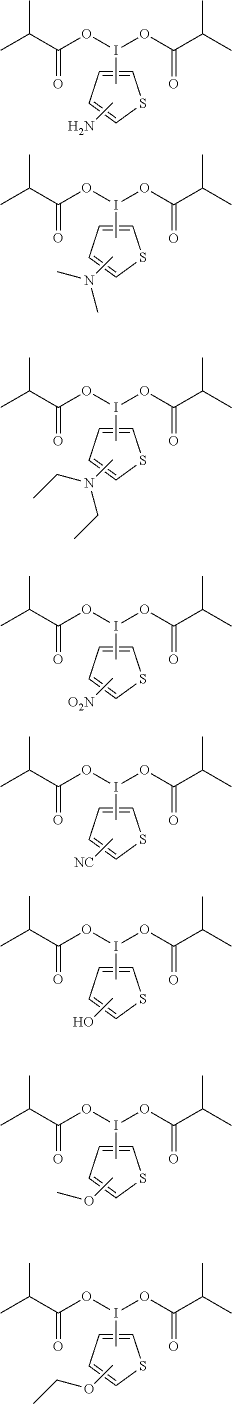

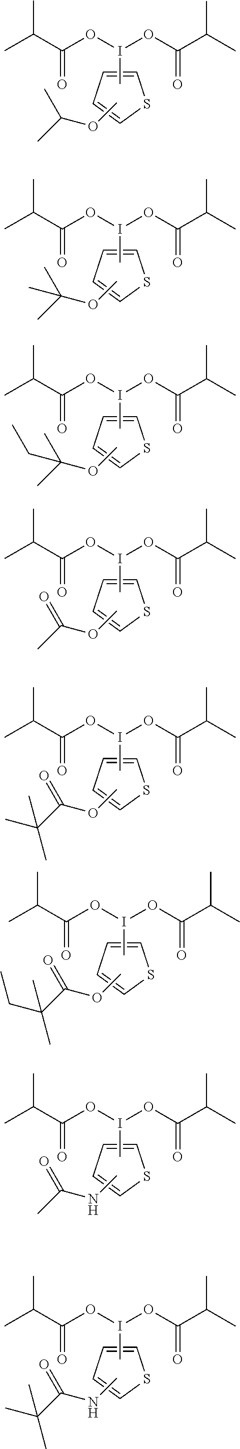

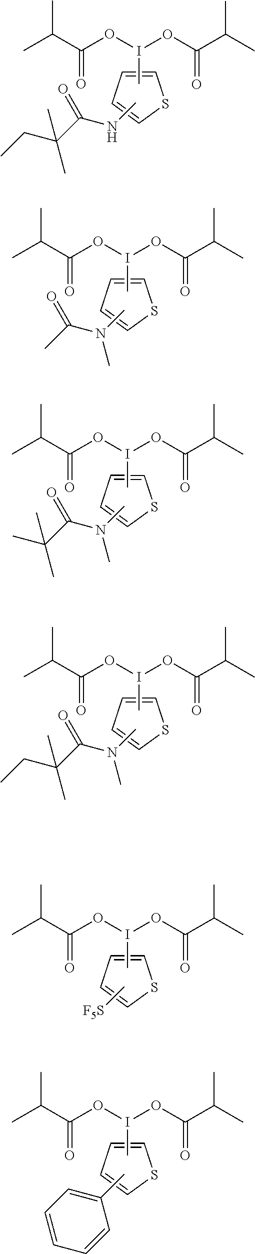

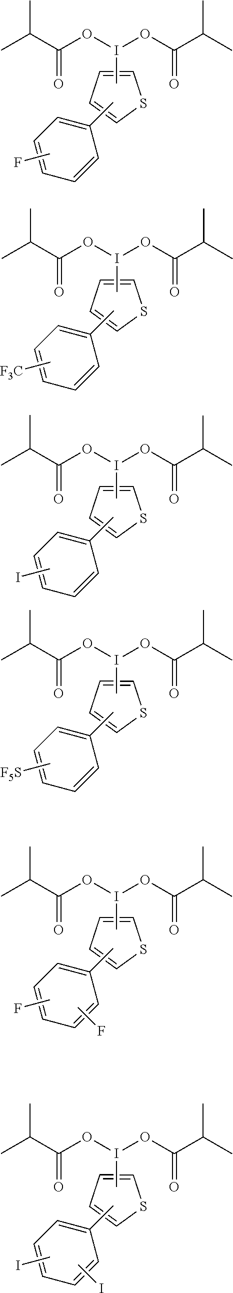

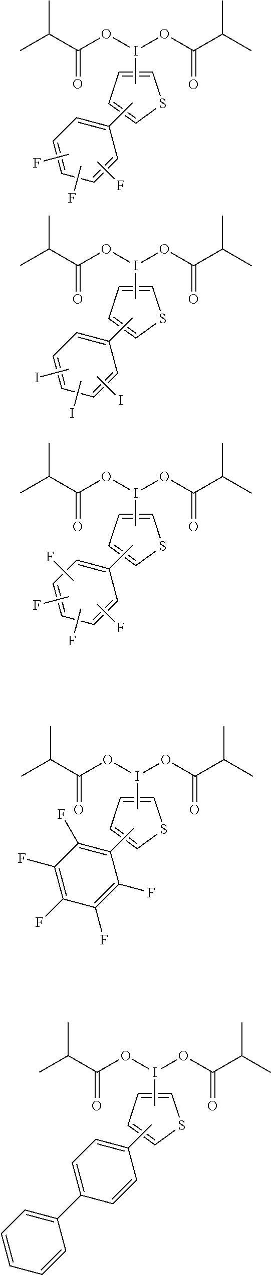

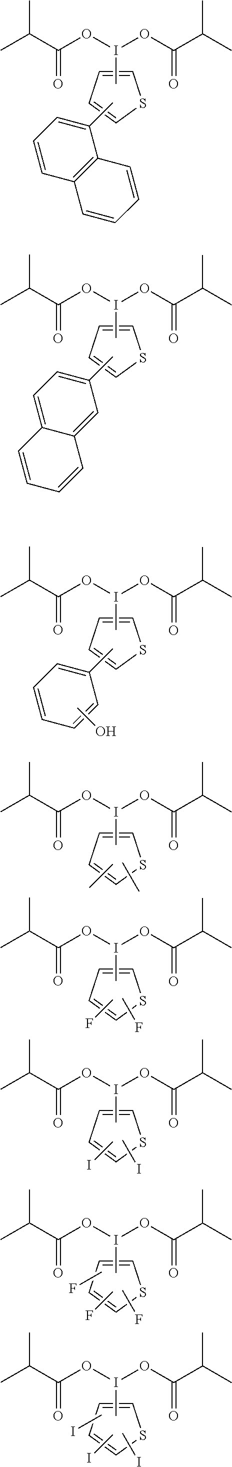

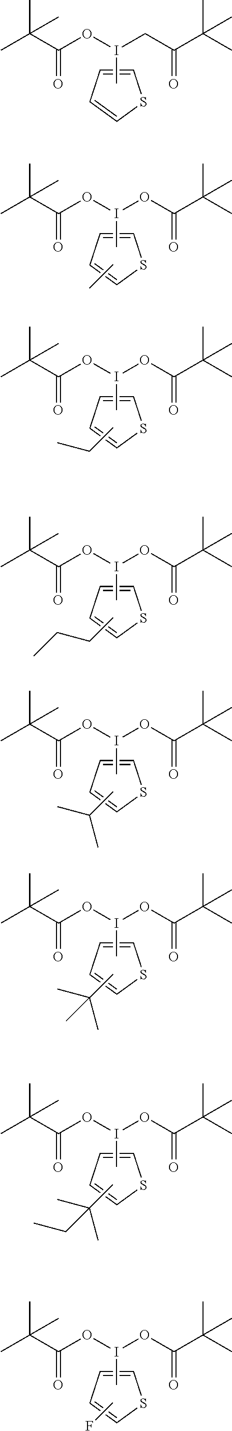

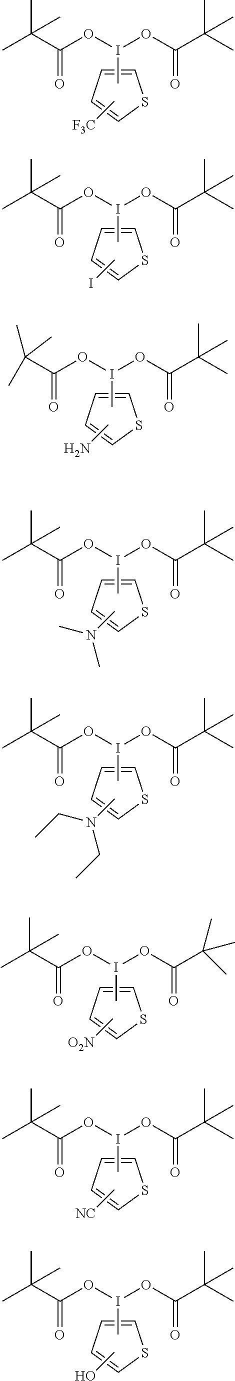

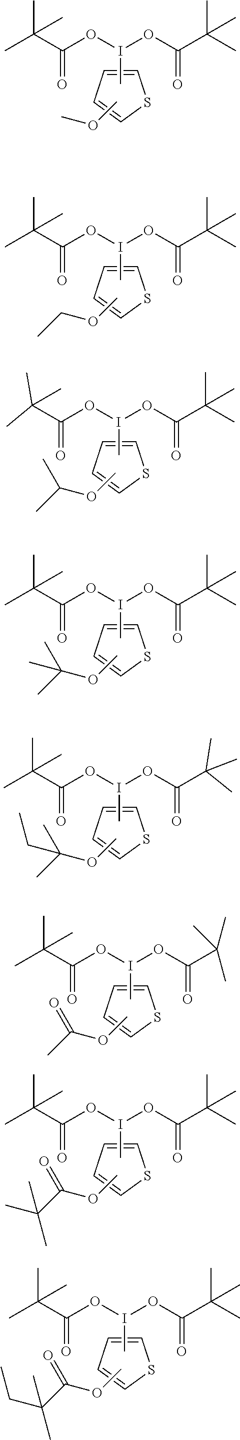

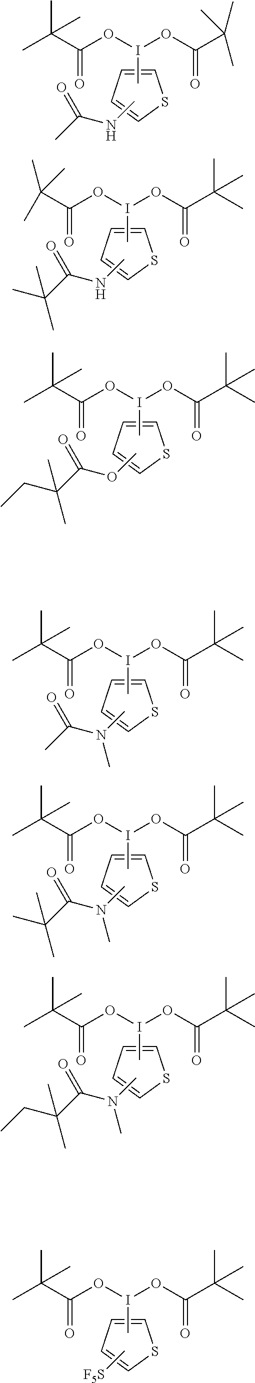

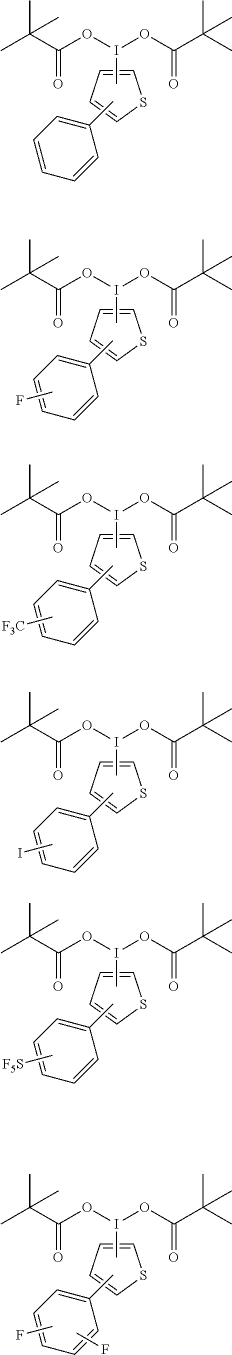

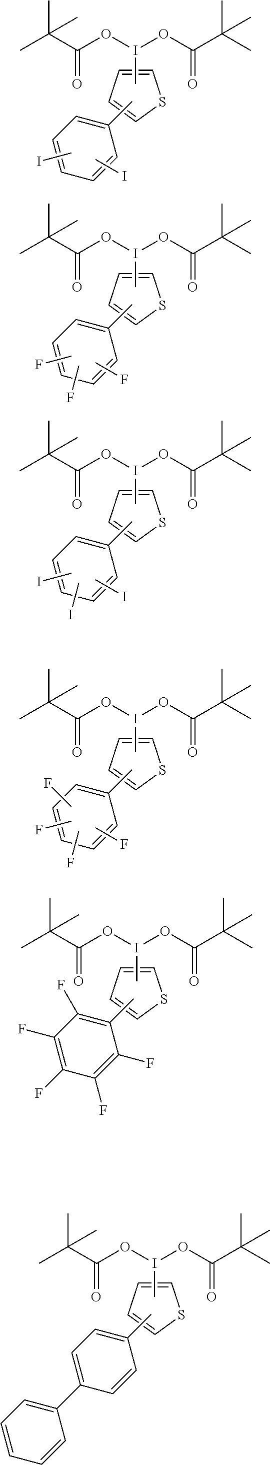

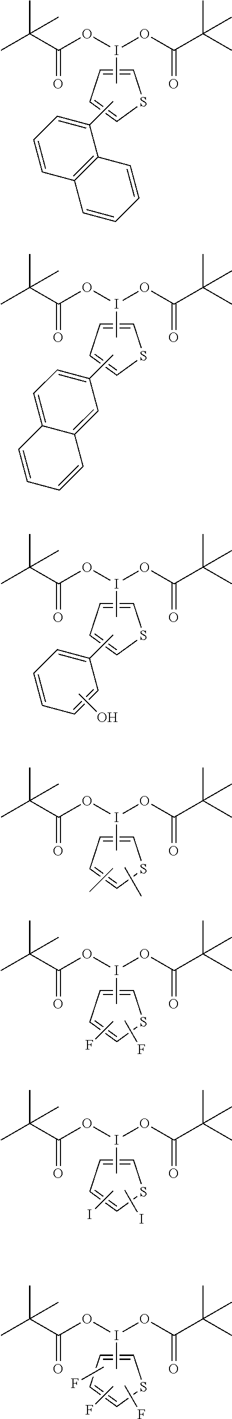

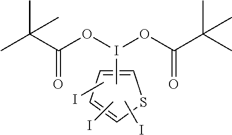

Specific examples of the hypervalent iodine compound represented by the formula (2) include the following, but are not limited thereto.

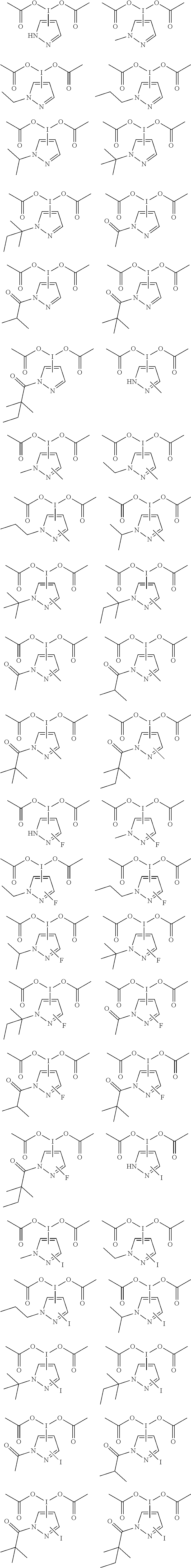

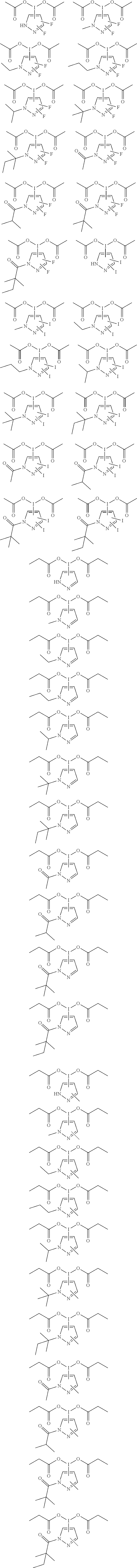

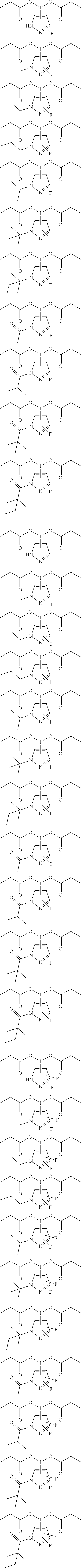

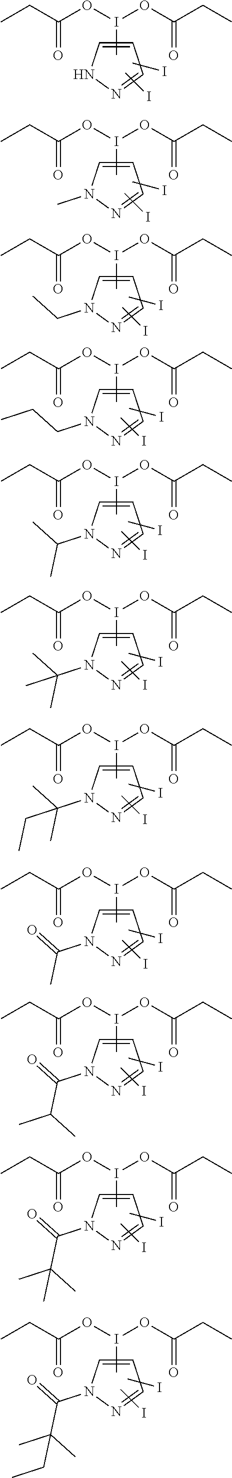

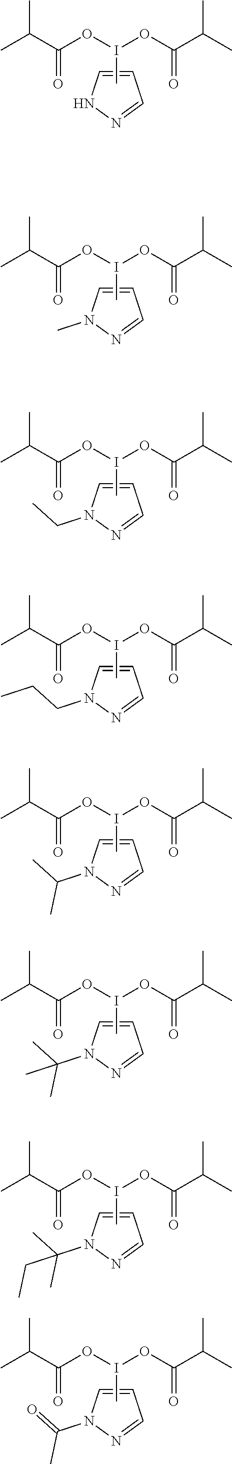

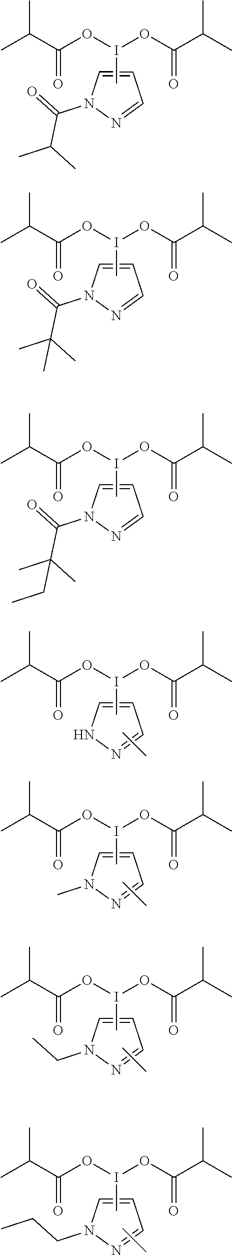

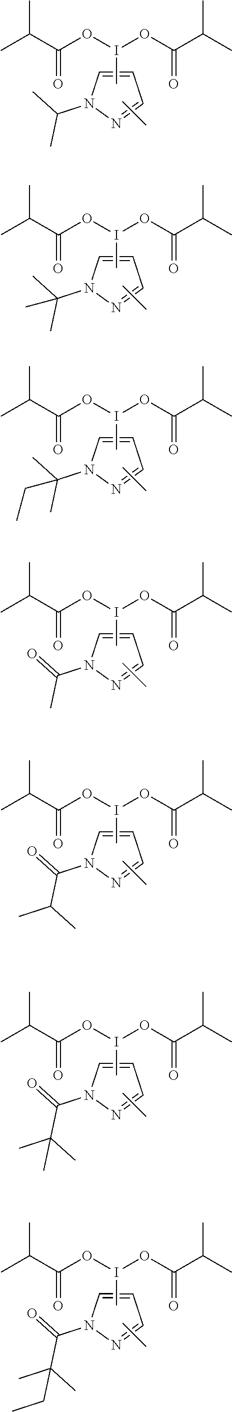

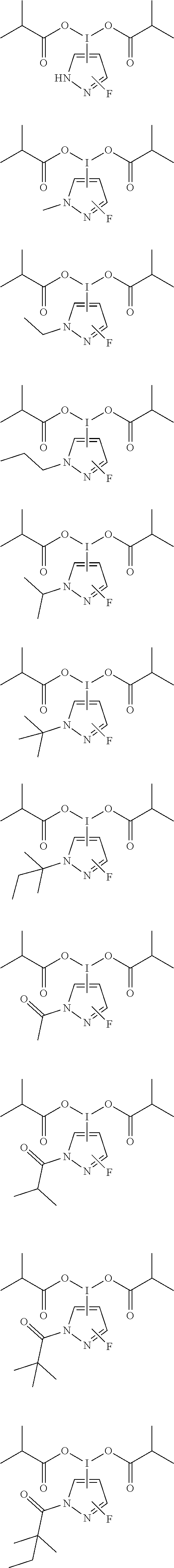

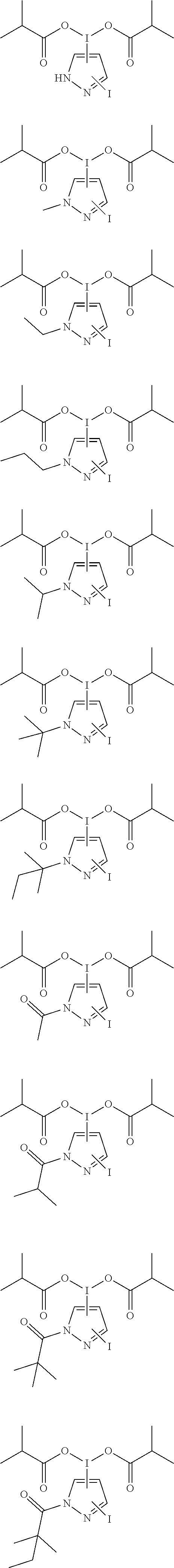

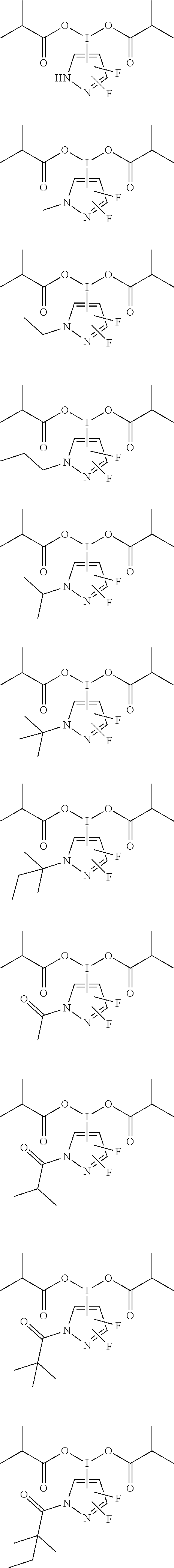

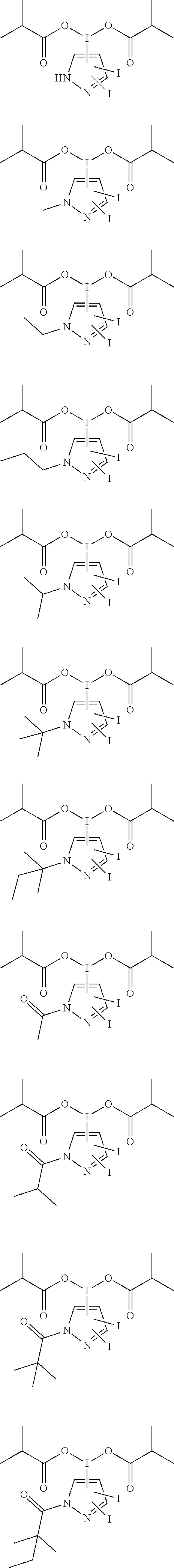

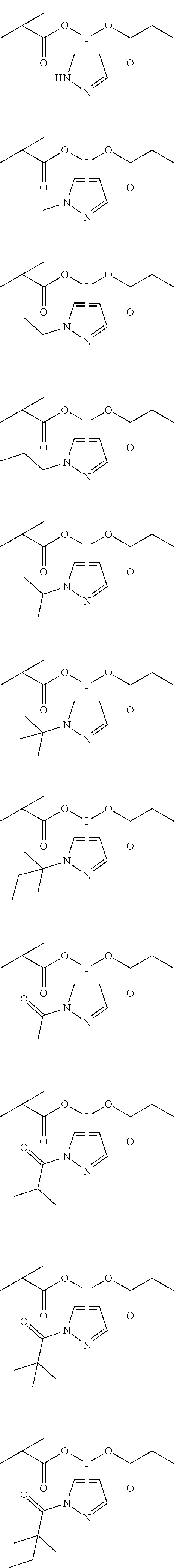

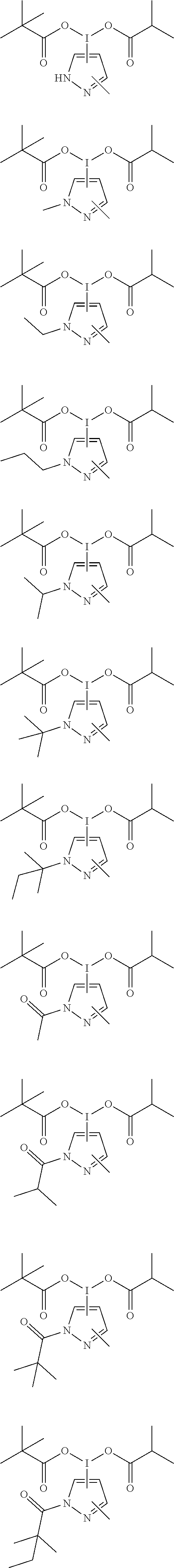

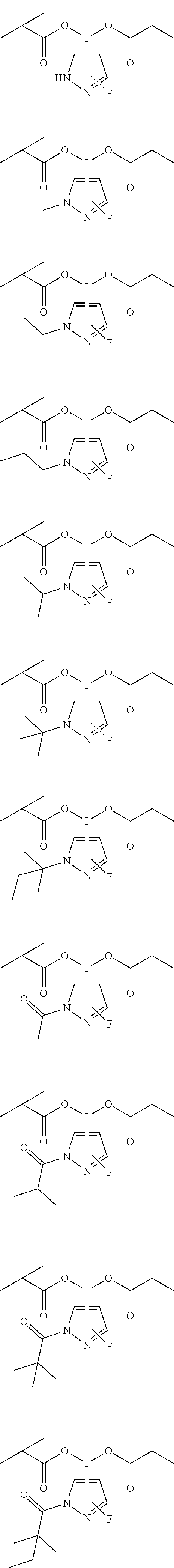

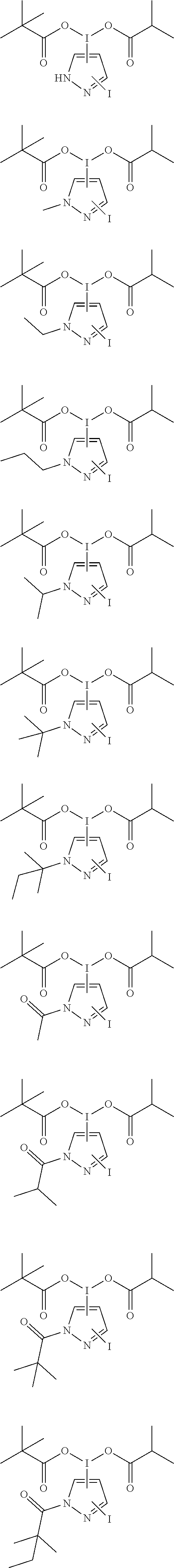

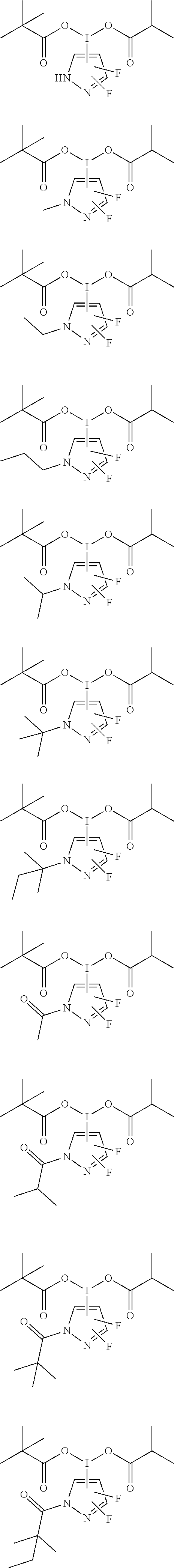

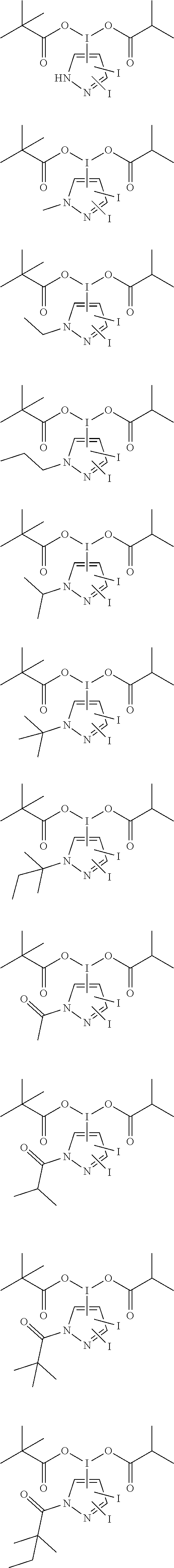

Specific examples of the hypervalent iodine compound represented by the formula (3) include the following, but are not limited thereto.

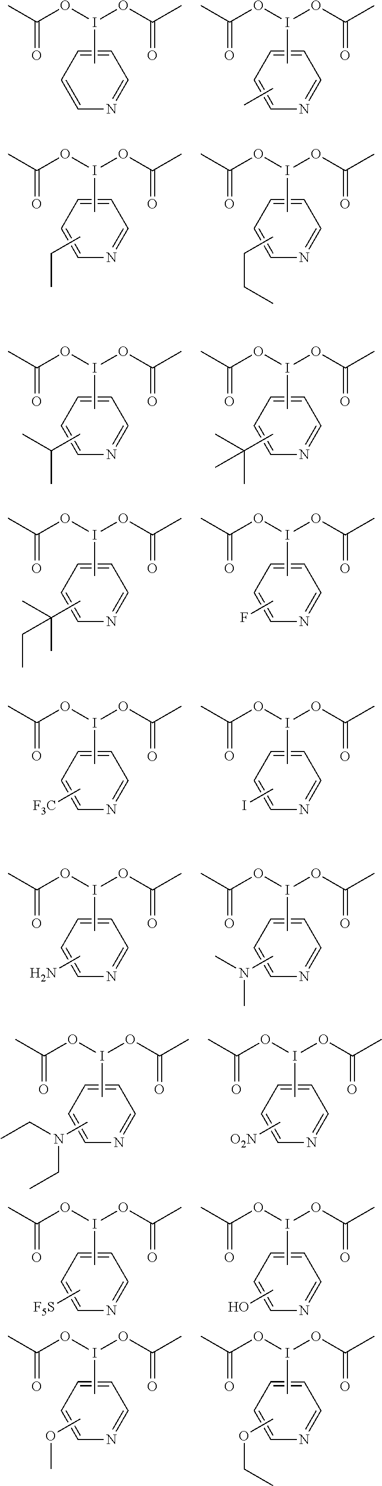

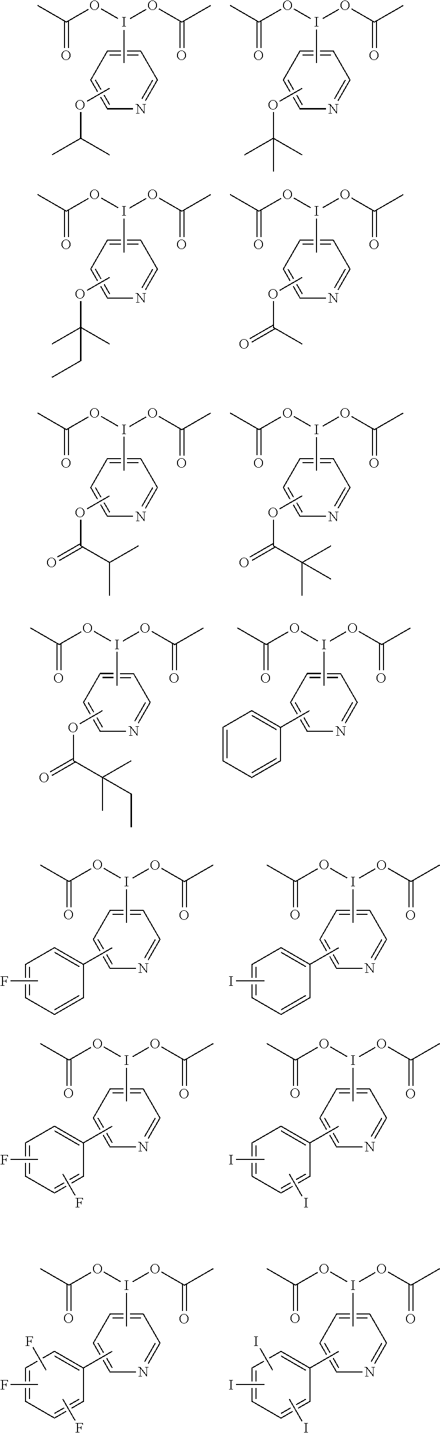

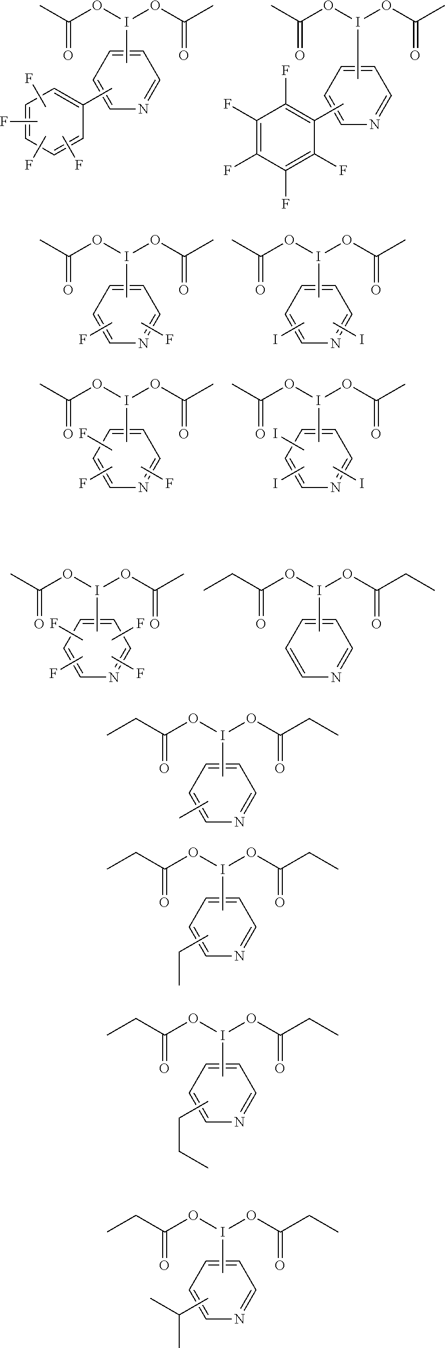

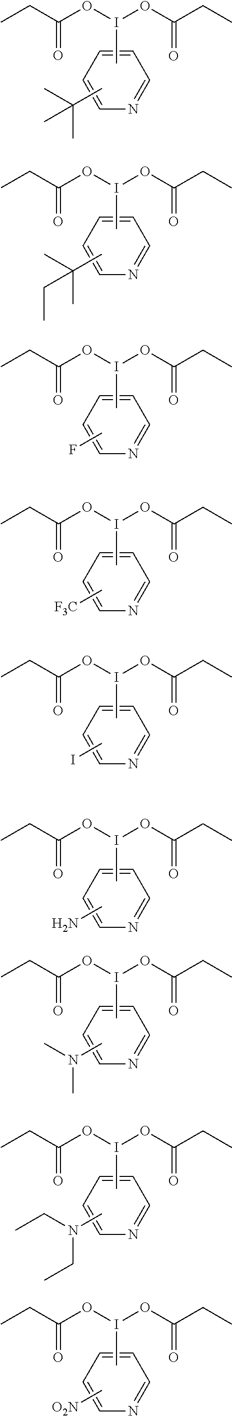

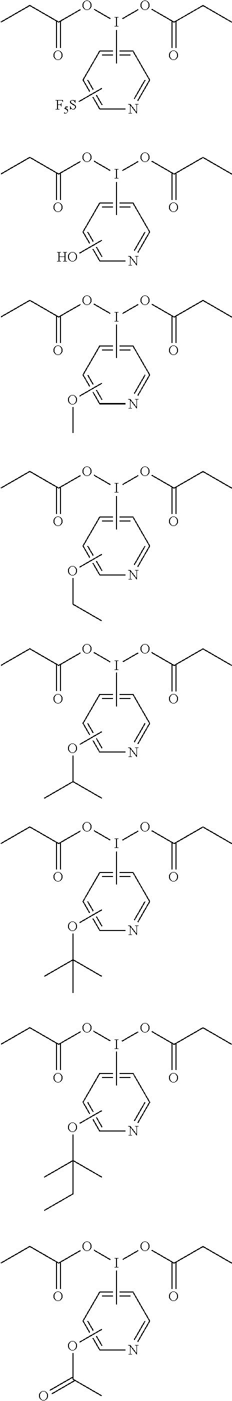

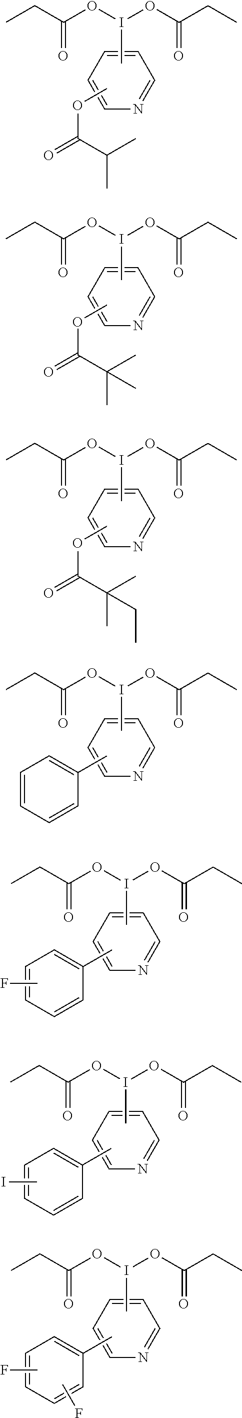

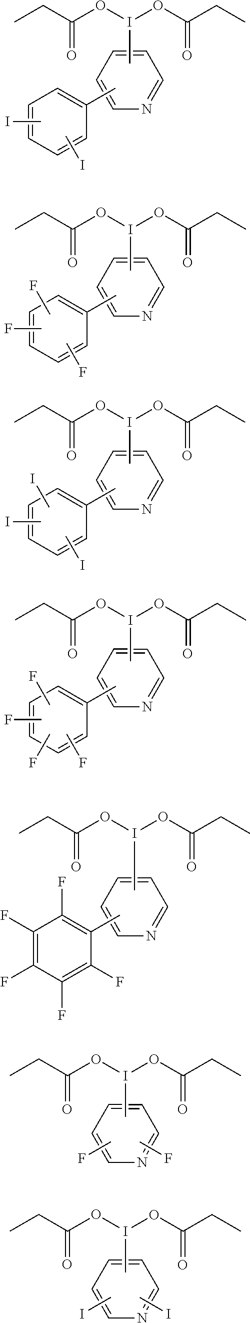

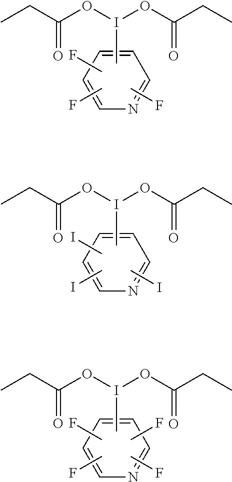

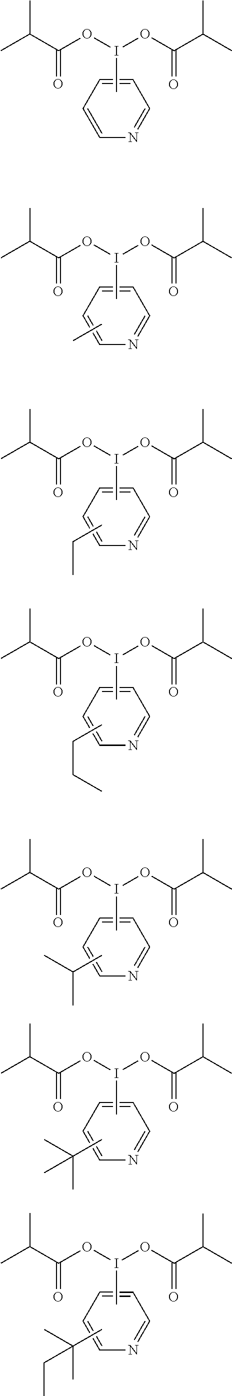

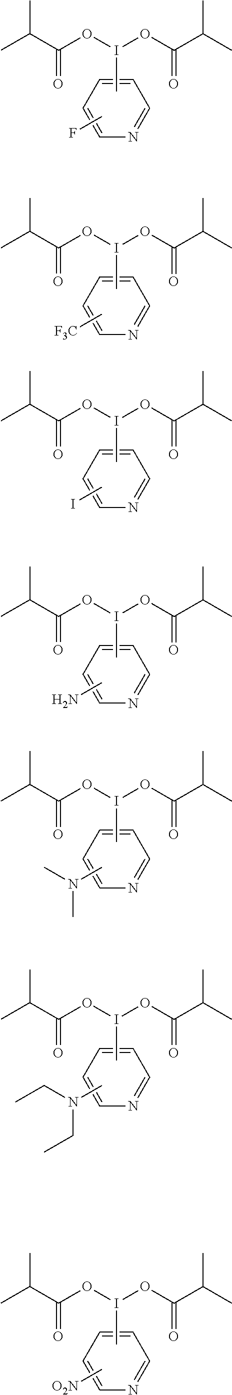

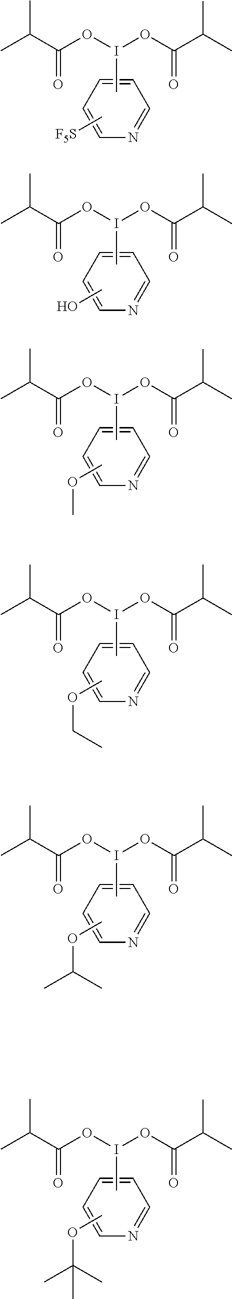

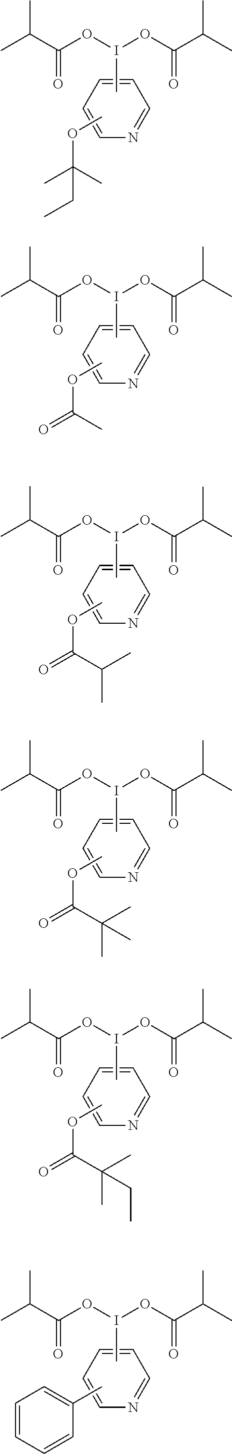

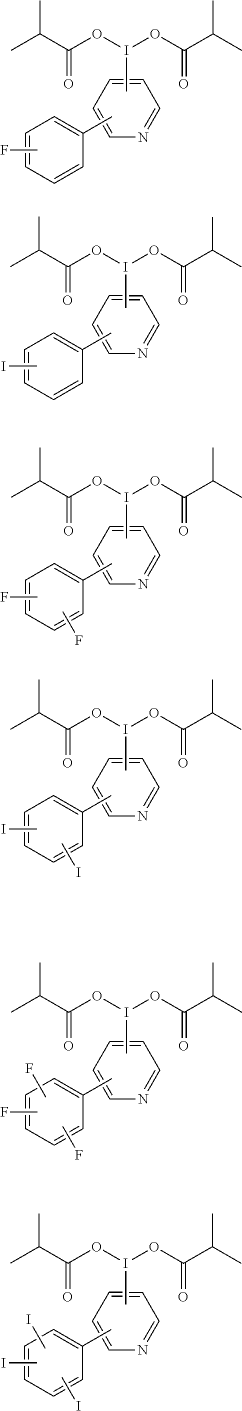

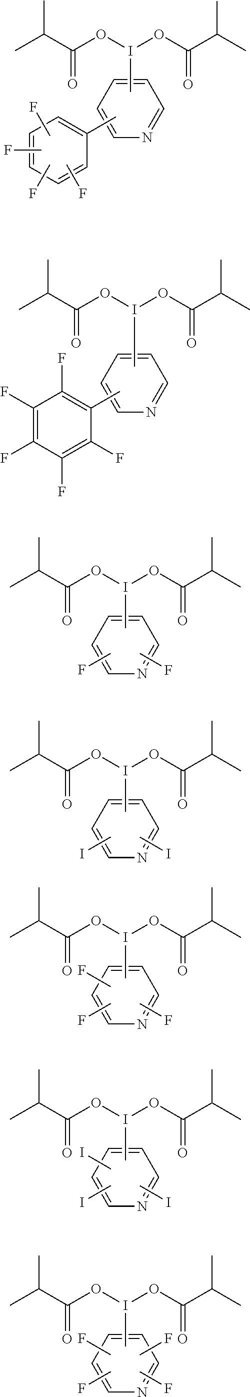

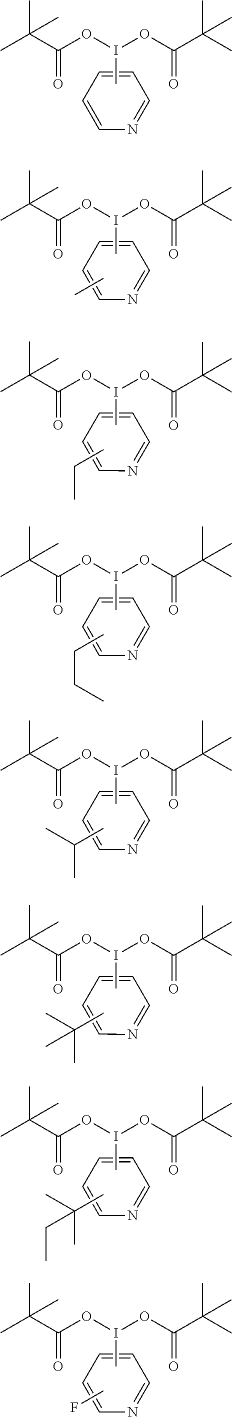

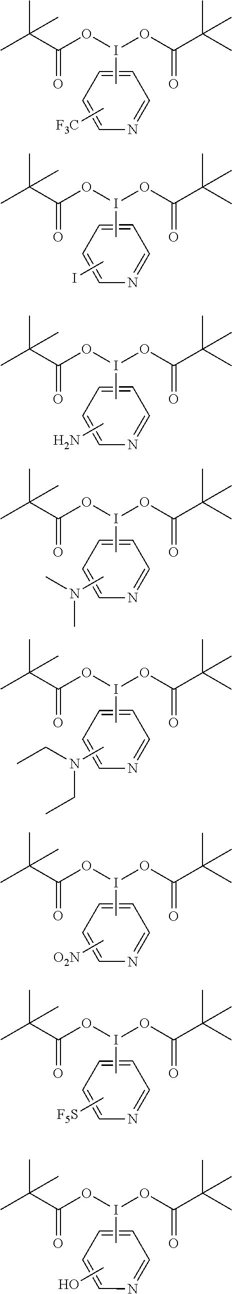

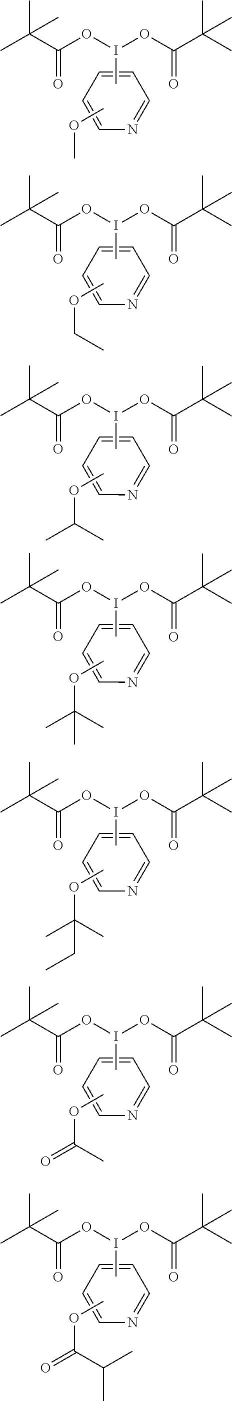

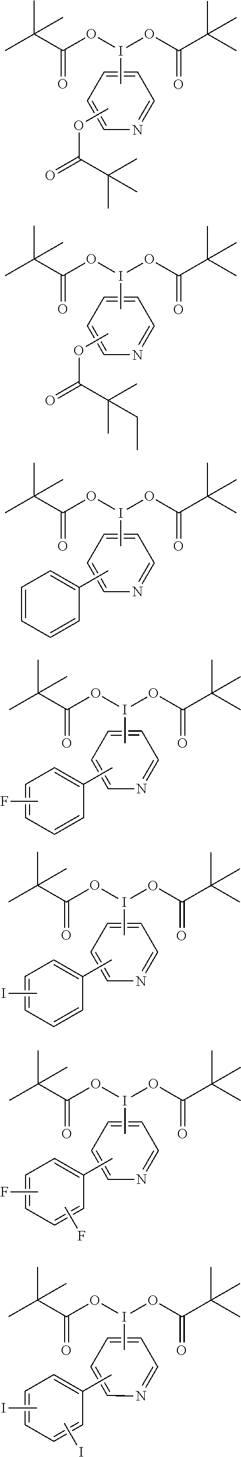

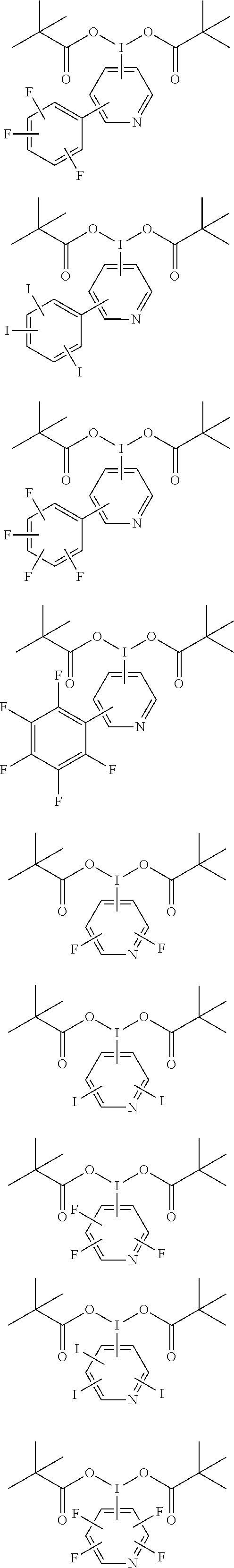

### [Method for Manufacturing Hypervalent Iodine Compound]

The above-described heterocycle-containing hypervalent iodine compounds are compounds in which a carboxylate is coordinated to hypervalent iodine, and can be synthesized according to a method for synthesizing a known aromatic hypervalent iodine compound such as (diacetoxyiodo)benzene. For example, synthesis of thiophene-type and pyrazole-type hypervalent iodine compounds can be carried out with reference to J. Org. Chem., 2000, 65, 8391-8394, and synthesis of pyridine-type hypervalent iodine compounds can be carried out with reference to U.S. Pat. No. 3,565,906A.

To give one example, a compound can be obtained by dispersing an iodinated heteroaromatic compound, such as 3-iodothiophene, in acetic acid, adding sodium perborate tetrahydrate thereto, stirring at 30 to 80° C. for 12 hours, and then appropriately performing operations such as solvent removal, extraction, and purification.

### [Carboxy-Group-Containing Compound]

The carboxy-group-containing compound is preferably a polymer including a repeating unit represented by the following formula (4) or a compound represented by the following formula (5).

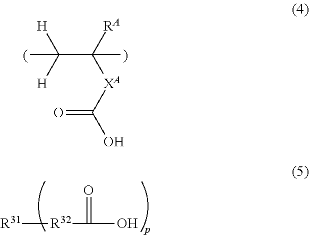

In the formulae, R^{A} represents a hydrogen atom, a halogen atom, a methyl group, or a trifluoromethyl group; X^{A} represents a single bond, a phenylene group, a naphthylene group, or *—C(═O)—O—X^{A1}—; X^{A1} represents a saturated hydrocarbylene group having 1 to 10 carbon atoms, a phenylene group, or a naphthylene group, the saturated hydrocarbylene group optionally containing a hydroxy group, an ether bond, an ester bond, or a lactone ring; “*” represents an attachment point to the carbon atom of the main chain; “p” represents 1, 2, 3, or 4; R³¹ represents a p-valent hydrocarbon group having 1 to 40 carbon atoms or a p-valent heterocyclic group having 2 to 40 carbon atoms, when “p” is 2, the R³¹ may be an ether bond, a carbonyl group, an azo group, a thioether bond, a carbonate bond, a carbamate bond, a sulfinyl group, or a sulfonyl group, where part or all of hydrogen atoms of the p-valent hydrocarbon group or the p-valent heterocyclic group may be substituted with a group containing a heteroatom, and part of —CH₂— of the p-valent hydrocarbon group may be substituted with a group containing a heteroatom; and R³² represents a single bond or a hydrocarbylene group having 1 to 20 carbon atoms, part or all of hydrogen atoms of the hydrocarbylene group may be substituted with a group containing a heteroatom, part of —CH₂— of the hydrocarbylene group may be substituted with a group containing a heteroatom, and when “p” is 2, 3, or 4, the R³²s are identical to or different from each other.

In the formula (4), R^{A} represents a hydrogen atom, a halogen atom, a methyl group, or a trifluoromethyl group; X^{A} represents a single bond, a phenylene group, a naphthylene group, or *—C(═O)—O—X^{A1}—; X^{A1} represents a saturated hydrocarbylene group having 1 to 10 carbon atoms, a phenylene group, or a naphthylene group, the saturated hydrocarbylene group optionally containing a hydroxy group, an ether bond, an ester bond, or a lactone ring; and “*” represents an attachment point to the carbon atom of the main chain.

In the formula (5), “p” represents 1, 2, 3, or 4.

In the formula (5), R³¹ represents a p-valent hydrocarbon group having 1 to 40 carbon atoms or a p-valent heterocyclic group having 2 to 40 carbon atoms, when “p” is 2, the R³¹ may be an ether bond, a carbonyl group, an azo group, a thioether bond, a carbonate bond, a carbamate bond, a sulfinyl group, or a sulfonyl group, where part or all of hydrogen atoms of the p-valent hydrocarbon group or the p-valent heterocyclic group may be substituted with a group containing a heteroatom, and part of —CH₂— of the p-valent hydrocarbon group may be substituted with a group containing a heteroatom.

In the formula (5), R³² represents a single bond or a hydrocarbylene group having 1 to 20 carbon atoms, part or all of hydrogen atoms of the hydrocarbylene group may be substituted with a group containing a heteroatom, part of —CH₂— of the hydrocarbylene group may be substituted with a group containing a heteroatom, and when “p” is 2, 3, or 4, the R³²s are identical to or different from each other.

The p-valent hydrocarbon group represented by R³¹ may be saturated or unsaturated, and may be linear, branched, or cyclic. The p-valent hydrocarbon group is a group obtained by “p” hydrogen atoms being removed from a hydrocarbon. Examples of the hydrocarbon include alkanes having 1 to 40 carbon atoms, alkenes having 2 to 40 carbon atoms, alkynes having 2 to 40 carbon atoms, cyclic saturated hydrocarbons having 3 to 40 carbon atoms, cyclic unsaturated hydrocarbons having 3 to 40 carbon atoms, and aromatic hydrocarbons having 6 to 40 carbon atoms.

Examples of the alkanes having 1 to 40 carbon atoms include methane, ethane, propane, butane, pentane, hexane, heptane, octane, nonane, decane, and structural isomers thereof.

Examples of the alkenes having 2 to 40 carbon atoms include ethylene, propylene, butene, pentene, hexene, heptene, octene, nonene, decene, and structural isomers thereof.

Examples of the alkynes having 2 to 40 carbon atoms include acetylene, propyne, butyne, pentyne, hexyne, heptyne, octyne, nonyne, decyne, and structural isomers thereof.

Examples of the cyclic saturated hydrocarbons having 3 to 40 carbon atoms include cyclopropane, cyclobutane, cyclohexane, cycloheptane, cyclooctane, adamantane, and norbornane.

Examples of the cyclic unsaturated hydrocarbons having 3 to 40 carbon atoms include cyclopropene, cyclobutene, cyclopentene, cyclohexene, cycloheptene, cyclooctene, and norbornene.

Examples of the aromatic hydrocarbons having 6 to 40 carbon atoms include benzene, naphthalene, and biphenyl.

The p-valent heterocyclic group represented by R³¹ is a group obtained by removing “p” hydrogen atom from a heterocyclic compound. Examples of the heterocyclic compound include furan, pyridine, pyrazole, and thiazolidine.

Part or all of the hydrogen atoms of the p-valent hydrocarbon group or the p-valent heterocyclic group may be substituted with a group containing a heteroatom such as an oxygen atom, a sulfur atom, a nitrogen atom, or a halogen atom. The resulting p-valent hydrocarbon group or p-valent heterocyclic group may contain a hydroxy group, a cyano group, a fluorine atom, a chlorine atom, a bromine atom, an iodine atom, etc. Furthermore, part of the —CH₂-constituting the p-valent hydrocarbon group may be substituted with a group containing a heteroatom such as an oxygen atom, a sulfur atom, or a nitrogen atom. The resulting p-valent hydrocarbon group may contain a carbonyl group, an ether bond, a thioether bond, an ester bond, a sulfonic acid ester bond, a carbonate bond, a carbamate bond, a lactone ring, a sultone ring, a carboxylic acid anhydride (—C(═O)—O—C(═O)—), etc.

The hydrocarbylene group represented by R³² may be saturated or unsaturated, and may be linear, branched, or cyclic. Specific examples thereof include: alkanediyl groups having 1 to 20 carbon atoms, such as a methanediyl group, an ethane-1,1-diyl group, an ethane-1,2-diyl group, a propane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, and a dodecane-1,12-diyl group; cyclic saturated hydrocarbylene groups having 3 to 20 carbon atoms, such as a cyclopentanediyl group, a cyclohexanediyl group, a norbornanediyl group, and an adamantanediyl group; unsaturated aliphatic hydrocarbylene groups having 2 to 20 carbon atoms, such as a vinylene group and a propene-1,3-diyl group; arylene groups having 6 to 20 carbon atoms, such as a phenylene group and a naphthylene group; and groups obtained by combining these groups. Furthermore, part or all of the hydrogen atoms of the hydrocarbylene group may be substituted with a group containing a heteroatom, such as an oxygen atom, a sulfur atom, a nitrogen atom, or a halogen atom, and part of the —CH₂— constituting the hydrocarbylene group may be substituted with a group containing a heteroatom such as an oxygen atom, a sulfur atom, or a nitrogen atom. The resulting hydrocarbylene group may contain a hydroxy group, a cyano group, a fluorine atom, a chlorine atom, a bromine atom, an iodine atom, a carbonyl group, an ether bond, a thioether bond, an ester bond, a sulfonic acid ester bond, a carbonate bond, a carbamate bond, a lactone ring, a sultone ring, a carboxylic anhydride, etc.

Among carboxylic acid compounds represented by the formula (5), compounds in which “p” is 2, 3, or 4 are preferable. In this case, a strong resist film having a high molecular weight can be easily formed when the carboxylic acid compound is mixed with the hypervalent iodine compound, and therefore, such compounds are preferable from the viewpoints of etching resistance and developer resistance.

Specific examples of the carboxy-group-containing repeating unit represented by the formula (4) include the following, but are not limited thereto. Note that, in the following formulae, R^{A} is as defined above.

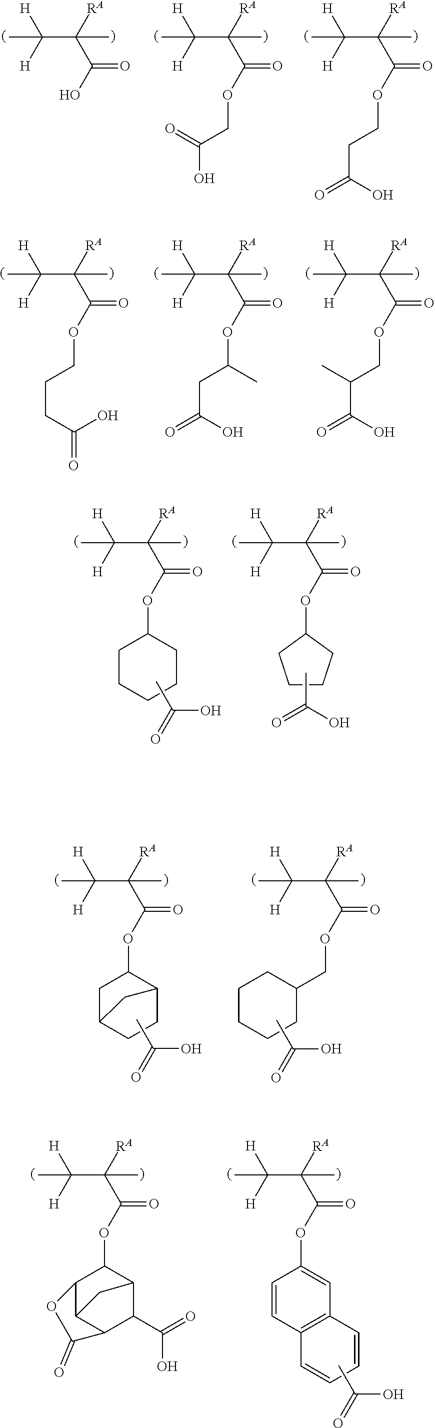

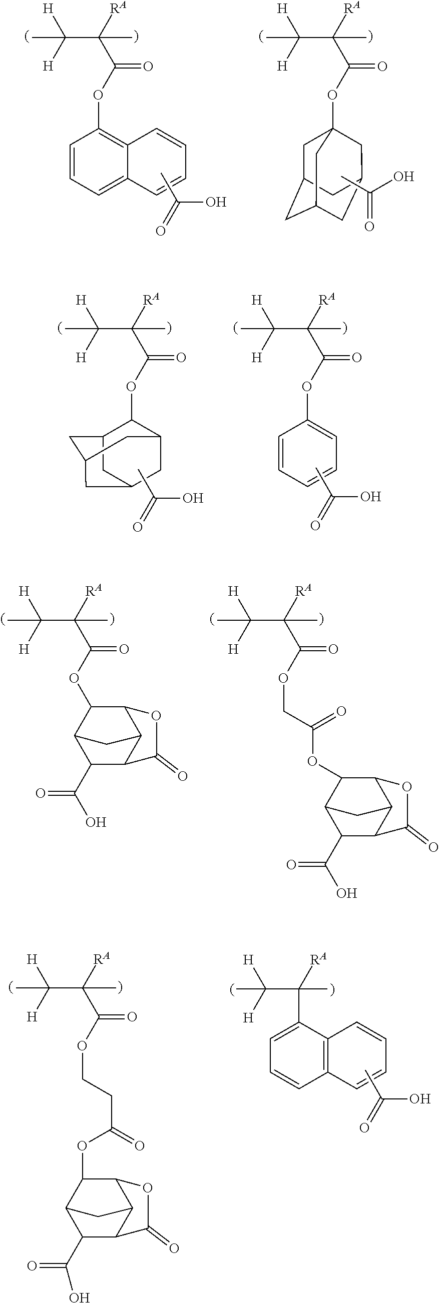

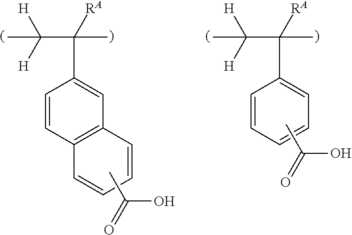

Examples of the carboxylic acid compound represented by the formula (5) include the following, but are not limited thereto. The carboxylic acid compound may be a commercially available product or a synthesized compound.

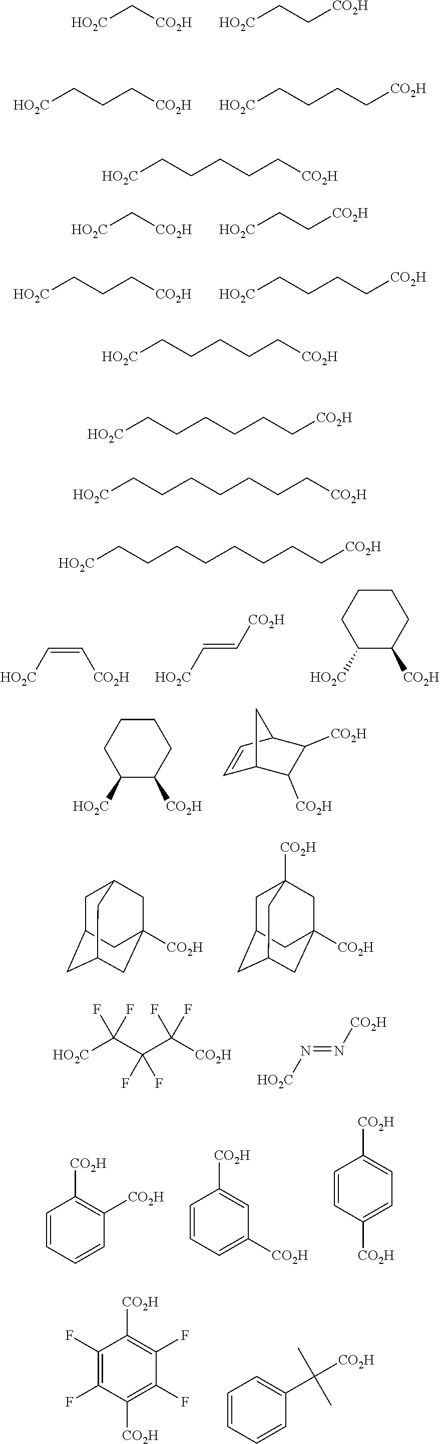

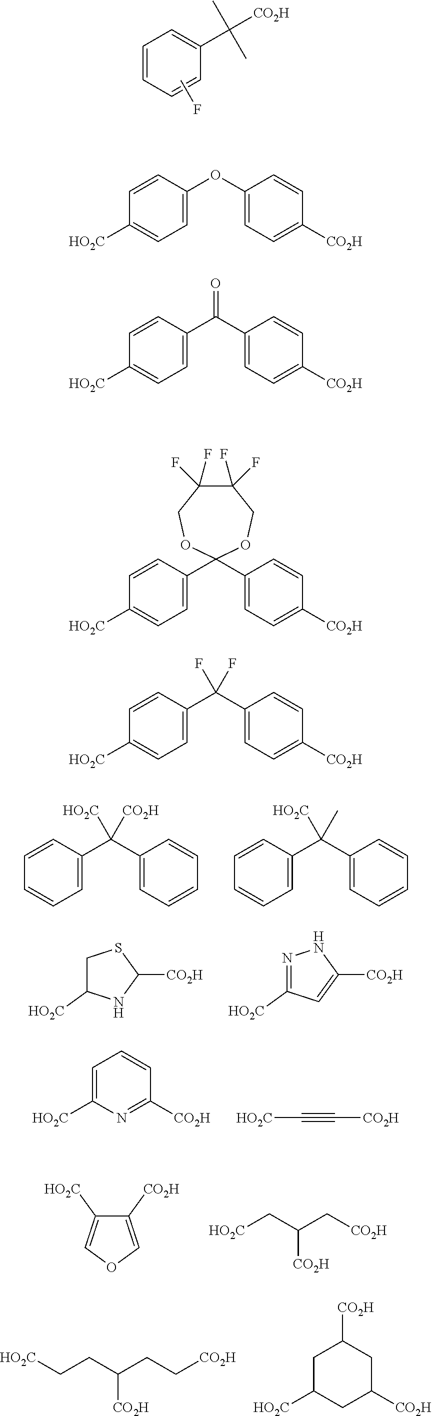

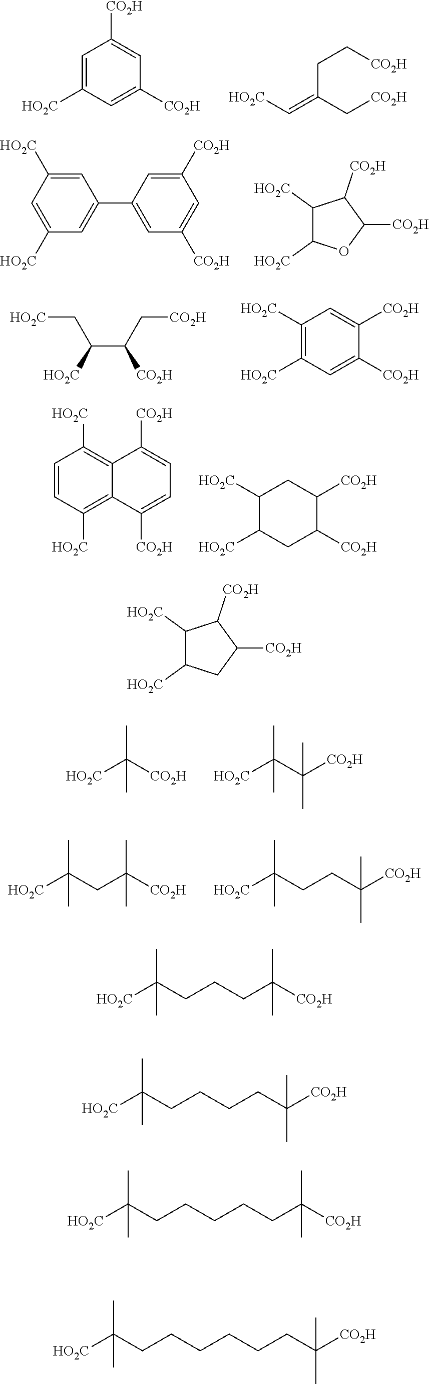

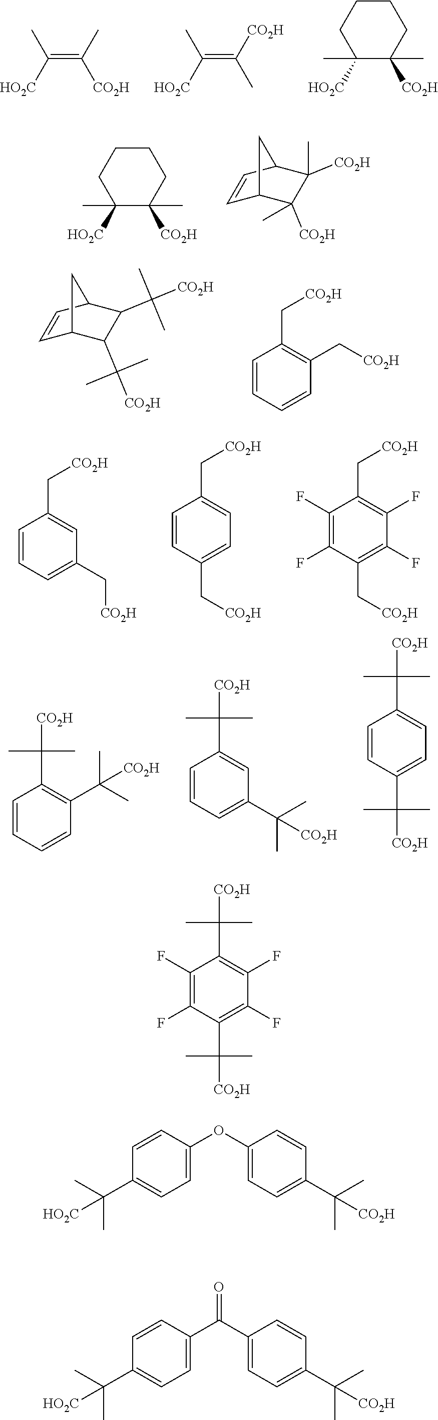

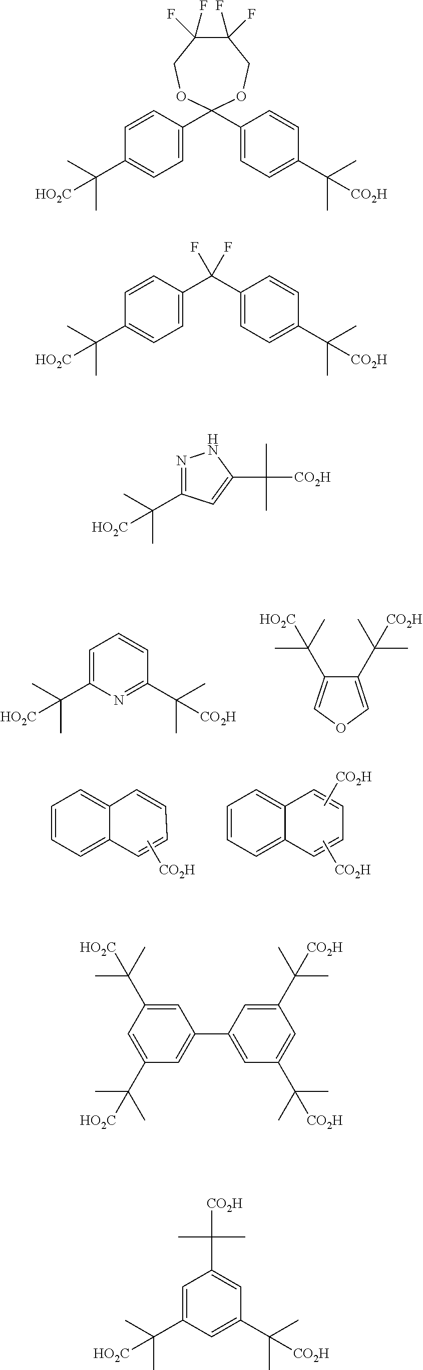

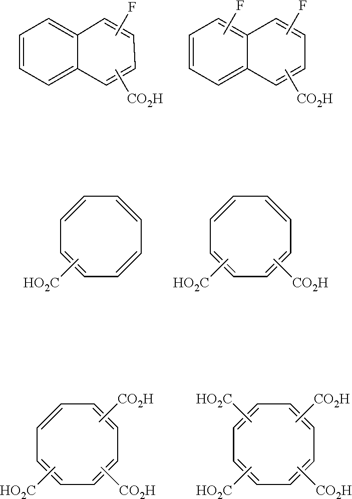

The carboxy-group-containing polymer including the repeating unit represented by the formula (4) may further include other repeating units (hereinafter, also referred to as other repeating units). The other repeating units are not particularly limited, but those which may enhance the solubility of the polymer in a solvent are preferable, because of the polymer being hardly soluble when having only a repeating unit having a carboxy group. As the other repeating units, repeating units having a cyclic structure and repeating units including a styrene skeleton, the units having a rigid skeleton being expected to have high etching resistance, are preferable.

Specific examples of the other repeating units include the following, but are not limited thereto. Note that, in the following formulae, R^{A} is as defined above and each X^{B} independently represents —CH₂— or —O—.

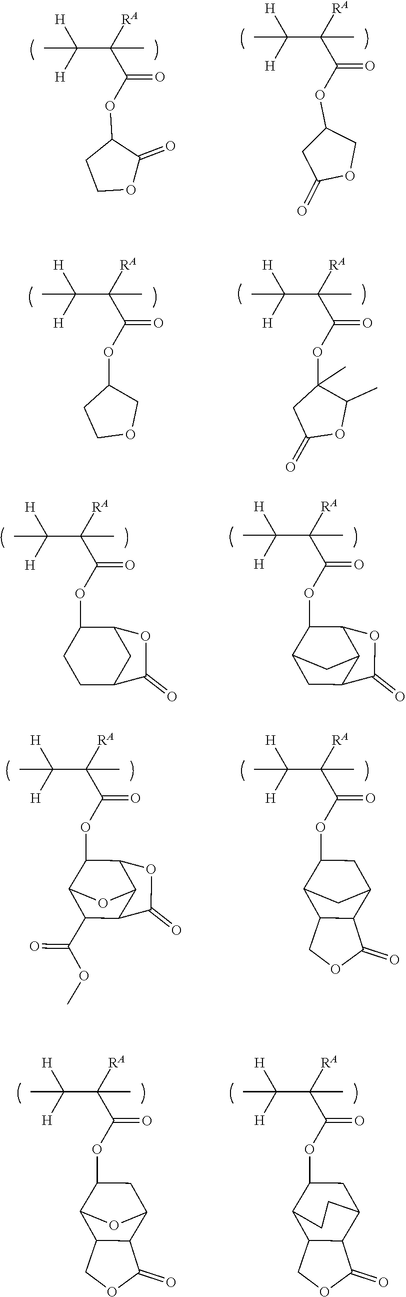

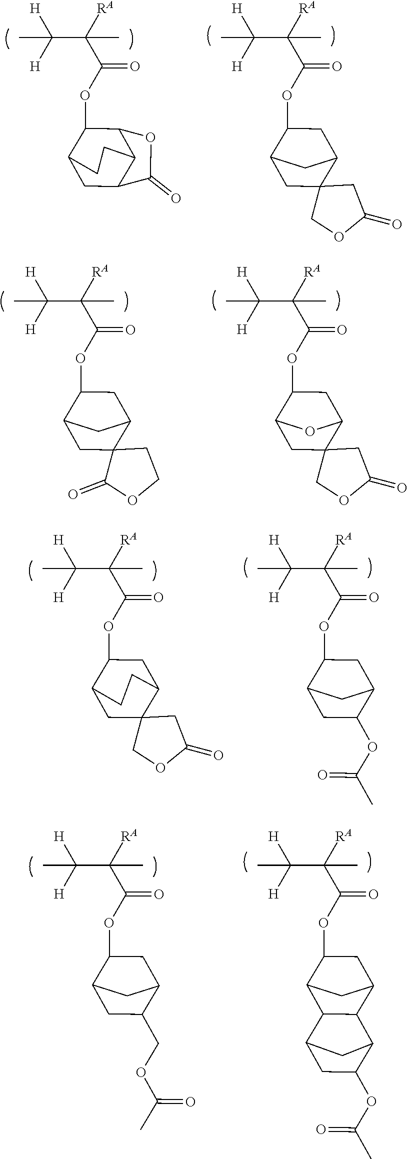

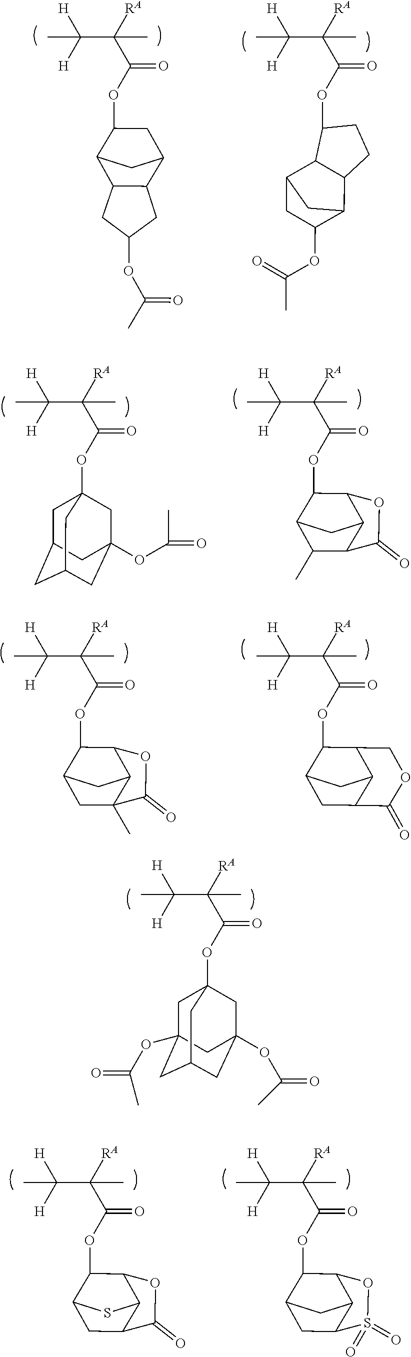

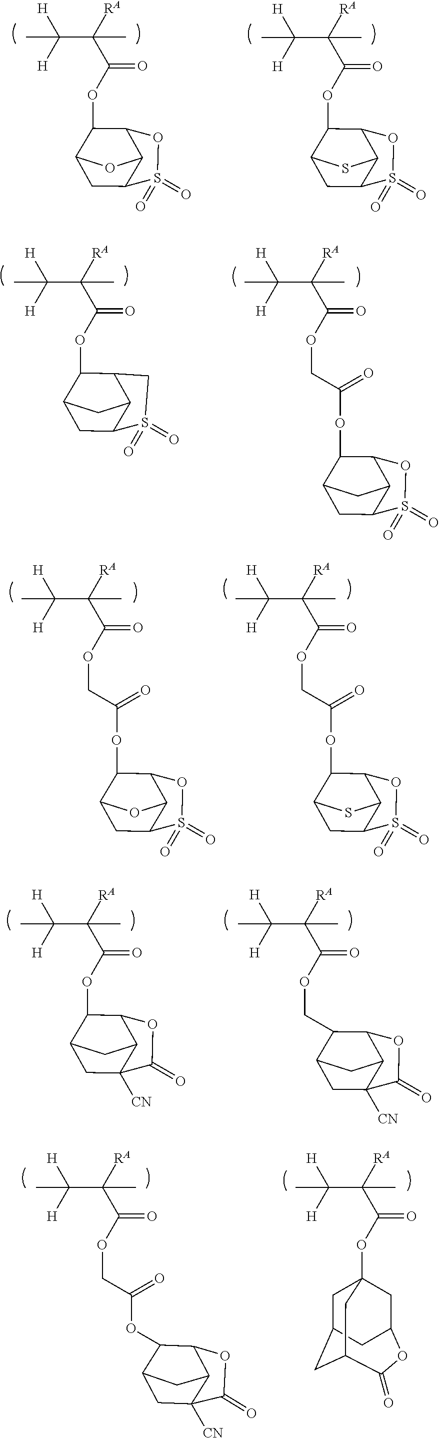

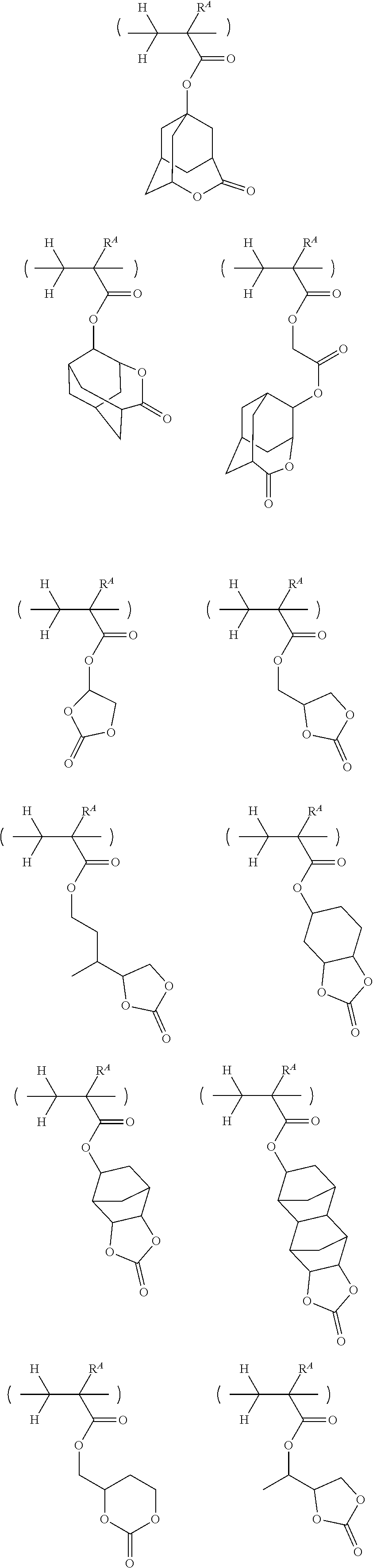

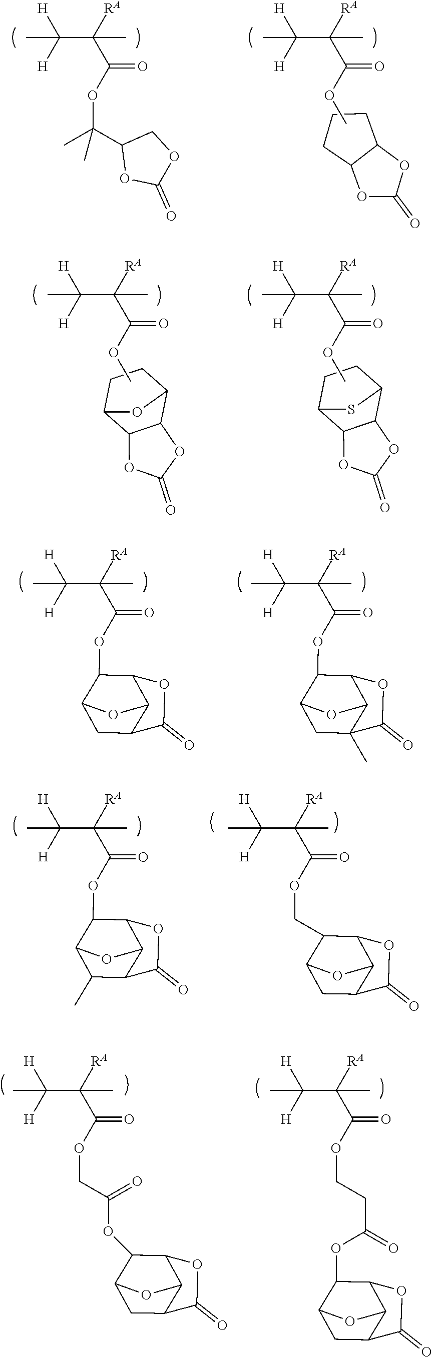

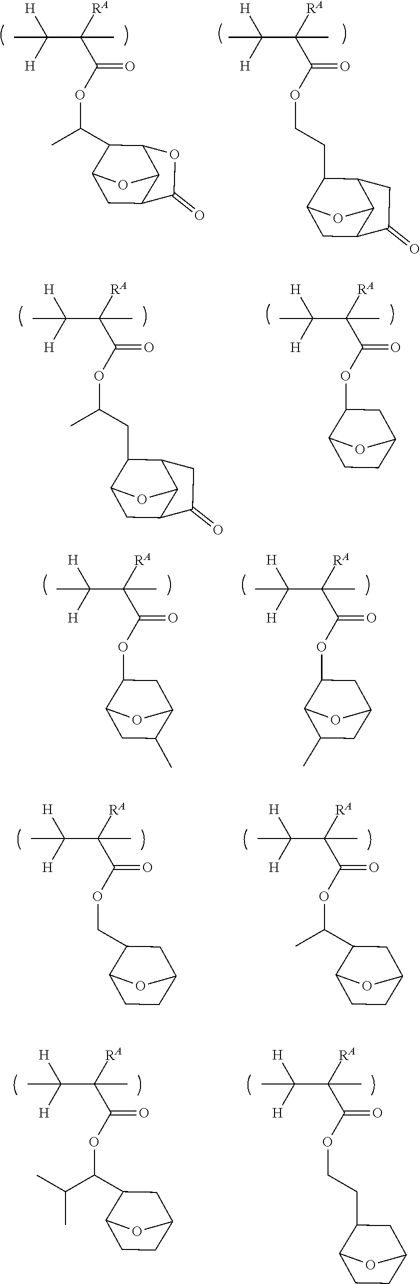

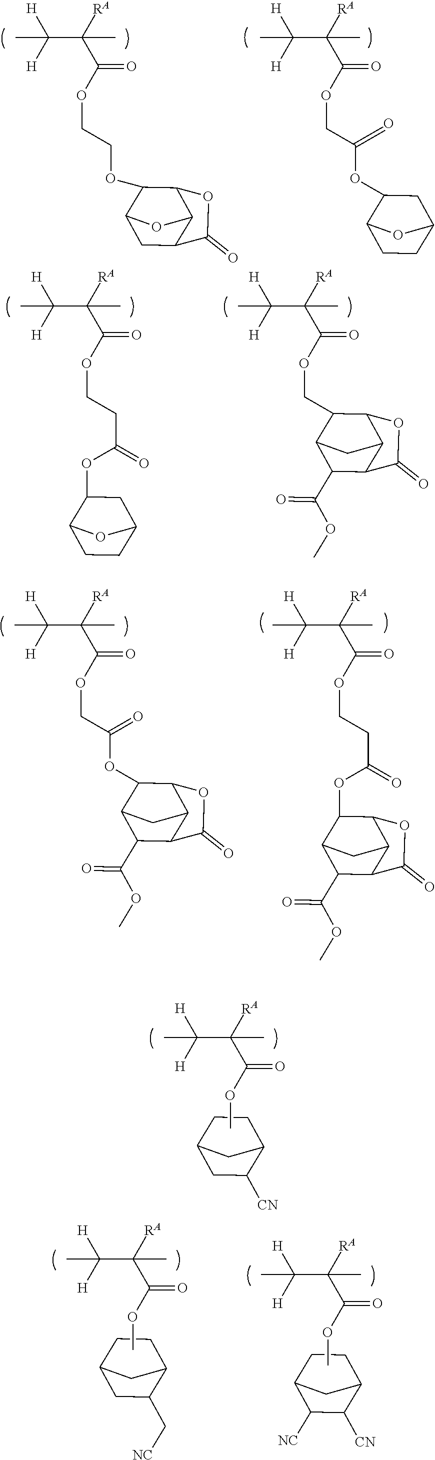

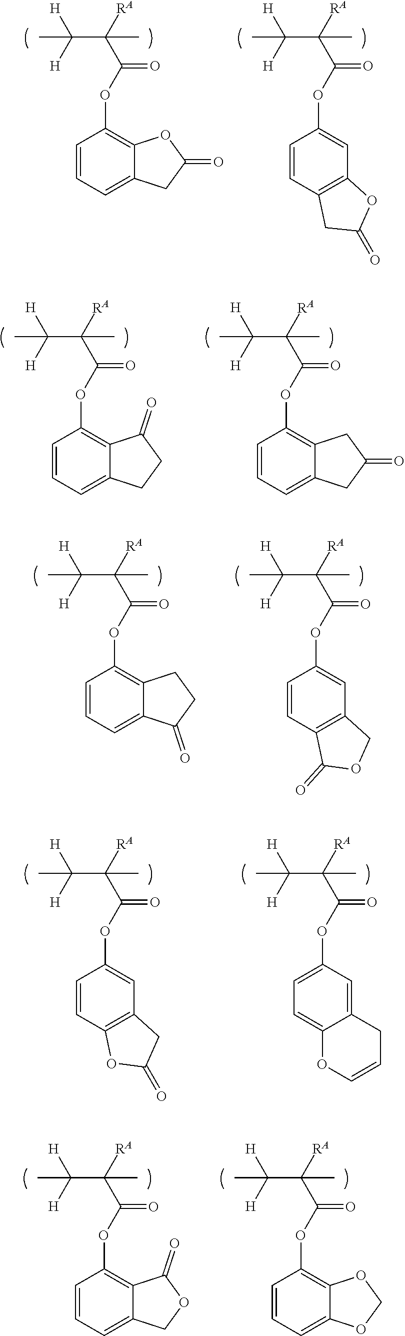

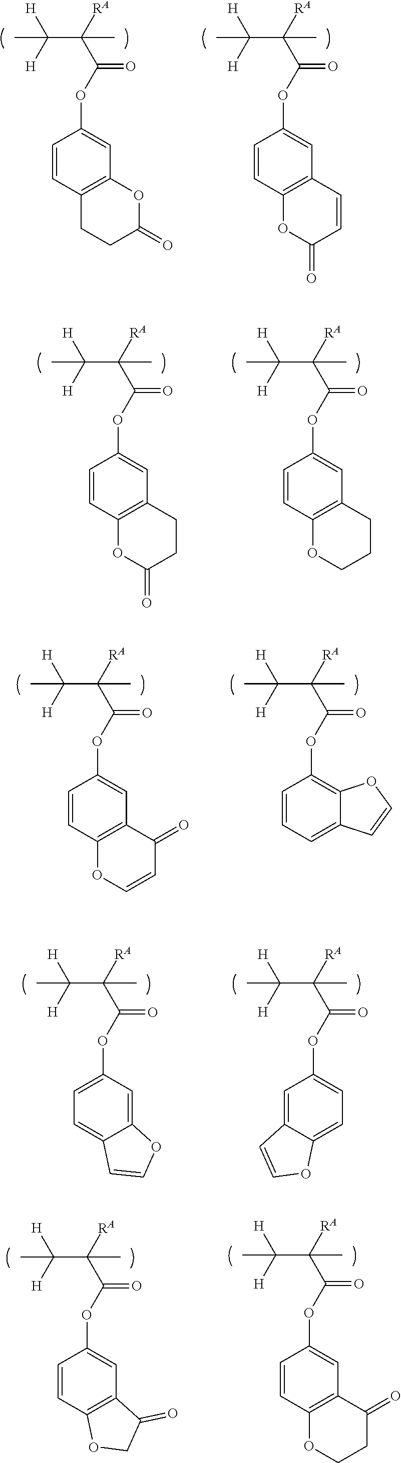

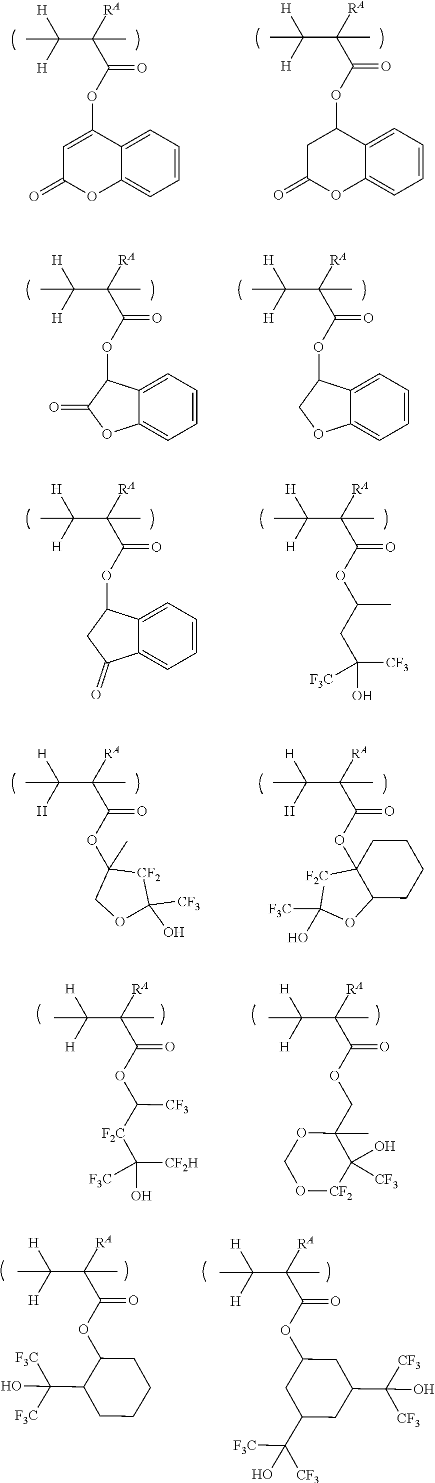

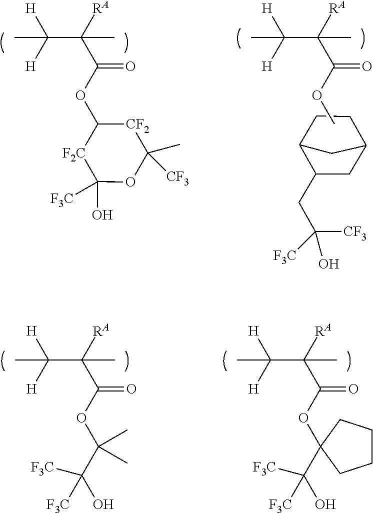

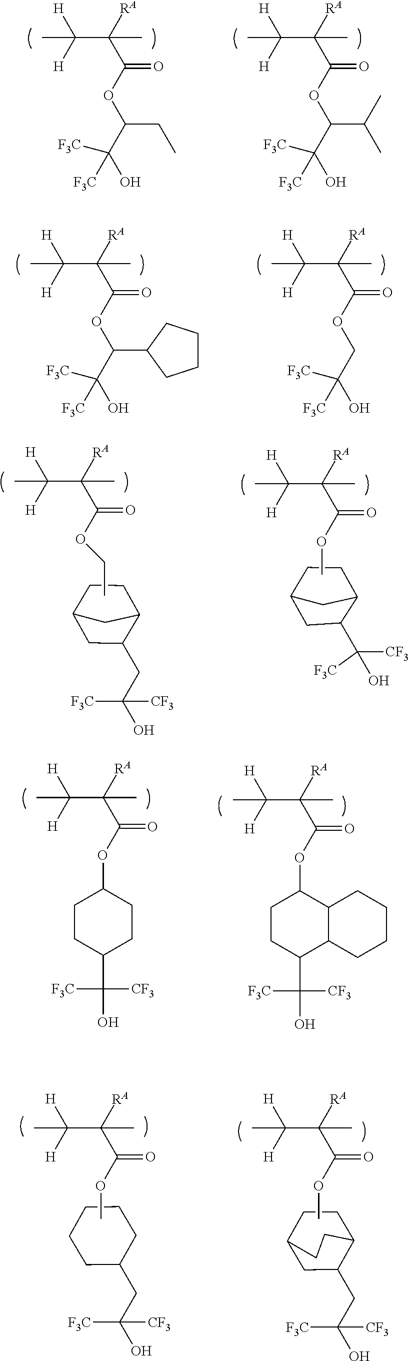

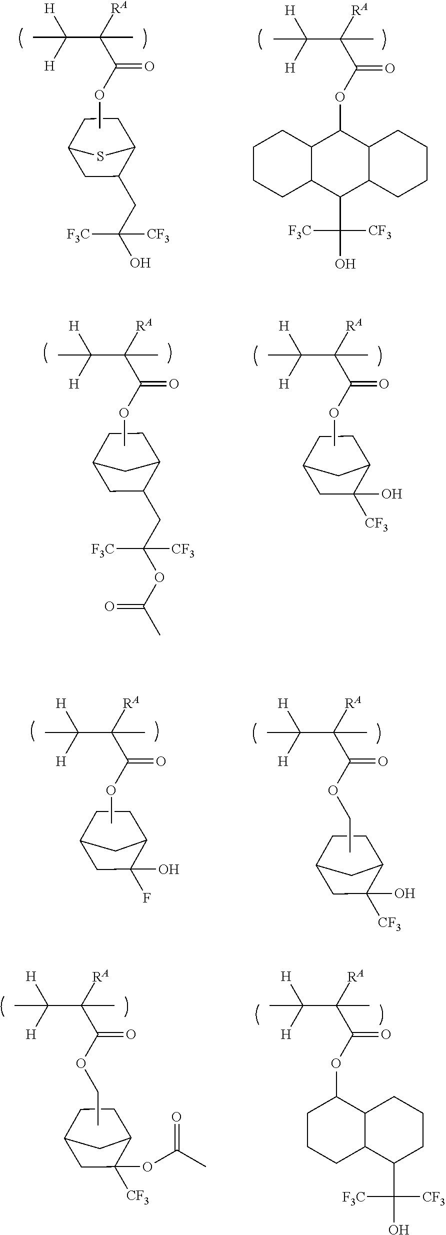

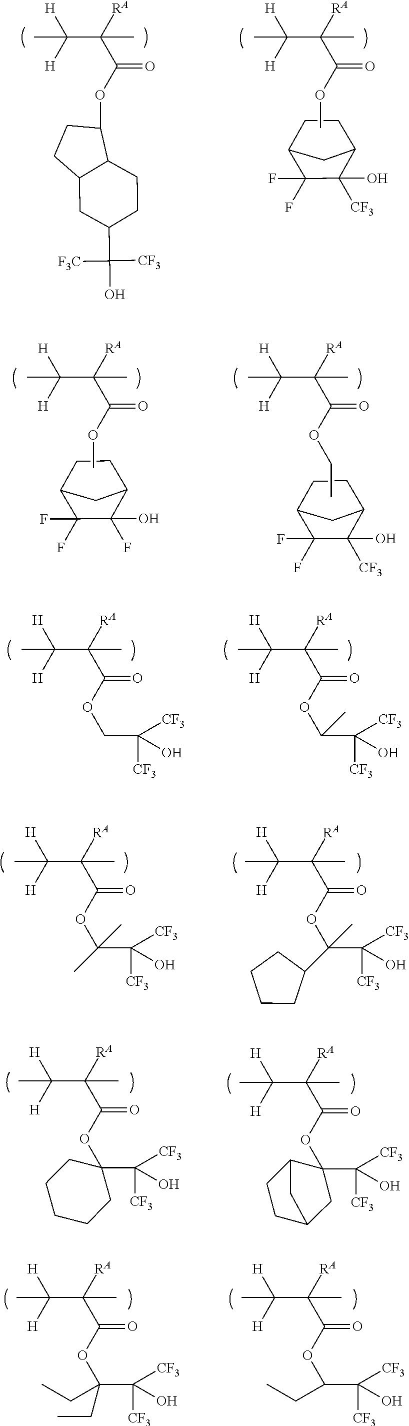

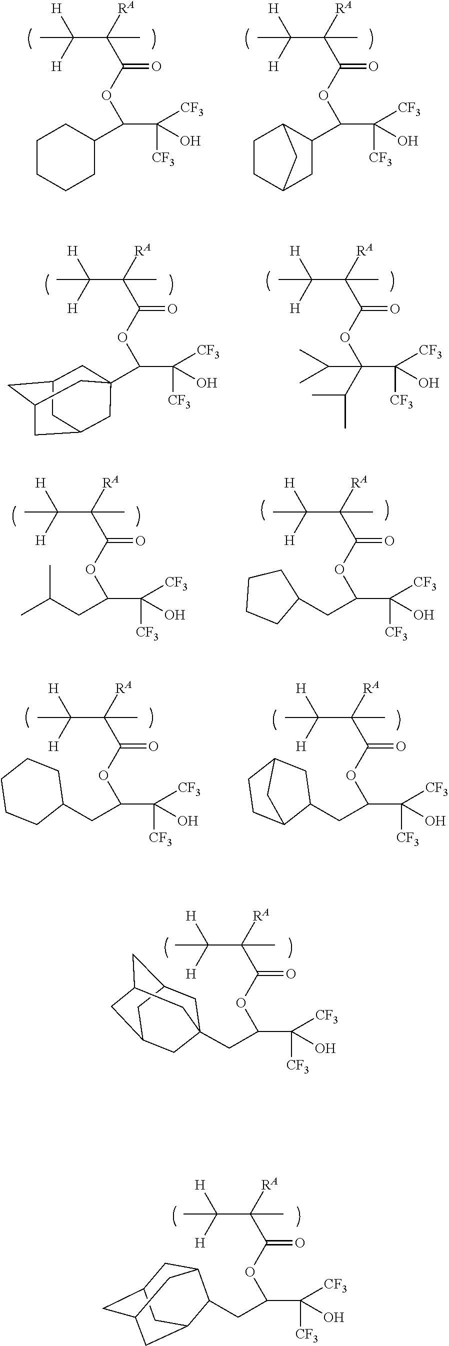

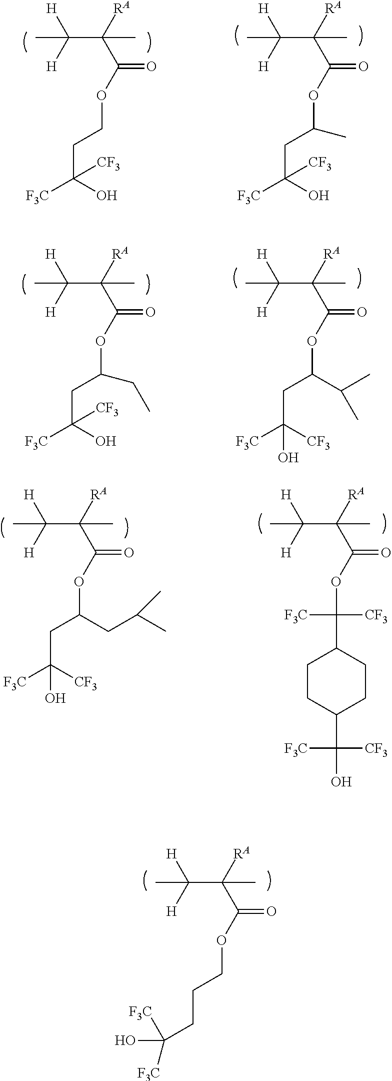

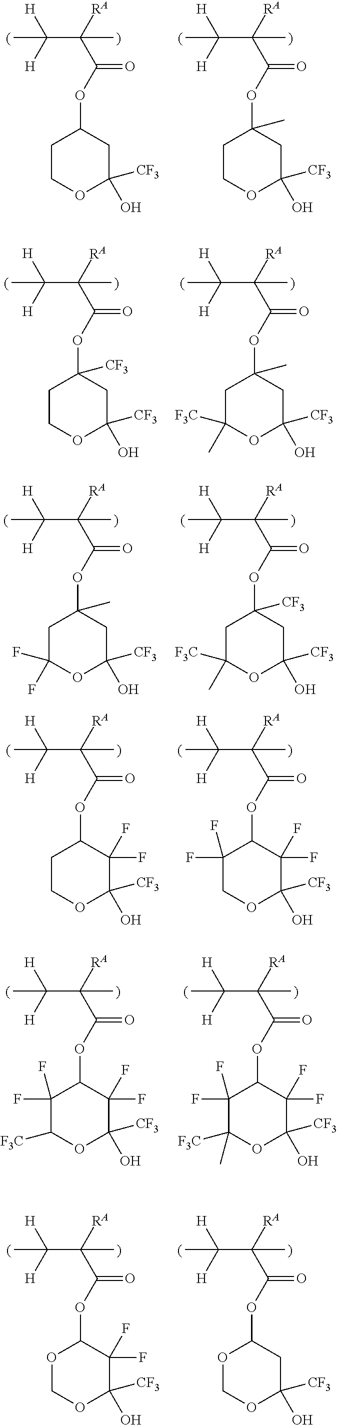

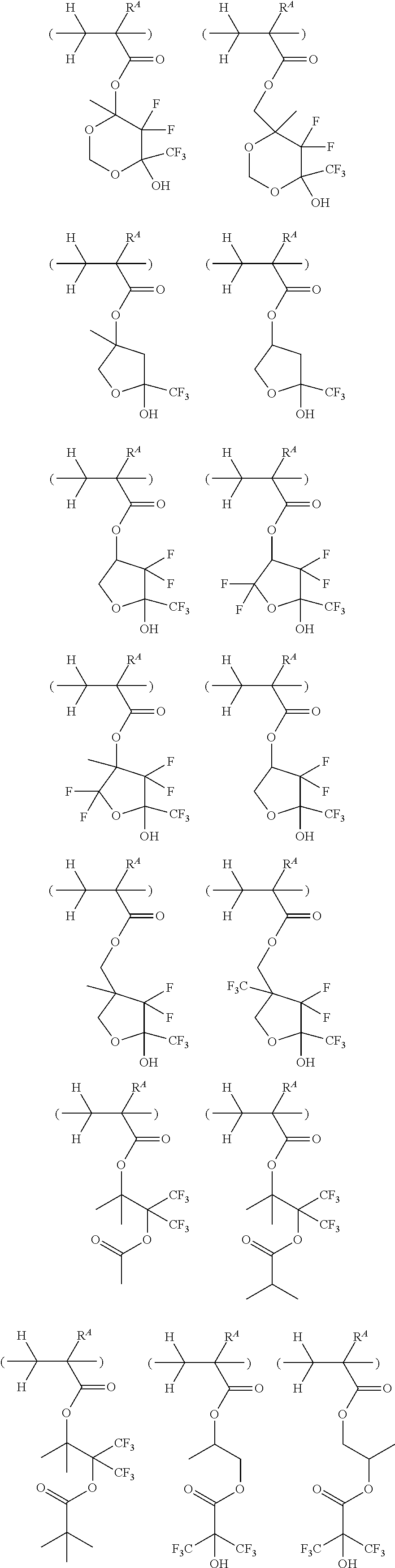

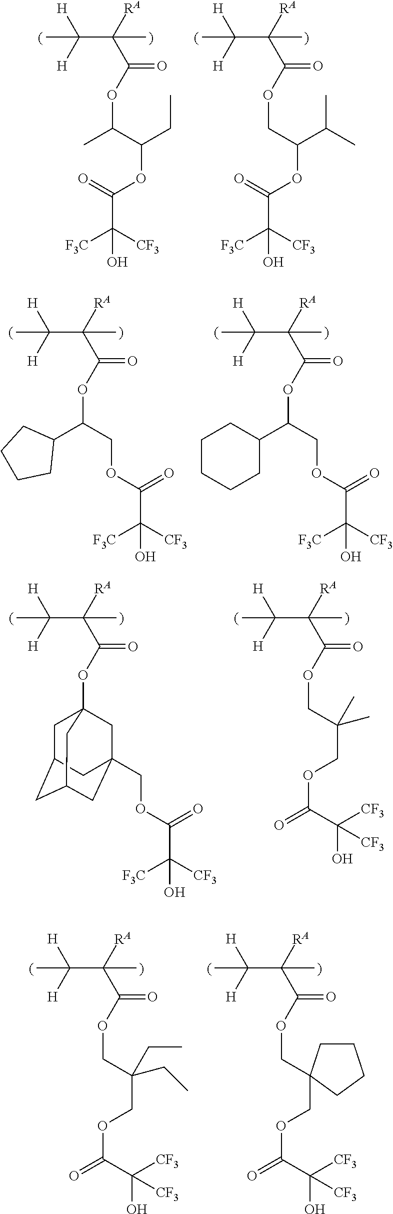

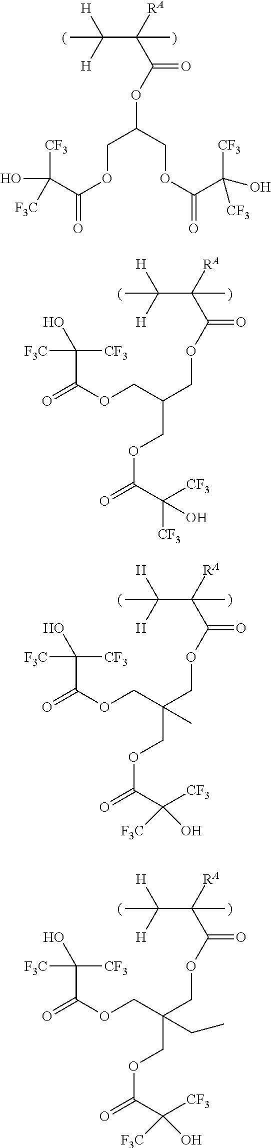

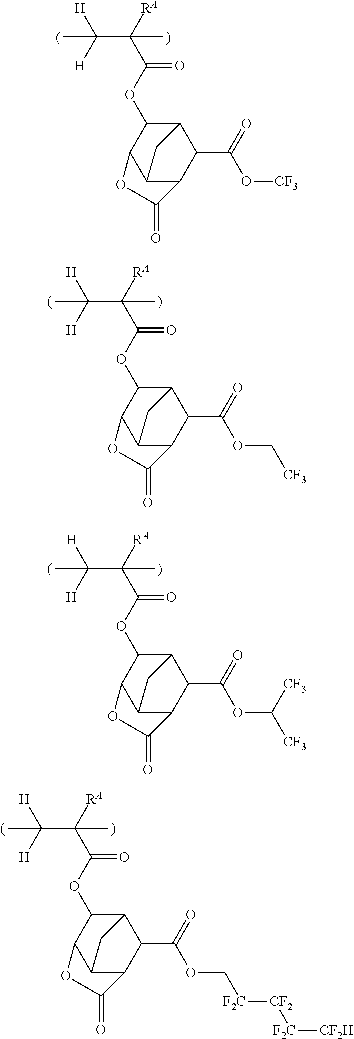

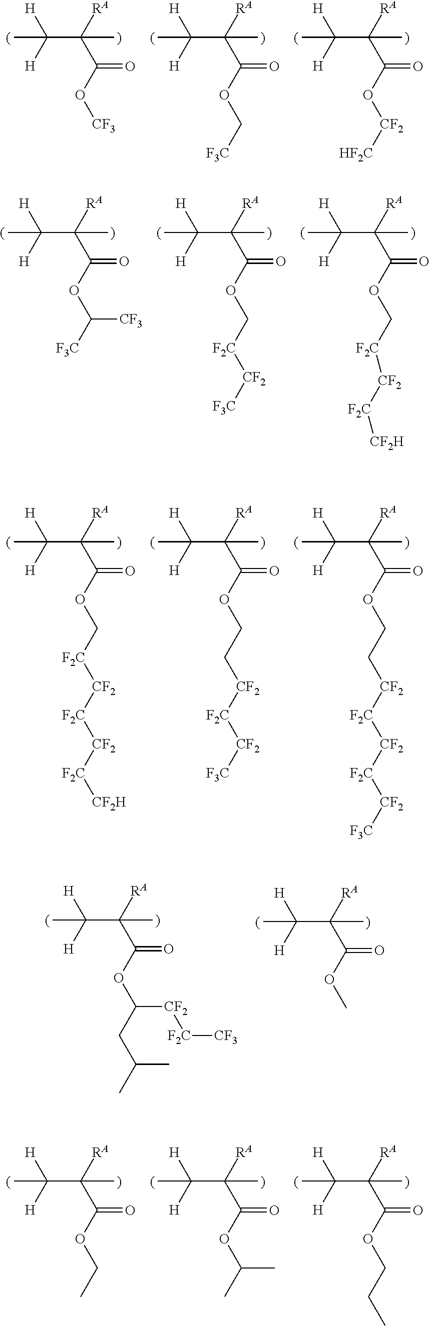

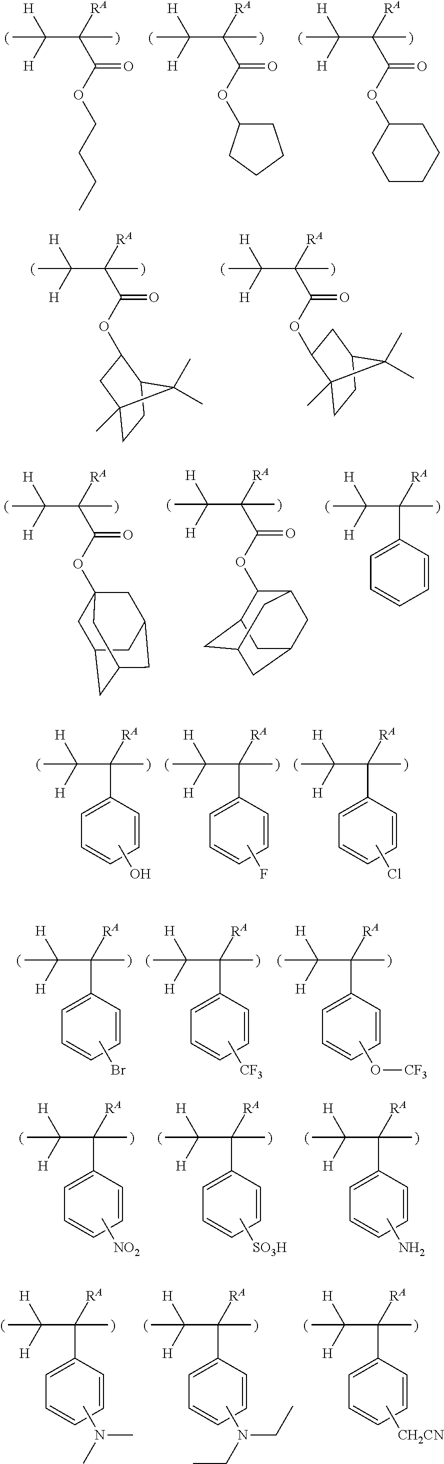

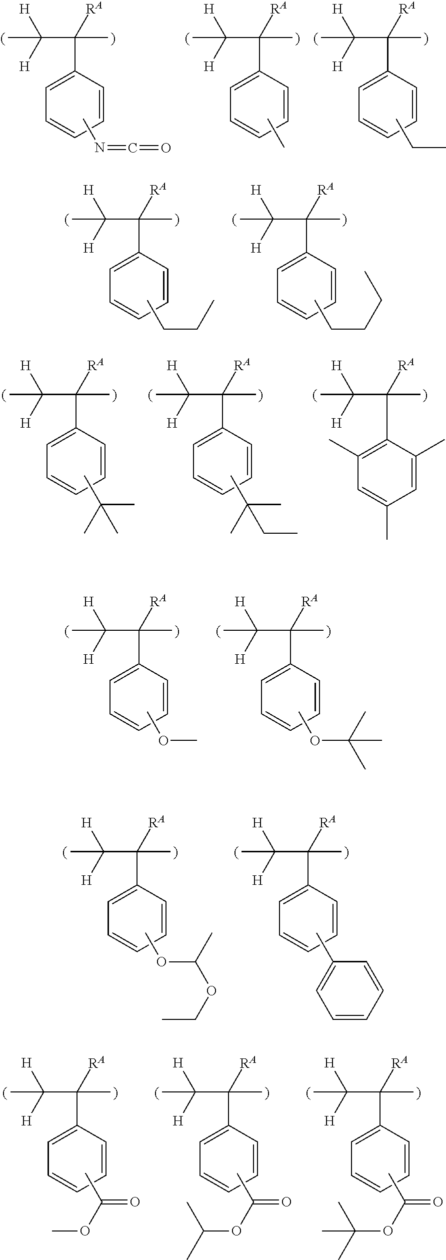

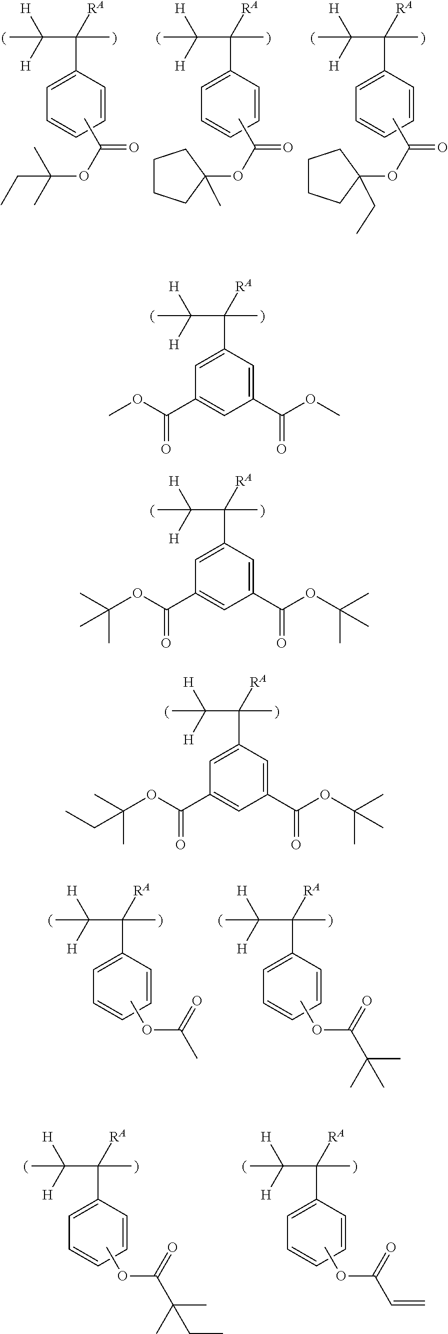

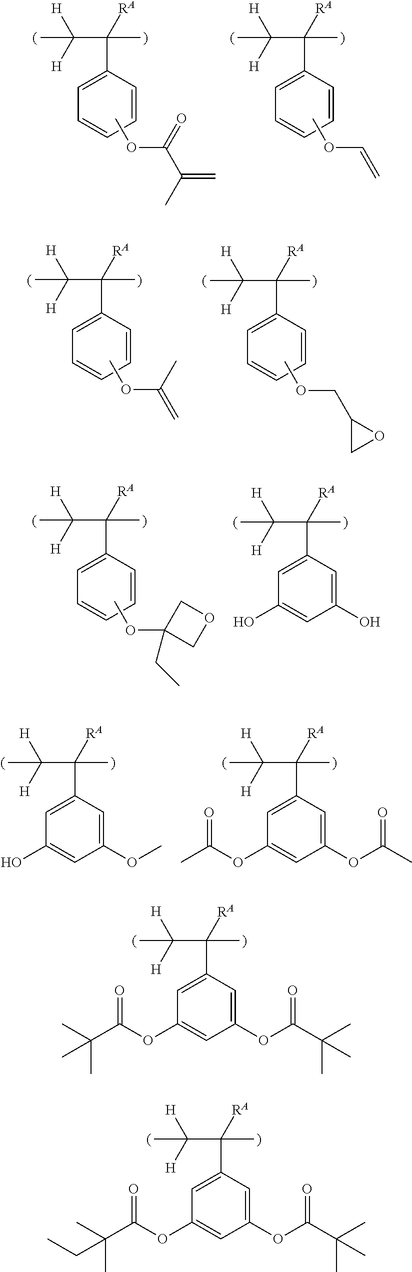

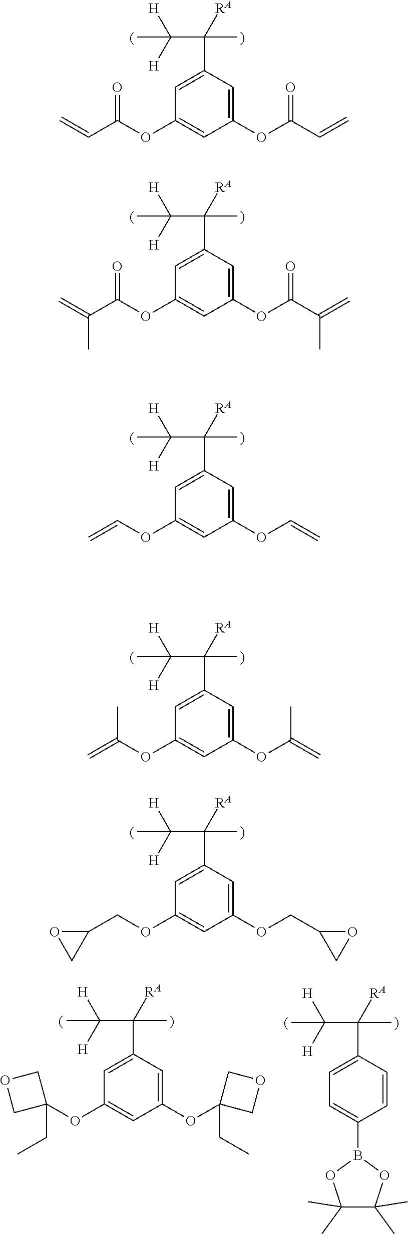

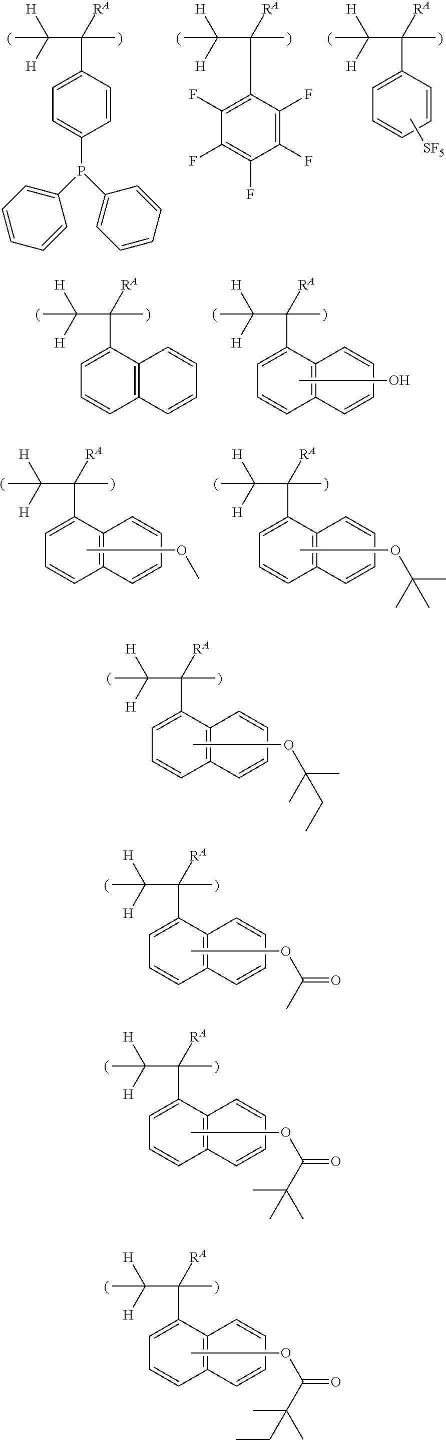

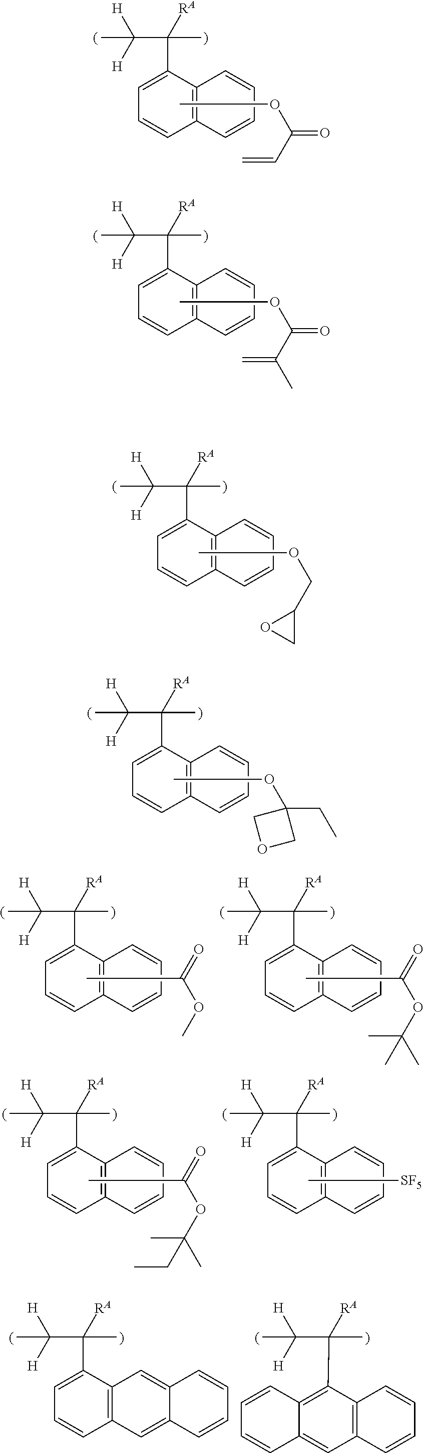

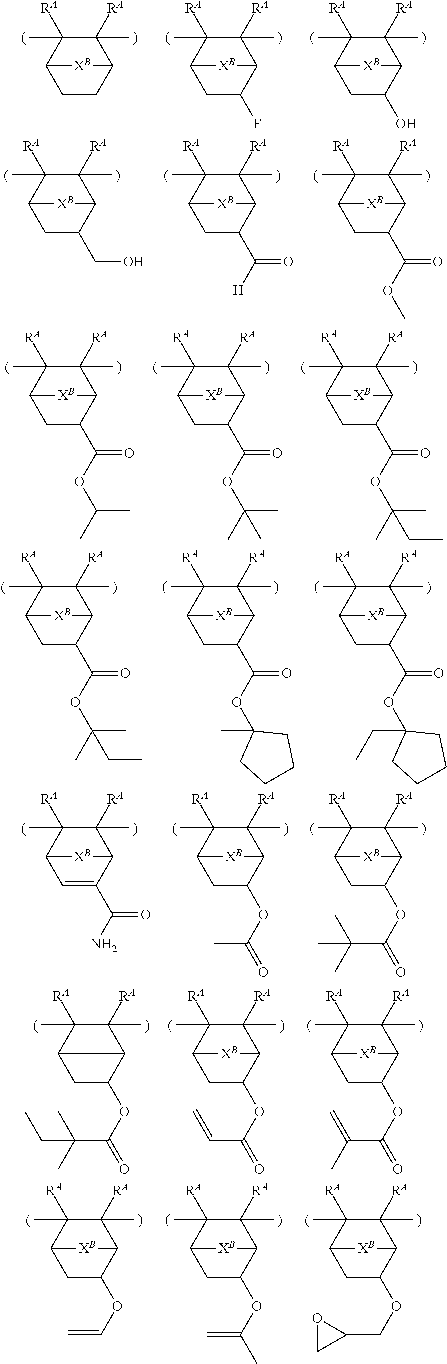

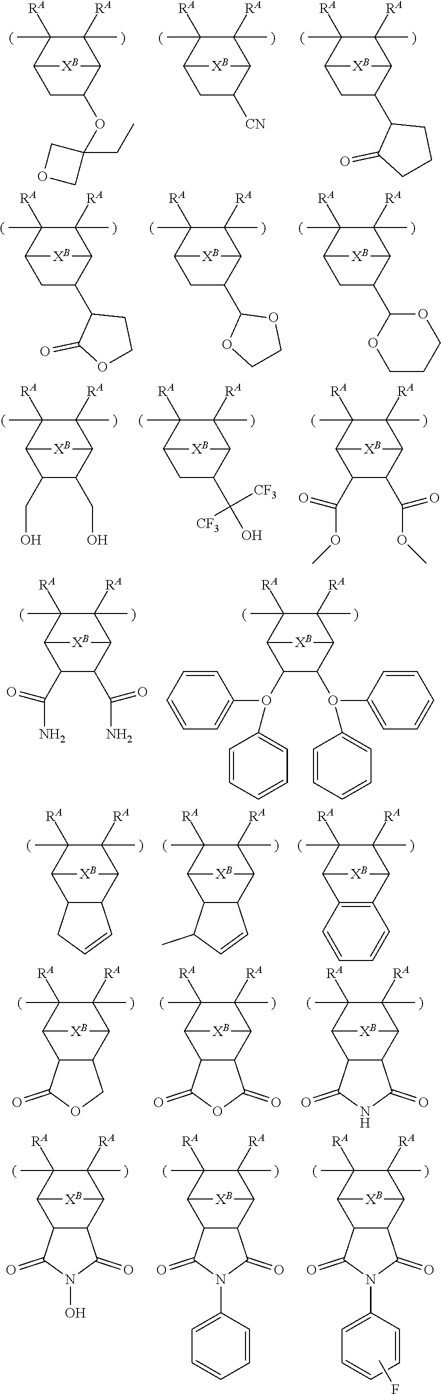

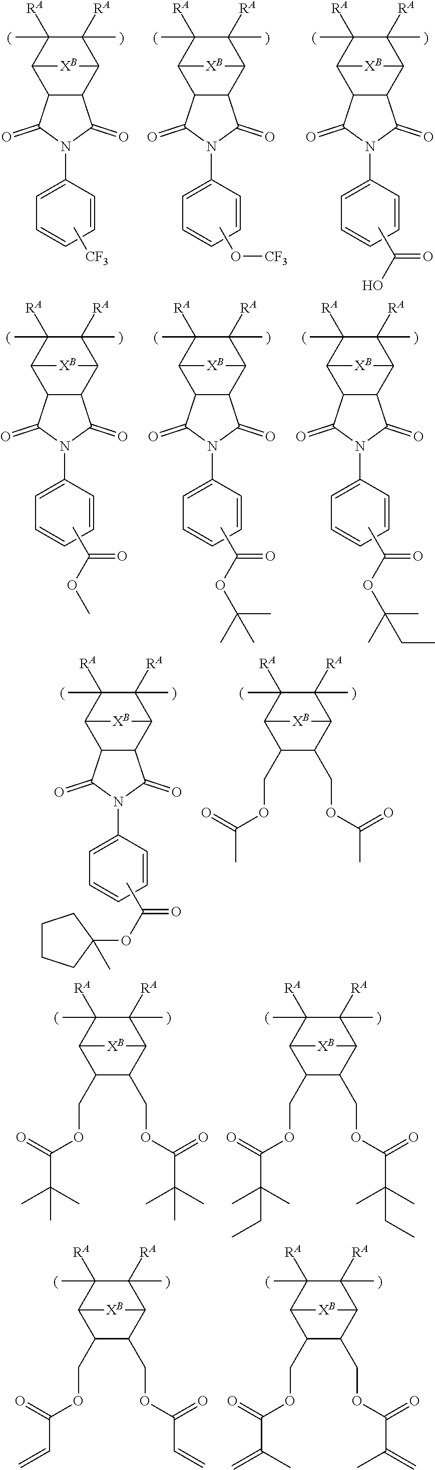

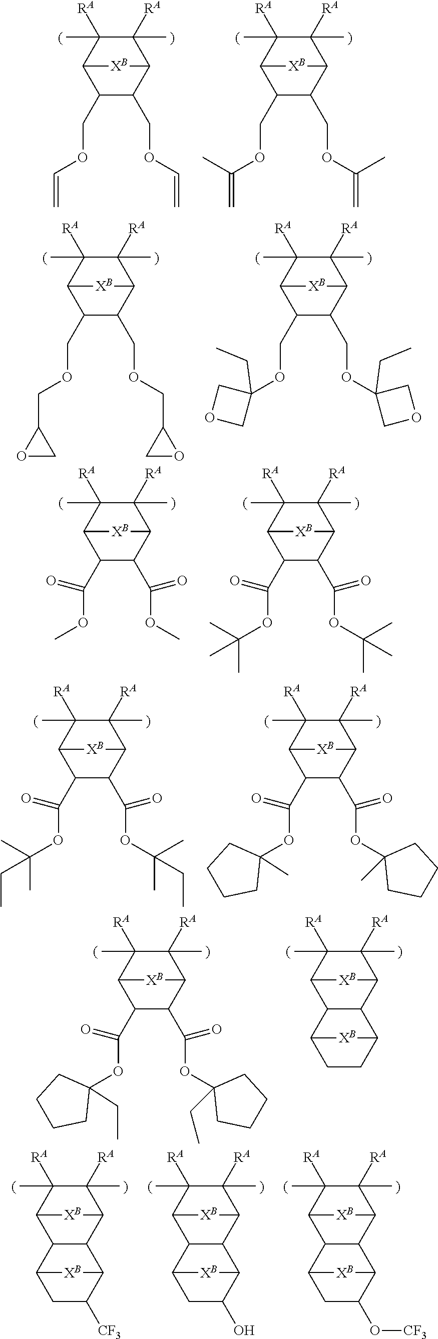

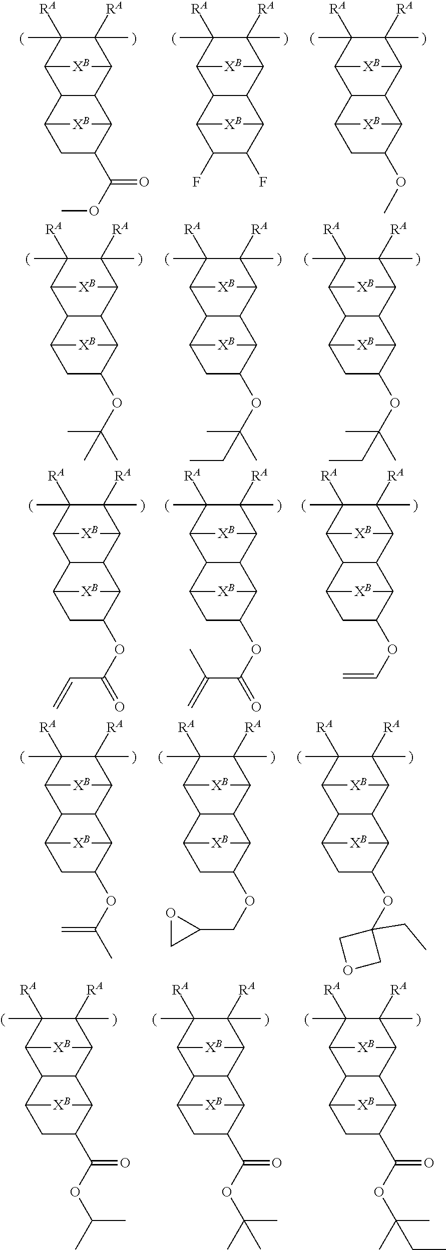

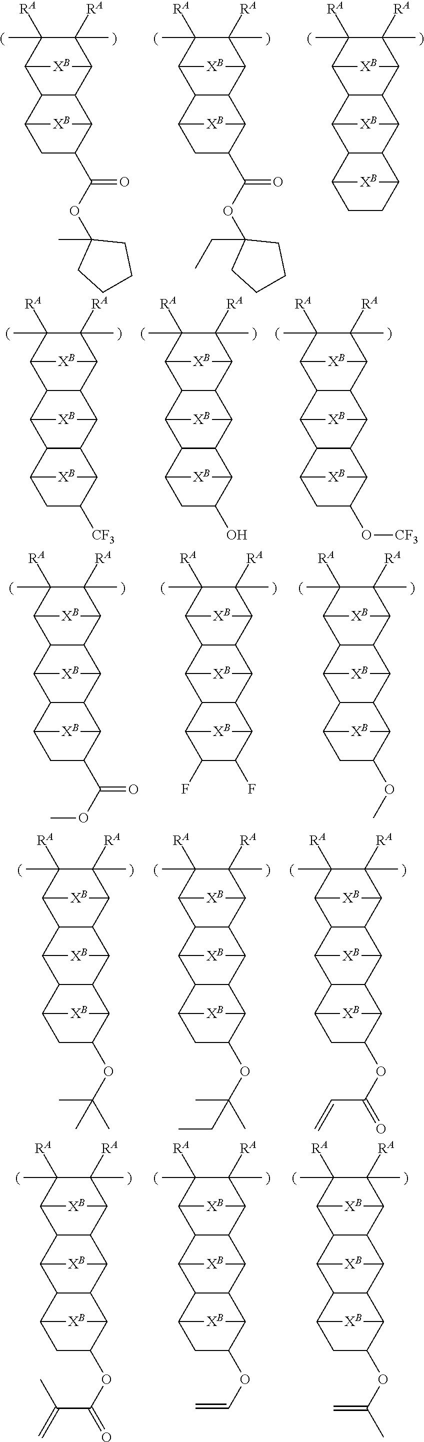

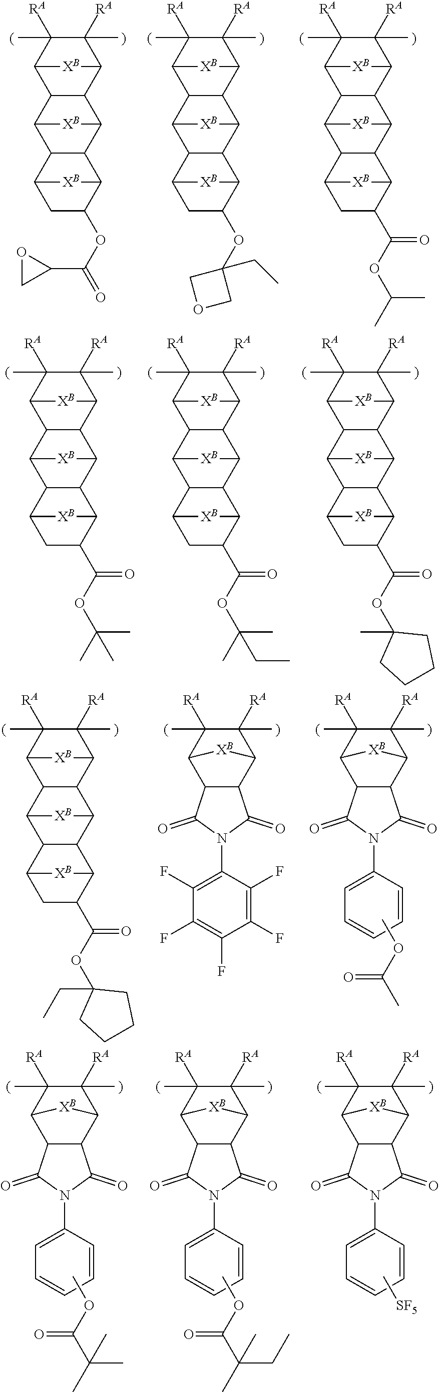

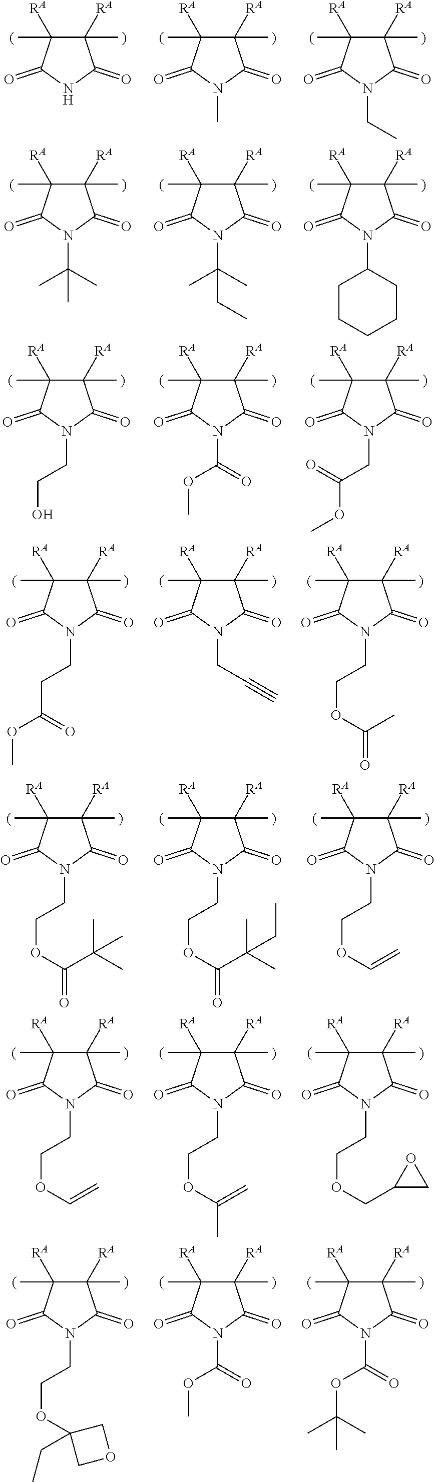

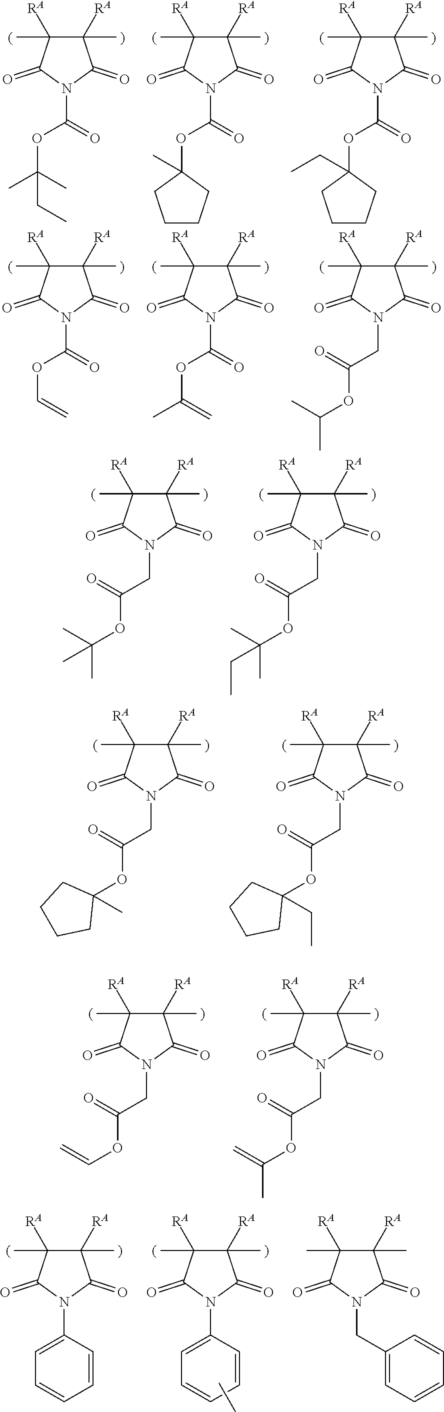

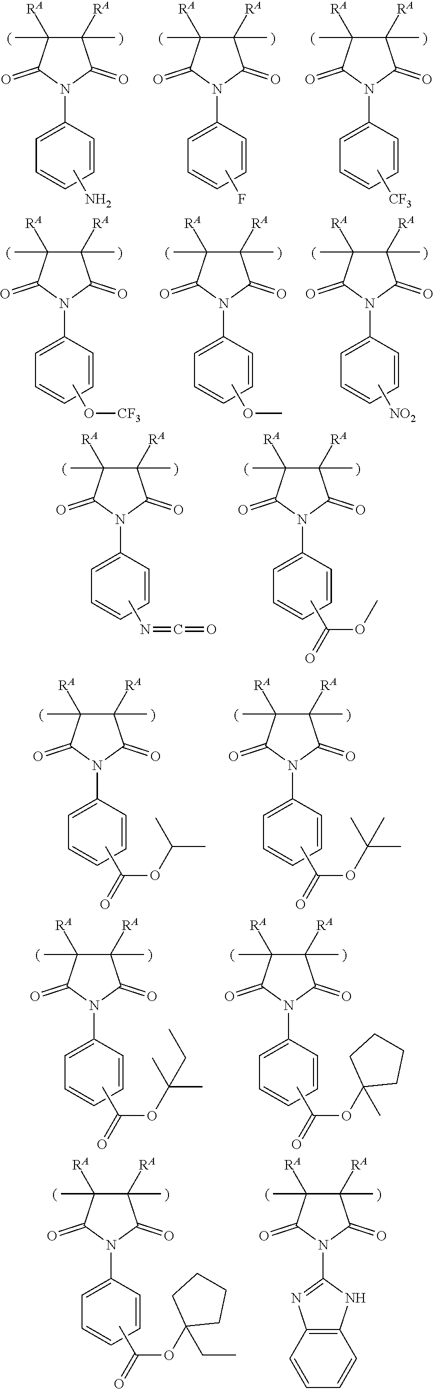

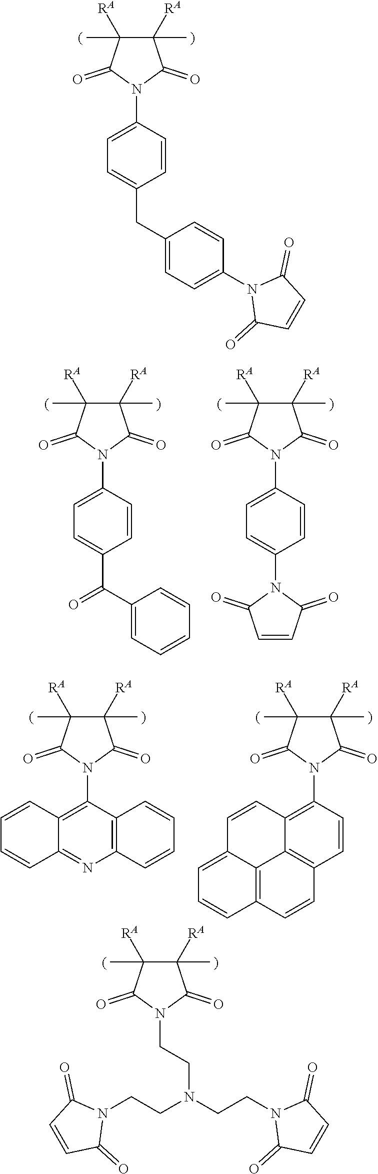

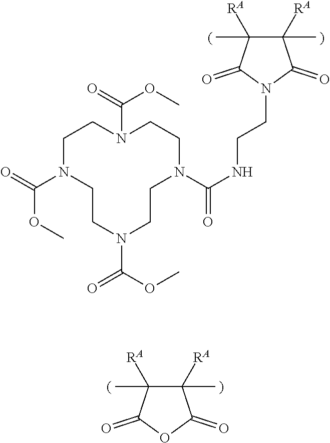

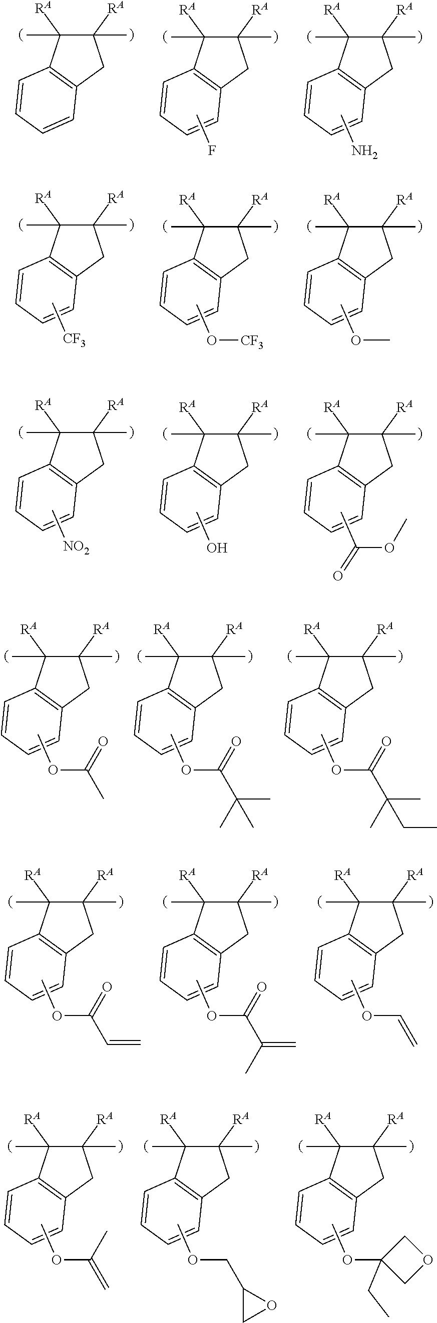

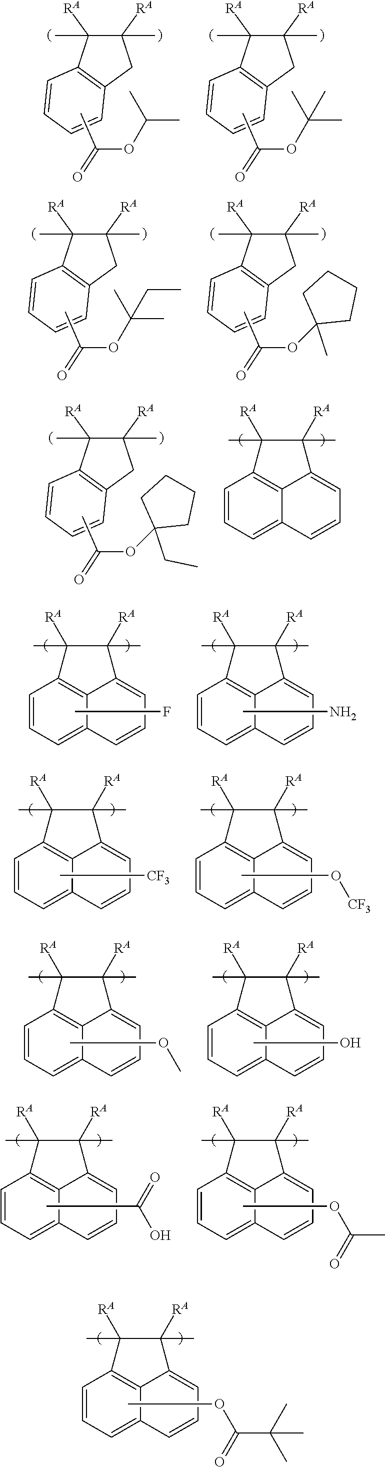

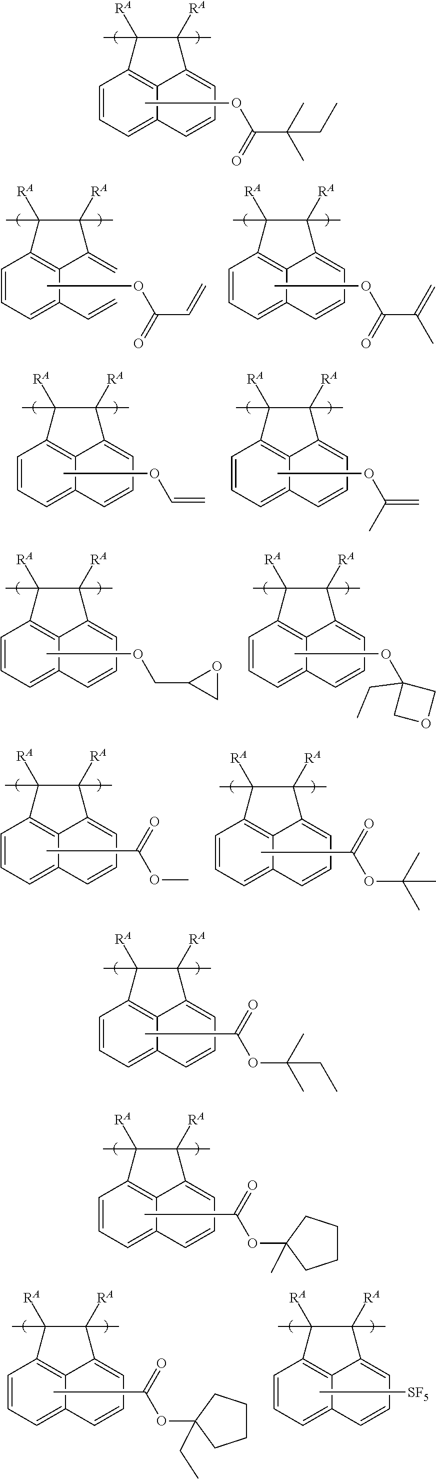

In the inventive resist composition, the content ratio of the hypervalent iodine compound to the carboxy-group-containing compound (the polymer including the repeating unit represented by the formula (4) and/or the compound represented by the formula (5)) (when the carboxy-group-containing compound is a carboxy-group-containing polymer, the content ratio of the hypervalent iodine compound (mol) to the carboxylic-acid-containing repeating unit (mol) in the polymer, and when the carboxy-group-containing compound is a monomolecular compound represented by the formula (5), the content ratio of the hypervalent iodine compound (mol) to the monomolecular compound (mol)) is preferably “hypervalent iodine compound”:“carboxy-group-containing compound=10:90 to 90:10, more preferably 20:80 to 80:20, and further preferably 30:70 to 70:30 in molar ratio. One kind of the hypervalent iodine compound may be used, or two or more kinds thereof may be used in combination. One kind of the carboxy-group-containing polymer may be used, or two or more kinds thereof having different composition ratios, weight-average molecular weights (Mw), and/or molecular weight distributions (Mw/Mn) may be used in combination. One kind of the monomolecular compound may be used, or two or more kinds thereof may be used in combination. One of the carboxy-group-containing polymer and the monomolecular compound may be used, or both may be used in combination.

In the carboxy-group-containing polymer, the content ratio (molar ratio) of the carboxy-group-containing repeating unit to the other repeating units is preferably “carboxy-group-containing repeating unit”:“other repeating units”=10:90 to 90:10, more preferably 15:85 to 85:15, and further preferably 20:80 to 80:20.

The carboxy-group-containing polymer preferably has a weight-average molecular weight (Mw) of 1000 to 500000, more preferably 3000 to 100000. Note that, in the present invention, Mw and number-average molecular weight Mn are values measured in terms of standard polystyrene by gel permeation chromatography (GPC) using tetrahydrofuran (THF) as an eluent, and dispersity Mw/Mn is a value calculated therefrom.

Furthermore, if the carboxy-group-containing polymer has a wide molecular weight distribution (Mw/Mn), polymers having a lower molecular weight or a higher molecular weight are present, and therefore, there are risks that foreign substances may be found on the pattern after exposure, and the pattern shape may be degraded. Accordingly, as pattern rule is miniaturized, the influence of Mw and Mw/Mn is likely to be greater, and therefore, to obtain a resist composition that can be used suitably for a fine pattern size, the carboxy-group-containing polymer preferably has a narrow dispersity Mw/Mn of 1.0 to 2.0.

Examples of methods for synthesizing the carboxy-group-containing polymer include a method of polymerizing a monomer to give a repeating unit described above by heating in an organic solvent in the presence of a radical polymerization initiator.

Specific examples of the organic solvent to be used in the polymerization reaction include toluene, benzene, THE, diethyl ether, dioxane, cyclohexane, cyclopentane, cyclopentanone, cyclohexanone, methyl ethyl ketone (MEK), propylene glycol monomethyl ether (PGME), propylene glycol monomethyl ether acetate (PGMEA), and γ-butyrolactone (GBL). Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of the polymerization initiator to be added is preferably 0.01 to 25 mol % of the total amount of the monomers to be polymerized. The reaction temperature is preferably 50 to 150° C., more preferably 60 to 100° C. The reaction time is preferably 2 to 24 hours, and from the viewpoint of production efficiency, more preferably 2 to 12 hours.

The polymerization initiator may be added to the solution of the monomer and supplied to the reaction vessel, or a solution of the initiator may be prepared separately from the solution of the monomer, and each may be supplied to the reaction vessel independently. There is a possibility that the polymerization reaction may progress due to radicals generated from the initiator during waiting time and an ultra-high molecular weight polymer may be generated, and therefore, from the viewpoint of quality control, it is preferable to prepare each of the monomer solution and the initiator solution independently and add the solutions dropwise. Furthermore, to adjust the molecular weight, a known chain transfer agent, such as dodecyl mercaptan and 2-mercaptoethanol may also be used. In this case, the amount of the chain transfer agent to be added is preferably 0.01 to 20 mol % of the total amount of the monomers to be polymerized.

Note that the amount of each monomer in the monomer solution can be, for example, set appropriately to achieve the preferable content ratios of the above-described repeating units.

### [Solvent]

The inventive resist composition contains a solvent. The solvent is not particularly limited as long as the solvent dissolves the hypervalent iodine compound, the carboxy-group-containing compound, and other components described later and allows film formation. As such a solvent, organic solvents are preferable, and specific examples thereof include: ketones, such as cyclohexanone, methyl-2-n-pentyl ketone, and methyl isoamyl ketone; alcohols, such as 3-methoxybutanol, 3-methyl-3-methoxybutanol, 1-methoxy-2-propanol, 1-ethoxy-2-propanol, diacetone alcohol, 4-methyl-2-pentanol, and methyl 2-hydroxyisobutyrate; ethers, such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters, such as propylene glycol monomethyl ether acetate, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; carboxylic acids, such as formic acid, acetic acid, and propionic acid; lactones, such as γ-butyrolactone; and mixed solvents thereof.

In the inventive resist composition, the amount of the solvent contained is preferably such an amount that the concentration of the solid contents in the resist composition is 0.1 to 20 mass %, more preferably 0.1 to 15 mass %, and further preferably 0.1 to 10 mass %. Note that, in the present invention, solid contents is a general term for the components other than the solvent out of all the components of the resist composition. One kind of the solvent may be used, or two or more kinds thereof may be used in mixture.

When the solvent is a combination of two or more kinds, the combination is not particularly limited, but an aprotic solvent and a protic solvent can be combined. An aprotic solvent can be selected from the above-mentioned ketones, ethers, esters, and lactones, and a protic solvent can be selected from the above-mentioned alcohols and carboxylic acids. A combination of an ester as an aprotic solvent and a carboxylic acid as a protic solvent is preferable. When an aprotic solvent and a protic solvent are used in combination, the mixing ratio is not particularly limited, but amounts where the aprotic solvent is 50 to 90 mass % of the total mass of the two are preferable, and amounts where the aprotic solvent is 60 to 80 mass % are more preferable. When the aprotic solvent is in the above range, the balance between the aprotic solvent and the protic solvent is suitable, and the solubility of the solid contents in the resist composition is favorable, so that a uniform resist composition can be achieved. In addition, the protic solvent can cooperate with the hypervalent iodine compound to improve the resolution of the resist composition.

### [Other Components]

The resist composition may further contain a surfactant. As the surfactant, a fluorine-based and/or silicone-based surfactant is preferable. Examples of such a surfactant include surfactants disclosed in paragraph [0276] of US2008/0248425A1. Furthermore, it is also possible to use a surfactant disclosed in paragraph [0280] of US2008/0248425A1, other than the fluorine-based and/or silicone-based surfactants.

When the resist composition contains the surfactant, the contained amount is preferably 0.0001 to 2 mass % of all the solid contents. One kind of the surfactant may be used, or two or more kinds thereof may be used in combination.

The resist composition may further contain a radical scavenger. When a radical scavenger is contained, the photoreaction during photolithography can be controlled, and sensitivity can be adjusted.

Examples of the radical scavenger include hindered phenols, quinones, hindered amines, and thiol compounds. Specifically, examples of the hindered phenols include dibutylhydroxytoluene (BHT) and 2,2′-methylenebis(4-methyl-6-tert-butylphenol). Examples of the quinones include 4-methoxyphenol (methoquinone) and hydroquinone. Examples of the hindered amines include 2,2,6,6-tetramethylpiperidine and 2,2,6,6-tetramethylpiperidine-N-oxy radical. Examples of the thiols include dodecanethiol and hexadecanethiol.

When the resist composition contains the radical scavenger, the contained amount is preferably 0.01 to 10 mass % of all the solid contents. One kind of the radical scavenger may be used, or two or more kinds thereof may be used in combination.

The resist composition may further contain a crosslinking agent. When a crosslinking agent is contained, the crosslinking reaction during photolithography can be promoted, the glass transition temperature of the pattern can be enhanced, and a pattern that is excellent in the resolution of a thin line can be obtained.

Examples of the crosslinking agent include compounds having a carbon-carbon unsaturated bond as a functional group, such as a vinyl group, a (meth)acrylate group, an allyl group, an alkynyl group, and an aromatic ring. Specifically, examples of compounds having a vinyl group include chain alkenes, branched alkenes, and cyclic alkenes, each optionally having a substituent. Examples of compounds having a (meth)acrylate group include acrylic acid, methacrylic acid, acrylic acid ester, and methacrylic acid ester, each optionally having a substituent. Examples of compounds having an allyl group include allyl alcohol, allyl ether, allyl ester, allyl amide, allylamine, and allyl-group-containing isocyanurates, each optionally having a substituent. Examples of compounds having an alkynyl group include chain alkynes, branched alkynes, cyclic alkynes, alkynyl alcohols, alkynyl ethers, alkynyl esters, alkynyl amides, alkynyl amines, and alkynyl-group-containing isocyanurates, each optionally having a substituent. Examples of compounds having an aromatic ring include arenes, heteroarenes, styrene, stilbene, phenylacetylene, acenaphthylene, and chalcone, each optionally having a substituent. The crosslinking agent may have only one of the functional groups, or may have a plurality of the groups. The number of the functional groups contained in the crosslinking agent is preferably 1 or more and 10 or less, more preferably 2 or more and 8 or less.

When the resist composition contains the crosslinking agent, the contained amount is preferably 0.01 to 50 mass % of all the solid contents. One kind of the crosslinking agent may be used, or two or more kinds thereof may be used in combination.

When the resist composition contains the crosslinking agent, a photo-polymerization initiator (photo-radical generator) may further be contained. The photo-polymerization initiator generates radicals upon irradiation with a high-energy beam, and can promote the crosslinking of the crosslinking agent.

Specific examples of the photo-polymerization initiator include: benzophenone derivatives, such as benzophenone, methyl O-benzoylbenzoate, 4-benzoyl-4′-methyldiphenyl ketone, dibenzyl ketone, and fluorenone; acetophenone derivatives, such as 2,2′-diethoxyacetophenone, 2-hydroxy-2-methylpropiophenone, 2,2-dimethoxy-1,2-diphenylethan-1-one, 1-hydroxycyclohexyl phenyl ketone, 2-methyl-1-[4-(methylthio)phenyl]-2-morpholinopropan-1-one, 2-hydroxy-1-{4-[4-(2-hydroxy-2-methylpropionyl)-benzyl]-phenyl}-2-methylpropan-1-one, and methyl phenylglyoxylate; thioxanthone derivatives, such as thioxanthone, 2-methylthioxanthone, 2-isopropylthioxanthone, 4-isopropylthioxanthone, 2-chlorothioxanthone, and diethylthioxanthone; benzyl derivatives, such as benzyl, benzyldimethyl ketal, and benzyl-β-methoxyethyl acetal; benzoin derivatives, such as benzoin, benzoin methyl ether, and 2-hydroxy-2-methyl-1-phenylpropan-1-one; oxime compounds, such as 1-phenyl-1,2-butanedione-2-(O-methoxycarbonyl) oxime, 1-phenyl-1,2-propanedione-2-(0-methoxycarbonyl) oxime, 1-phenyl-1,2-propanedione-2-(0-ethoxycarbonyl) oxime, 1-phenyl-1,2-propanedione-2-(0-benzoyl) oxime, 1,3-diphenylpropanetrione-2-(O-ethoxycarbonyl) oxime, 1-phenyl-3-ethoxypropantrione-2-(O-benzoyl) oxime, 1,2-octanedione-1-[4-(phenylthio)-2-(0-benzoyloxime)], ethenone-1-[9-ethyl-6-(2-methylbenzoyl)-9H-carbazol-3-yl]-1-(O-acetyloxime); x-hydroxyketone compounds, such as 2-hydroxy-2-methyl-1-phenylpropan-1-one, 1-[4-(2-hydroxyethoxy)phenyl]-2-hydroxy-2-methyl-1-propan-1-one, and 2-hydroxy-1-{4-[4-(2-hydroxy-2-methylpropionyl)-benzyl]phenyl}-2-methylpropane; x-aminoalkylphenone compounds, such as 2-benzyl-2-dimethylamino-1-(4-morpholinophenyl)-butanone-1 and 2-dimethylamino-2-(4-methylbenzyl)-1-(4-morpholin-4-yl-phenyl) butan-1-one; phosphine oxide compounds, such as bis(2,4,6-trimethylbenzoyl)-phenylphosphine oxide, bis(2,6-dimethoxybenzoyl)-2,4,4-trimethyl-pentylphosphine oxide, and 2,4,6-trimethylbenzoyldiphenylphosphine oxide; and titanocene compounds, such as bis(n5-2,4-cyclopentadien-1-yl)-bis(2,6-difluoro-3-(1H-pyrrol-1-yl)phenyl) titanium.

When the resist composition contains the photo-polymerization initiator, the contained amount is preferably 0.1 to 10 mass %, more preferably 0.1 to 5 mass %, and most preferably 0.1 to 1 mass % of all the solid contents. When the amount is 0.1 mass % or more, a blending effect can be achieved sufficiently.

The resist composition mainly contains a hypervalent iodine compound and a carboxy-group-containing compound as described above, and polymers containing acid-labile groups and photo-acid generators, which are contained in conventional chemically amplified resist compositions, are not essential. However, the inventive resist composition makes it possible to form a positive pattern, where exposed portions become soluble in a developer, or a negative pattern, where exposed portions become insoluble in a developer, especially by exposure to EB or EUV. The mechanism is not completely clear, but the following conjecture can be made, for example.

The hypervalent iodine compound represented by the formula (1), (2), or (3), being a compound in which a carboxylate is coordinated to the hypervalent iodine, is a compound having a tricoordinate hypervalent iodine having a carboxylate ligand. It can be assumed that when such a tricoordinate iodine compound is mixed with a carboxy-group-containing compound, exchange with the carboxylate ligand occurs by an equilibrium reaction. In this event, if the original carboxylate ligand can be removed by some method, a hypervalent iodine compound having a new ligand is generated. For example, when 3-(diacetoxyiodo)thiophene, which is a hypervalent iodine compound, and a carboxy-group-containing compound are mixed together and the generated acetic acid, having a low boiling point, is removed, ligand exchange is completed. Here, a polymer in which the carboxy-group-containing compound is crosslinked with the hypervalent iodine compound is obtained.

The polymer crosslinked with the hypervalent iodine compound is generated at the time of film formation. This is because such a crosslinked polymer is insoluble in most organic solvents, and therefore, a solution cannot be prepared even if the polymer is synthesized beforehand. This is conjectured to be because the hypervalent iodine compound, originally having low solvent solubility due to high polarization, contains the carboxy-group-containing compound as a ligand, and thus, solubility is even more degraded. Accordingly, in this step, it is desirable to remove the original low-molecular-weight carboxylic acid component at the time of film formation and in the subsequent baking process, thus completing the ligand exchange reaction and also forming a resist film.

The resist film obtained from the inventive resist composition changes in polarity by decomposing of the hypervalent iodine compound by light, being the main component of the resist film, and a pattern is formed by a development process. The mechanism is not completely clear, but the following conjecture can be made, for example. Incidentally, by selecting the developer appropriately, a positive or negative pattern can be formed.

The inventive resist composition can be either a positive type or a negative type depending on the choice of components. In the case of a positive type, a polymer in which a hypervalent iodine compound is bonded is contained at the time of film formation. By the polymer being decomposed due to light, a monovalent iodine compound is formed, and at the same time, the bond between the carboxy-group-containing compound and the hypervalent iodine compound is removed, and the molecular weight is reduced. It is conjectured that, as a result, a positive pattern, where exposed portions are removed with an organic solvent, is formed.

On the other hand, in the case of a negative type, a polymer generated at the time of film formation, crosslinked with a hypervalent iodine compound, is contained. By the polymer being decomposed due to light, exchange of cross links or bonds occurs, and increase in molecular weight and polarity conversion occur. It is conjectured that, as a result, a negative pattern, where unexposed portions are removed with an aqueous alkaline solution, is formed.

In a hypervalent iodine compound represented by the formula (1), (2), or (3), one of a thiophene, pyrazole, or pyridine skeleton is bonded to an iodine atom. Accordingly, unlike a tricoordinate hypervalent iodine compound having an aryl group and a carboxylate ligand, the hypervalent iodine compound has a small molecular weight, is excellent in solubility in developers, and is also conjectured to have a greatly different photoreactivity, and therefore, makes it possible to form a pattern having low roughness and excellent resolution with high sensitivity.

From the above-described conjecture, it can be said that the inventive resist composition is a non-chemically amplified resist composition. The inventive resist composition does not require an acid-labile group-containing polymer or a photo-acid generator, unlike conventional chemically amplified resist compositions. Therefore, adverse effects (e. g. image blurs) due to acid diffusion do not occur, and resolution of a fine pattern is possible.

The inventive resist composition is extremely effective, especially in EUV lithography. This results from iodine atoms, which are capable of greatly absorbing EUV light, being contained. That is, shot noise can be reduced, and higher resolution and lower LWR can be achieved.

As a resist composition for EUV lithography with which a fine pattern can be formed, a metal resist that mainly contains a compound of tin, which is a metal having a high absorbance of EUV light in the same manner as iodine atoms (e. g. Patent Document 2) is reported. However, as described above, such a metal resist has many issues such as insufficient solubility in a solvent, storage stability, and defects due to residues after etching caused by a metal element being contained. On the other hand, the inventive resist composition has an advantage over metal resists regarding defects, since a metal element is not used, and there are no problems regarding solubility in a solvent either. Moreover, the inventive resist composition is applicable in the case of either a positive type or a negative type, and therefore, has a wide range of uses. For example, in a contact hole formation process, a reversal process step is necessary after forming a pillar pattern in the case of a metal resist performed with negative development, but such a step is unnecessary in the case of a positive resist. Therefore, it can be said that the inventive resist composition is more useful than metal resists from the viewpoint of the simplicity and convenience of the process as well.

JP2015-180928A and JP2018-95853A disclose a resist composition containing a hypervalent iodine compound as an additive and a resist composition in which a hypervalent iodine compound is incorporated in the polymer skeleton of a base polymer. However, the only characteristic of the resist compositions disclosed in these Patent Documents is improvement of line edge roughness, and there is no mention whatsoever regarding the possibility that the hypervalent iodine compound may be photolysed, or the possibility that resist composition may function as a material for a non-chemically amplified resist composition. Furthermore, according to the description regarding the contained amount and specific examples, the hypervalent iodine compound is not the main component. Accordingly, it is considered that a material that makes it possible to reduce shot noise in EUV lithography and also form a fine pattern as a non-chemically amplified resist composition material like the material of the present invention cannot be conceived from these Patent Documents. That is, it can be said that the present invention provides a clearly novel resist composition and a clearly novel patterning process.

### [Laminate]

The present invention provides a laminate including: a substrate; and a resist film, which is a film body formed of the above-described resist composition, on the substrate. In such a laminate, including a resist film obtained from the non-chemically amplified resist composition of the present invention, the resist film, which is a film body of the above-described resist composition, has extremely high sensitivity, also exhibits excellent resolution, is extremely effective for precise fine processing, and in addition, is applicable to either positive or negative patterning. Therefore, the laminate has a wide range of uses, and is extremely highly useful in resist process technology.

In this case, a resist underlayer film can be further provided as necessary between the substrate and the resist film.

Furthermore, in the inventive laminate, the resist film preferably contains a product made by a ligand exchange reaction of the hypervalent iodine compound and the carboxy-group-containing compound. That is, the laminate which is formed by a substrate and a resist film obtained from the inventive resist composition on the substrate is obtained and the resist film is preferably one formed by ligand exchange between the hypervalent iodine compound and the carboxy-group-containing compound.

As described above, by removing by-product low-molecular-weight carboxylic acid produced during film formation and in the subsequent baking process, the ligand exchange reaction between the hypervalent iodine compound and the carboxy-group-containing compound progresses, and a resist film containing a ligand exchange reaction product is formed (that is, a film body is produced). By the ligand exchange being completed, a polymer in which the carboxy-group-containing compound is crosslinked with the hypervalent iodine compound is obtained. It is preferable to form the resist film on completing the ligand exchange reaction in this manner.

### [Patterning Process]

When the inventive resist composition is used for manufacturing various integrated circuits, a known lithography technique can be applied. Examples of patterning processes include a method including the steps of: forming a resist film by using the above-described resist composition on a substrate or on a resist underlayer film of a substrate on which the resist underlayer film has been laminated; exposing the resist film by using a high-energy beam; and developing the exposed resist film by using a developer. Hereinafter, the resist underlayer film is also simply referred to as an “underlayer film”.

Firstly, the inventive resist composition is applied onto a substrate for manufacturing an integrated circuit, on an underlayer film of a substrate (Si, SiO₂, SiN, SiON, TiN, WSi, BPSG, SOG, organic antireflective film, etc.) on which the underlayer film has been laminated, on a substrate for manufacturing a mask circuit, or on an underlayer film of a substrate (Cr, Cro, CrON, MoSi₂, SiO₂, etc.) on which the underlayer film has been laminated, by an appropriate coating process, such as spin coating, roll coating, flow coating, dip coating, spray coating, or doctor coating, so that the thickness of the coating film is 0.01 to 2 μm. The resultant is prebaked on a hot plate preferably at 60 to 200° C. for 10 seconds to 30 minutes, more preferably 80 to 180° C. for 30 seconds to 20 minutes. Thus, a resist film is formed. Note that an underlayer film means a film formed between the substrate and the resist film in a multilayer resist process. The underlayer film is not particularly limited, and a conventionally known film can be used.

Subsequently, the resist film is exposed by using a high-energy beam. Examples of the high-energy beam include ultraviolet ray (g-line (436 nm), h-line (405 nm), i-line (365 nm), etc.), deep ultraviolet ray, EB, EUV, X-ray, soft X-ray, excimer laser beam (KrF excimer laser beam, ArF excimer laser beam, etc.), γ-ray, and synchrotron radiation. As the high-energy beam, it is preferable to use an i-line, a KrF excimer laser beam, an ArF excimer laser beam, an electron beam, or an extreme ultraviolet ray. When ultraviolet ray, deep ultraviolet ray, EUV, X-ray, soft X-ray, excimer laser beam, γ-ray, synchrotron radiation, or the like is employed as the high-energy beam, the irradiation is performed directly or while using a mask for forming a target pattern at an exposure dose of preferably about 1 to 300 mJ/cm², more preferably about 10 to 200 mJ/cm². When an EB is employed as the high-energy beam, the writing is performed directly or while using a mask for forming a target pattern at an exposure dose of preferably about 0.1 to 8000 μC/cm², more preferably about 0.5 to 5000 μC/cm². Note that the inventive resist composition is particularly suitable for fine patterning with an EB or EUV, among the high-energy beams.

After the exposure, PEB is performed as necessary. In this event, the PEB is preferably performed after the exposure on a hot plate or in an oven under the conditions of 30 to 200° C. for 10 seconds to 30 minutes, more preferably 60 to 180° C. for 30 seconds to 20 minutes.

After the exposure or after the PEB, development is performed by using a developer to perform patterning. Examples of the developer used in this event include: aqueous alkaline solutions, such as an aqueous solution of tetramethylammonium hydroxide and an aqueous solution of tetrabutylammonium hydroxide; and organic solvents, such as 2-octanone, 2-nonanone, 2-heptanone, 3-heptanone, 4-heptanone, 2-hexanone, 3-hexanone, diisobutyl ketone, 5-methyl-2-hexanone, methylcyclohexanone, acetophenone, methylacetophenone, isopropyl alcohol, isoamyl alcohol, n-butanol, tert-butyl alcohol, tert-pentyl alcohol, n-pentanol, cyclohexanol, formic acid, acetic acid, propionic acid, propyl acetate, butyl acetate, isobutyl acetate, pentyl acetate, butenyl acetate, isopentyl acetate, cyclohexyl acetate, 4-tert-butylcyclohexyl acetate, octyl acetate, isobornyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, phenylmethyl acetate, phenylethyl acetate, benzyl formate, phenylethyl formate, methyl 3-phenylpropionate, benzyl propionate, 2-phenylethyl acetate, 1-propanol, 3-methoxybutanol, 3-methyl-3-methoxybutanol, 1-methoxy-2-propanol, 1-ethoxy-2-propanol, 3-methyl-1-butanol, diacetone alcohol, 4-methyl-2-pentanol, 3-methylcyclohexanol, 3,5,5-trimethylhexyl alcohol, 2,6-dimethyl-4-heptanol, toluene, anisole, and ε-caprolactone. One kind of these developers may be used, or two or more kinds thereof may be used in mixture.

After the development, rinsing is performed as necessary. The rinsing liquid is preferably a solvent that is miscible with the developer but does not dissolve the resist film. As such a solvent, it is preferable to use an alcohol having 3 to 10 carbon atoms, an ether compound having 8 to 12 carbon atoms, and an alkane, alkene, alkyne, and aromatic solvent, each having 6 to 12 carbon atoms.

The rinsing can reduce resist pattern collapse and defect formation. Meanwhile, the rinsing is not necessarily essential, and the amount of the solvent used can be reduced by not performing the rinsing.

In the inventive resist composition, a difference occurs in the solubility between exposed portions and unexposed portions by virtue of exposure as described above, and a positive or negative pattern can be formed. Therefore, it is possible to use a developer that dissolves exposed portions and does not dissolve unexposed portions, or a developer that dissolves unexposed portions and does not dissolve exposed portions. Thus, the inventive patterning process makes it possible to form a positive or negative pattern by appropriately selecting a developer, and therefore, is widely applicable to various kinds of fine patterning.

As described, the present invention is related to non-chemically amplified resist compositions, and a characteristic thereof is that a heterocycle-containing hypervalent iodine compound is contained as a resist material containing a hypervalent iodine compound. By solvent solubility being enhanced by virtue of this characteristic, sensitivity can be improved, and in addition, uniformity is enhanced, so that roughness is reduced. Therefore, the present invention is effective for fine patterning.

### EXAMPLES

Hereinafter, the present invention will be specifically described with reference to Synthesis Examples, Examples, and Comparative Examples. However, the present invention is not limited to the following Examples.

### [1] Synthesis of Hypervalent Iodine Compound [Synthesis Example 1-1] Synthesis of Hypervalent Iodine Compound I-1

3-iodothiophene (4.2 g, 20.0 mmol) was dispersed in acetic acid (200 mL), 10 equivalents of sodium perborate tetrahydrate (30.8 g, 200 mmol) was added thereto, and the mixture was stirred at 40° C. for 24 hours. After the reaction was completed, the solvent was removed under reduced pressure, 100 ml of water and 150 mL of dichloromethane were added thereto, and extraction was performed. The organic layer was washed three times with 50 mL of water, the solvent was distilled off under reduced pressure, 100 mL of n-hexane was added thereto, the mixture was stirred under room temperature for 30 minutes, and a solid was collected by filtration. The obtained solid was dried at 40° C. to obtain I-1 as a crystal (5.91 g, 90% yield).

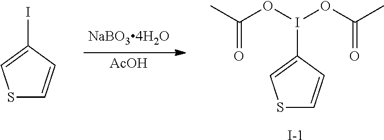

The nuclear magnetic resonance spectrum was as follows.

¹H NMR (500 MHz, CDCl₃): δ=2.02 (s, 6H), 7.48 (dd, J=3.0, 5.2 Hz, 1H), 7.52 (dd, J=1.3, 5.2 Hz, 1H), 8.05 (dd, J=1.3, 3.0 Hz, 1H).

### [Synthesis Example 1-2] Synthesis of Hypervalent Iodine Compound I-2

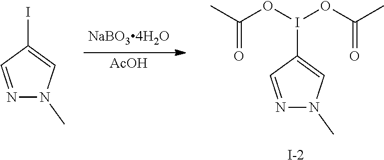

I-2 was synthesized in the same manner as I-1, except that the raw materials were changed as in the above formula. (92% yield)

The nuclear magnetic resonance spectrum was as follows.

¹H NMR (500 MHz, CDCl₃): δ=2.00 (s, 6H), 4.02 (s, 3H), 7.86 (d, J=0.6 Hz, 1H), 7.92 (s, 1H).

### [Synthesis Example 1-3] Synthesis of Hypervalent Iodine Compound I-3

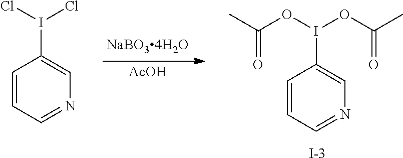

I-3 was synthesized in the same manner as I-1, except that the raw materials were changed as in the above formula. (78% yield)

The nuclear magnetic resonance spectrum was as follows.

¹H NMR (500 MHz, CDCl₃): δ=2.03 (s, 6H), 7.48 (d, J=4.0 Hz, 1H), 8.42 (dt, J=4.2 Hz, 1H), 8.85 (d, J=4.0 Hz, 1H), 9.17 (d, J=2.0 Hz, 1H).

### [2] Synthesis of Carboxy-Group-Containing Polymer

The monomers a-1 to a-3, b-1 to b-3, and c-1 to c-3 used for the synthesis of carboxy-group-containing polymers are as follows.

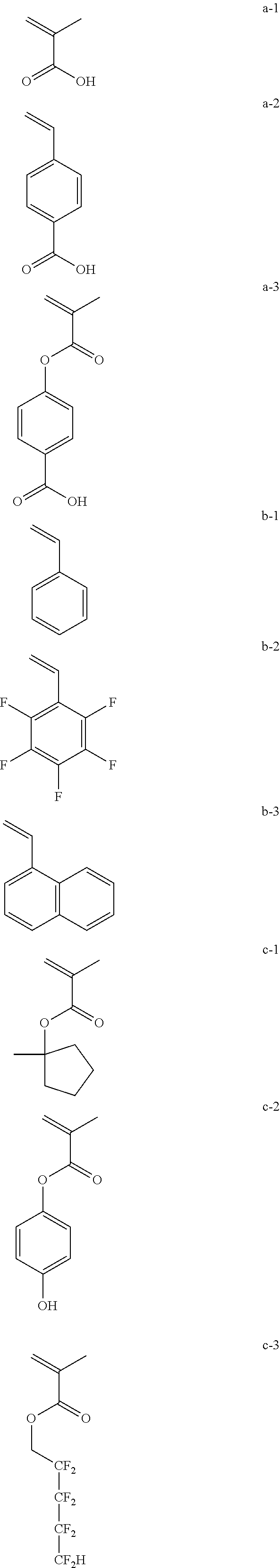

### [Synthesis Example 2-1] Synthesis of Polymer P-1

Under a nitrogen atmosphere, monomer a-1 (56 g), monomer b-1 (36 g), 5.4 g of V-601 (manufactured by FUJIFILM Wako Pure Chemical Corporation), and 180 g of MEK were added into a flask to prepare a monomer-polymerization initiator solution. Into another flask with a nitrogen atmosphere, 55 g of MEK was added and heated to 80° C. with stirring, and then the monomer-polymerization initiator solution was added dropwise over 4 hours. After the dropwise addition, the polymerization liquid was further stirred for 2 hours with maintaining the temperature at 80° C., and then cooled to room temperature. The obtained polymerization liquid was added dropwise to 4000 g of vigorously stirred hexane, and a precipitated polymer was filtered. The obtained polymer was washed twice with hexane (1200 g), and then dried in vacuo at 50° C. for 20 hours to obtain a white powder polymer P-1 (90 g, 98% yield). The polymer P-1 had Mw of 8000 and Mw/Mn of 1.42. The Mw and Mn are standard polystyrene-converted measurement values obtained by GPC using THE as an eluent. Specifically, measurement was carried out under the following conditions (the same hereinafter).
Apparatus: HLC-8320GPC
Column: TSK guardcolumn
   TSKgel G4000HXL
   TSKgel G2000HXL
   TSKgel superH5000
Constant temperature of pump and column: 40° C.
Eluent: THE
Detector: RI (refractive index) detector
Injection volume: 100 μl

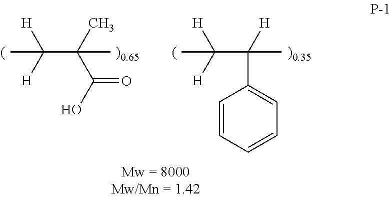

### [Synthesis Examples 2-2 to 2-13] Synthesis of Polymers P-2 to P-13

The polymers shown in Table 1 below were synthesized in the same manner as in Synthesis Example 2-1 except that the kinds and blending ratios of the monomers were changed. Note that polymer P-13 does not have a carboxy group (—COOH), and is not a carboxy-group-containing compound of the present invention.

TABLE 1

| | | Introduction | | Introduction | | |
|---|---|---|---|---|---|---|
| | | rate | | rate | | |
| Polymer | Unit 1 | (mol %) | Unit 2 | (mol %) | Mw | Mw/Mn |
| P-1 | a-1 | 65 | b-1 | 35 | 8000 | 1.42 |
| P-2 | a-1 | 50 | b-2 | 50 | 8400 | 1.51 |
| P-3 | a-1 | 60 | b-3 | 40 | 8100 | 1.42 |
| P-4 | a-1 | 60 | c-3 | 40 | 8100 | 1.42 |
| P-5 | a-2 | 65 | b-1 | 35 | 8000 | 1.44 |
| P-6 | a-2 | 50 | b-2 | 50 | 8600 | 1.41 |
| P-7 | a-2 | 60 | b-3 | 40 | 7900 | 1.49 |
| P-8 | a-2 | 65 | c-3 | 35 | 9800 | 1.45 |
| P-9 | a-3 | 65 | b-1 | 35 | 9400 | 1.40 |
| P-10 | a-3 | 50 | b-2 | 50 | 9000 | 1.45 |
| P-11 | a-3 | 60 | b-3 | 40 | 7500 | 1.46 |
| P-12 | a-3 | 65 | c-3 | 35 | 7000 | 1.48 |
| P-13 | c-1 | 60 | c-2 | 40 | 8500 | 1.45 |

### [3] Preparation of Resist Compositions

### Examples 1-1 to 1-21 and Comparative Examples 1-1 to 1-4

A hypervalent iodine compound and a carboxy-group-containing compound were dissolved in a solvent containing 0.01 mass % of a surfactant (PF-636, manufactured by Omnova Solutions Inc.) in the constitution shown below in Table 2, and the obtained solution was filtered with a 0.2-μm Teflon (registered trademark) filter to prepare resist compositions (R-01 to R-21) of Examples 1-1 to 1-21 and resist compositions (CR-01 and CR-02) of Comparative Examples 1-1 and 1-2. Meanwhile, a polymer, a photo-acid generator, and a sensitivity modifier were dissolved in a solvent containing 0.01 mass % of a surfactant (PF-636, manufactured by Omnova Solutions Inc.) in the constitution shown below in Table 3, and the obtained solution was filtered with a 0.2-μm Teflon (registered trademark) filter to prepare resist compositions (CR-03 and CR-04) of Comparative Examples 1-3 and 1-4. Note that the resist composition R-04 of Example 1-4 contains a combination of a thiophene-type hypervalent iodine compound represented by the formula (1) and a pyrazole-type hypervalent iodine compound represented by the formula (2).

TABLE 2

| | | | Other | | | |
|---|---|---|---|---|---|---|
| | | Hypervalent | hypervalent | Carboxylic | | |
| | | iodine | iodine | acid | | |
| | | compound | compound | compound | Solvent 1 | Solvent 2 |
| | | (parts by | (parts by | (parts by | (parts by | (parts by |
| | Resist | mass) | mass) | mass) | mass) | mass) |
| Example 1-1 | R-01 | I-1 (10) | | P-1(9) | PGMEA | AcOH |
| | | | | | (800) | (200) |
| Example 1-2 | R-02 | I-2 (13) | | P-1(9) | PGMEA | AcOH |
| | | | | | (800) | (200) |
| Example 1-3 | R-03 | I-3 (11) | | P-1(9) | PGMEA | AcOH |
| | | | | | (800) | (200) |
| Example 1-4 | R-04 | I-1 (5) | I-2 (2) | P-1(9) | PGMEA | AcOH |
| | | | | | (800) | (200) |
| Example 1-5 | R-05 | I-2 (13) | | P-2 (17) | PGMEA | AcOH |
| | | | | | (800) | (200) |
| Example 1-6 | R-06 | I-2 (13) | | P-3 (11) | PGMEA | AcOH |
| | | | | | (800) | (200) |
| Example 1-7 | R-07 | I-2 (13) | | P-4 (17) | PGMEA | AcOH |
| | | | | | (800) | (200) |
| Example 1-8 | R-08 | I-2 (13) | | P-5 (12) | PGMEA | AcOH |
| | | | | | (800) | (200) |
| Example 1-9 | R-09 | I-2 (13) | | P-6 (21) | PGMEA | AcOH |
| | | | | | (800) | (200) |
| Example 1-10 | R-10 | I-2 (13) | | P-7 (15) | PGMEA | AcOH |
| | | | | | (800) | (200) |
| Example 1-11 | R-11 | I-2 (13) | | P-8 (19) | PGMEA | AcOH |
| | | | | | (800) | (200) |
| Example 1-12 | R-12 | I-2 (13) | | P-9 (21) | PGMEA | AcOH |
| | | | | | (800) | (200) |
| Example 1-13 | R-13 | I-2 (13) | | P-10 (29) | PGMEA | AcOH |
| | | | | | (800) | (200) |
| Example 1-14 | R-14 | I-2 (13) | | P-11 (24) | PGMEA | AcOH |
| | | | | | (800) | (200) |
| Example 1-15 | R-15 | I-2 (13) | | P-12 (27) | PGMEA | AcOH |
| | | | | | (800) | (200) |
| Example 1-16 | R-16 | I-2 (13) | | m-1 (7) | PGMEA | AcOH |
| | | | | | (800) | (200) |
| Example 1-17 | R-17 | I-2 (13) | | m-2 (4) | PGMEA | AcOH |
| | | | | | (800) | (200) |
| Example 1-18 | R-18 | I-2 (13) | | m-3 (7) | PGMEA | AcOH |
| | | | | | (800) | (200) |
| Example 1-19 | R-19 | I-2 (13) | | m-4 (7) | PGMEA | AcOH |
| | | | | | (800) | (200) |
| Example 1-20 | R-20 | I-2 (13) | | m-5 (10) | PGMEA | AcOH |
| | | | | | (800) | (200) |
| Example 1-21 | R-21 | I-2 (13) | | m-6 (8) | PGMEA | AcOH |
| | | | | | (800) | (200) |
| Comparative | CR-01 | I-4 (10) | | P-1(9) | PGMEA | AcOH |
| Example 1-1 | | | | | (800) | (200) |
| Comparative | CR-02 | I-4 (10) | | P-9 (12) | PGMEA | AcOH |
| Example 1-2 | | | | | (800) | (200) |

TABLE 3

| | | | Photo- | | | |
|---|---|---|---|---|---|---|
| | | | acid | Sensitivity | | |
| | | Polymer | generator | modifier | Solvent 1 | Solvent 2 |
| | Resist | (parts by | (parts by | (parts by | (parts by | (parts by |
| | composition | mass) | mass) | mass) | mass) | mass) |
| Comparative | CR-03 | P-13 (80) | PAG-1 | Q-1 (6) | PGMEA | GBL (210) |
| Example 1-3 | | | (19) | | (1890) | |
| Comparative | CR-04 | P-13 (80) | PAG-1 | I-4 (5) | PGMEA | GBL (210) |
| Example 1-4 | | | (19) | | (1890) | |

In Tables 2 and 3, hypervalent iodine compound I-4, carboxy-group-containing compounds m-1 to m-6, photo-acid generator PAG-1, sensitivity modifier Q-1, and solvents are as follows.

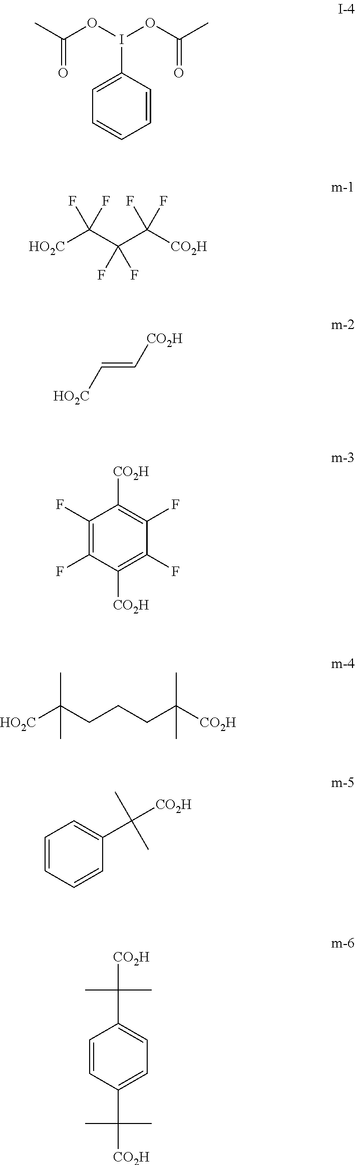

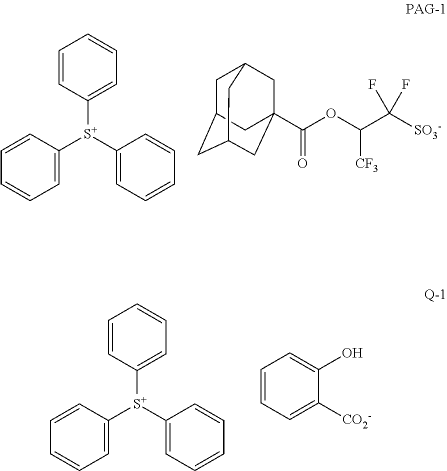

### Solvents:

PGMEA (propylene glycol monomethyl ether acetate)
AcOH (acetic acid)
GBL (γ-butyrolactone)

### [4] EUV Lithography Evaluation (Line-and-Space Pattern)

### Examples 2-1 to 2-21 and Comparative Examples 2-1 to 2-4

Each of the resist compositions (R-01 to R-21 and CR-01 to CR-04) was applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness, and subjected to post apply bake (PAB) by using a hot plate at the temperature shown in Table 4 for 60 seconds to form a resist film having a film thickness of 40 nm. The resist film was exposed using an EUV scanner NXE3400 (NA 0.33, σ 0.9, 90° dipole illumination), manufactured by ASML Holding N.V., to form a 36-nm 1:1 line-and-space (LS) pattern. Then, PEB was performed on a hot plate at the temperature shown in Table 4 for 60 seconds, and then development was performed with the developer shown in Table 4 for 30 seconds to form an LS pattern having a space width of 18 nm and a pitch of 36 nm.

Regarding the obtained resist pattern, the following evaluations were carried out. The results are shown in Table 4.

### [Sensitivity Evaluation]

The LS pattern was observed using a length-measurement SEM (CG-6300), manufactured by Hitachi High-Technologies Corporation, and an optimum exposure dose Eₒₚ (mJ/cm²) to yield the LS pattern with 18 nm in space width and 36 nm in pitch was determined to specify this value as sensitivity.

### [LWR Evaluation]

In the LS pattern obtained by irradiation at the optimum exposure dose, sizes in 10 positions in the longitudinal direction of the space width were measured with a length-measurement SEM (CG-6300), manufactured by Hitachi High-Technologies Corporation. From the results, a tripled value (30) of a standard variation (σ) was determined as LWR. A smaller LWR value can yield a pattern with smaller roughness and a more uniform space width.

### [Limiting Resolution Evaluation]

A pattern was formed while gradually increasing the exposure dose from the optimum exposure dose at which the LS pattern can be formed, and in this event, the limit of the line width (nm) at which resolution is possible was determined using a length-measurement SEM (CG-6300), manufactured by Hitachi High-Technologies Corporation, to specify this value as limiting resolution (nm). A smaller value indicates that it is possible to form a finer pattern with better limiting resolution.

TABLE 4

| | | | | | | | Limiting |
|---|---|---|---|---|---|---|---|
| | Resist | PAB/PEB | | Pattern | EoP | LWR | resolution |
| | composition | (° C.) | Developer | type | (mJ/cm²) | (nm) | (nm) |
| Evaluation | R-01 | 110/90 | nBA | Positive | 35 | 3.0 | 13 |
| Example 2-1 | | | | | | | |
| Evaluation | R-02 | 110/90 | nBA | Positive | 36 | 3.2 | 13 |
| Example 2-2 | | | | | | | |
| Evaluation | R-03 | 110/90 | nBA | Positive | 35 | 3.2 | 13 |
| Example 2-3 | | | | | | | |
| Evaluation | R-04 | 110/90 | nBA | Positive | 35 | 3.0 | 13 |
| Example 2-4 | | | | | | | |
| Evaluation | R-05 | 110/90 | nBA | Positive | 35 | 3.0 | 13 |
| Example 2-5 | | | | | | | |
| Evaluation | R-06 | 110/90 | nBA | Positive | 34 | 3.1 | 13 |
| Example 2-6 | | | | | | | |
| Evaluation | R-07 | 110/90 | nBA | Positive | 35 | 3.3 | 14 |
| Example 2-7 | | | | | | | |
| Evaluation | R-08 | 110/90 | TMAH | Negative | 37 | 3.4 | 14 |
| Example 2-8 | | | | | | | |
| Evaluation | R-09 | 110/90 | TMAH | Negative | 37 | 3.3 | 14 |
| Example 2-9 | | | | | | | |
| Evaluation | R-10 | 110/90 | TMAH | Negative | 35 | 3.4 | 14 |
| Example 2-10 | | | | | | | |
| Evaluation | R-11 | 110/90 | TMAH | Negative | 35 | 3.3 | 14 |
| Example 2-11 | | | | | | | |
| Evaluation | R-12 | 110/90 | TMAH | Negative | 35 | 3.4 | 13 |
| Example 2-12 | | | | | | | |
| Evaluation | R-13 | 110/90 | TMAH | Negative | 33 | 3.2 | 13 |
| Example 2-13 | | | | | | | |
| Evaluation | R-14 | 110/90 | TMAH | Negative | 32 | 3.4 | 14 |
| Example 2-14 | | | | | | | |
| Evaluation | R-15 | 110/90 | TMAH | Negative | 31 | 3.4 | 14 |
| Example 2-15 | | | | | | | |
| Evaluation | R-16 | 110/90 | TMAH | Negative | 33 | 3.4 | 14 |
| Example 2-16 | | | | | | | |
| Evaluation | R-17 | 110/90 | TMAH | Negative | 34 | 3.4 | 14 |
| Example 2-17 | | | | | | | |
| Evaluation | R-18 | 110/90 | TMAH | Negative | 35 | 3.1 | 13 |
| Example 2-18 | | | | | | | |
| Evaluation | R-19 | 110/90 | nBA | Positive | 36 | 3.4 | 14 |
| Example 2-19 | | | | | | | |
| Evaluation | R-20 | 110/90 | nBA | Positive | 32 | 3.4 | 14 |
| Example 2-20 | | | | | | | |
| Evaluation | R-21 | 110/90 | nBA | Positive | 35 | 3.2 | 13 |
| Example 2-21 | | | | | | | |
| Comparative | CR-01 | 110/90 | nBA | Negative | 40 | 3.5 | 15 |
| Evaluation | | | | | | | |
| Example 2-1 | | | | | | | |
| Comparative | CR-02 | 105/90 | TMAH | Positive | 40 | 4.2 | 15 |
| Evaluation | | | | | | | |
| Example 2-2 | | | | | | | |
| Comparative | CR-03 | 105/90 | TMAH | Positive | 85 | 4.4 | 18 |
| Evaluation | | | | | | | |
| Example 2-3 | | | | | | | |
| Comparative | CR-04 | 105/90 | TMAH | Positive | 85 | 5.0 | 18 |
| Evaluation | | | | | | | |
| Example 2-4 | | | | | | | |

### Developers:

nBA (butyl acetate)
TMAH (2.38 mass % aqueous solution of
tetramethylammonium hydroxide)

From the results shown in Table 4, regarding Examples 2-1 to 2-21, where resist compositions containing heterocycle-containing hypervalent iodine compounds of the present invention were used, it was found that it was possible to form both positive and negative patterns depending on the developer used. In addition, comparing the inventive resist compositions with the resist compositions of Comparative Examples 2-1 and 2-2, it was found that excellent sensitivity, resolution, and LWR were achieved in Examples 2-1 to 2-21, and it was found that, even compared with the compositions of Comparative Examples 2-3 and 2-4, which were chemically amplified resist compositions using acid catalysis, excellent sensitivity, resolution, and LWR were achieved in Examples 2-1 to 2-21. In addition, from the results of Example 2-4, where resist composition R-04 was used, it can be seen that combinations of heterocycle-containing hypervalent iodine compounds of the present invention are also effective. Thus, it was found that the inventive resist composition was excellent in resolution in LS pattern formation by EUV exposure and so forth.

### [5] EUV Lithography Evaluation (Contact Hole Pattern)

### Examples 3-1 to 3-21 and Comparative Examples 3-1 to 3-4

Each of the resist compositions (R-01 to R-21 and CR-01 to CR-04) was applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness. Then, PAB was performed at the temperature shown in Table 5 for 60 seconds using a hot plate to produce a resist film with 50 nm in film thickness. Subsequently, the resist film was exposed using an EUV scanner NXE3400 (NA 0.33, σ 0.9/0.6, quadrupole illumination, 64 nm in pitch on wafer size, hole pattern mask with +20% bias), manufactured by ASML Holding N.V. Then, PEB was performed at the temperature shown in Table 5 for 60 seconds on a hot plate. Thereafter, development was performed with the developer shown in Table 5 for 30 seconds to obtain a hole pattern with 32 nm in size.

Regarding the obtained resist pattern, the following evaluations were carried out. The results are shown in Table 5.

### [Sensitivity Evaluation]

The contact hole pattern was observed using a length-measurement SEM (CG-6300), manufactured by Hitachi High-Technologies Corporation, and an optimum exposure dose Eₒₚ (mJ/cm²) to yield the hole pattern with a size of 32 nm was determined to specify this value as sensitivity.

### [CDU Evaluation]

Sizes of fifty hole patterns obtained by irradiation at the optimum exposure dose were measured, and a tripled value (30) of a standard variation (σ) calculated from the results was determined as CDU. A smaller CDU value can yield a pattern having a more uniform hole diameter.

### [Limiting Resolution Evaluation]

A hole pattern was formed while gradually decreasing the exposure dose from the optimum exposure dose at which the hole pattern can be formed, and in this event, the limit of the hole diameter (nm) at which resolution is possible was determined using a length-measurement SEM (CG-6300), manufactured by Hitachi High-Technologies Corporation, to specify this value as limiting resolution (nm). A smaller value indicates that it is possible to form a pattern having a finer hole diameter with better limiting resolution.

TABLE 5

| | | | | | | | Limiting |
|---|---|---|---|---|---|---|---|
| | Resist | PAB/PEB | | Pattern | EoP | CDU | resolution |
| | composition | (° C.) | Developer | type | (mJ/cm²) | (nm) | (nm) |
| Evaluation | R-01 | 110/90 | nBA | Positive | 30 | 2.7 | 25 |
| Example 3-1 | | | | | | | |
| Evaluation | R-02 | 110/90 | nBA | Positive | 35 | 2.1 | 24 |
| Example 3-2 | | | | | | | |
| Evaluation | R-03 | 110/90 | nBA | Positive | 36 | 2.1 | 24 |
| Example 3-3 | | | | | | | |
| Evaluation | R-04 | 110/90 | nBA | Positive | 33 | 2.1 | 24 |
| Example 3-4 | | | | | | | |
| Evaluation | R-05 | 110/90 | nBA | Positive | 35 | 2.2 | 25 |
| Example 3-5 | | | | | | | |
| Evaluation | R-06 | 110/90 | nBA | Positive | 36 | 2.3 | 25 |
| Example 3-6 | | | | | | | |
| Evaluation | R-07 | 110/90 | nBA | Positive | 32 | 2.4 | 26 |
| Example 3-7 | | | | | | | |
| Evaluation | R-08 | 110/90 | TMAH | Negative | 34 | 2.5 | 26 |
| Example 3-8 | | | | | | | |
| Evaluation | R-09 | 110/90 | TMAH | Negative | 34 | 2.4 | 26 |
| Example 3-9 | | | | | | | |
| Evaluation | R-10 | 110/90 | TMAH | Negative | 34 | 2.9 | 28 |
| Example 3-10 | | | | | | | |
| Evaluation | R-11 | 110/90 | TMAH | Negative | 34 | 2.4 | 26 |
| Example 3-11 | | | | | | | |
| Evaluation | R-12 | 110/90 | TMAH | Negative | 33 | 2.4 | 26 |
| Example 3-12 | | | | | | | |
| Evaluation | R-13 | 110/90 | TMAH | Negative | 33 | 2.2 | 26 |
| Example 3-13 | | | | | | | |
| Evaluation | R-14 | 110/90 | TMAH | Negative | 32 | 2.4 | 29 |
| Example 3-14 | | | | | | | |
| Evaluation | R-15 | 110/90 | TMAH | Negative | 32 | 2.9 | 29 |
| Example 3-15 | | | | | | | |
| Evaluation | R-16 | 110/90 | TMAH | Negative | 30 | 2.5 | 27 |
| Example 3-16 | | | | | | | |
| Evaluation | R-17 | 110/90 | TMAH | Negative | 30 | 2.5 | 25 |
| Example 3-17 | | | | | | | |
| Evaluation | R-18 | 110/90 | TMAH | Negative | 30 | 2.1 | 24 |
| Example 3-18 | | | | | | | |
| Evaluation | R-19 | 110/90 | nBA | Positive | 37 | 2.7 | 24 |
| Example 3-19 | | | | | | | |
| Evaluation | R-20 | 110/90 | nBA | Positive | 35 | 2.4 | 27 |
| Example 3-20 | | | | | | | |
| Evaluation | R-21 | 110/90 | nBA | Positive | 31 | 2.2 | 27 |
| Example 3-21 | | | | | | | |
| Comparative | CR-01 | 110/90 | nBA | Negative | 35 | 3.8 | 30 |
| Evaluation | | | | | | | |
| Example 3-1 | | | | | | | |
| Comparative | CR-02 | 105/90 | TMAH | Positive | 40 | 3.7 | 30 |
| Evaluation | | | | | | | |
| Example 3-2 | | | | | | | |
| Comparative | CR-03 | 105/90 | TMAH | Positive | 50 | 4.2 | 32 |
| Evaluation | | | | | | | |
| Example 3-3 | | | | | | | |
| Comparative | CR-04 | 105/90 | TMAH | Positive | 50 | 4.2 | 32 |
| Evaluation | | | | | | | |
| Example 3-4 | | | | | | | |

From the results shown in Table 5, regarding Examples 3-1 to 3-21, where resist compositions containing heterocycle-containing hypervalent iodine compounds of the present invention were used, it was found that it was possible to form both positive and negative patterns depending on the choice of the developer used. In addition, comparing the inventive resist compositions with the resist compositions of Comparative Evaluation Examples 3-1 and 3-2, it was found that excellent sensitivity, resolution, and CDU were achieved in Examples 3-1 to 3-21, and it was found that, even compared with the compositions of Comparative Evaluation Examples 3-3 and 3-4, which were chemically amplified resist compositions using acid catalysis, excellent sensitivity, resolution, and CDU were achieved in Examples 3-1 to 3-21. In addition, from the results of Example 3-4, where resist composition R-04 was used, it can be seen that combinations of heterocycle-containing hypervalent iodine compounds of the present invention are also effective. Thus, it was found that the inventive resist composition was excellent in resolution in contact hole pattern formation by EUV exposure and so forth.

The present description includes the following embodiments.

[1]: A resist composition comprising: at least one kind of hypervalent iodine compound selected from the following formulae (1), (2), and (3); a carboxy-group-containing compound; and a solvent,

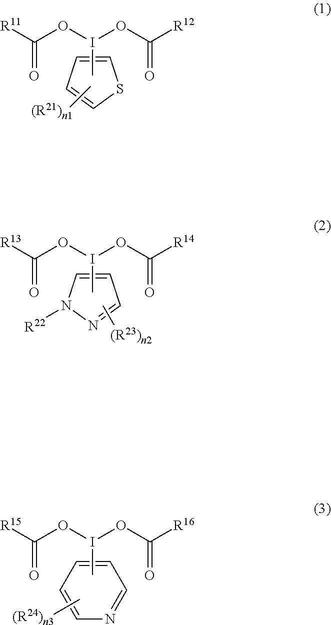

wherein “n1” represents 0, 1, 2, or 3, “n2” represents 0, 1, or 2, “n3” represents 0, 1, 2, 3, or 4, R¹¹ to R¹⁶ each independently represent a halogen atom or a hydrocarbyl group having 1 to 10 carbon atoms and optionally containing a heteroatom, and R²¹ to R²⁴ each independently represent a halogen atom or a hydrocarbyl group having 1 to 40 carbon atoms and optionally containing a heteroatom, when “n1” is 2 or more, the R²¹s are identical to or different from each other and the R²¹s may be bonded to each other to form a ring together with the carbon atoms of the heteroaromatic ring bonded thereto, when “n2” is 2, the R²³s are identical to or different from each other and the R²³s may be bonded to each other to form a ring together with the carbon atoms of the heteroaromatic ring bonded thereto, and when “n3” is 2 or more, the R²⁴s are identical to or different from each other and the R²⁴s may be bonded to each other to form a ring together with the carbon atoms of the heteroaromatic ring bonded thereto.

[2]: The resist composition according to [1], wherein the carboxy-group-containing compound is one or both of a polymer including a repeating unit represented by the following formula (4) and a compound represented by the following formula (5),

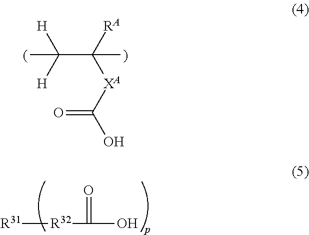

wherein R^{A} represents a hydrogen atom, a halogen atom, a methyl group, or a trifluoromethyl group; X^{A} represents a single bond, a phenylene group, a naphthylene group, or *—C(═O)—O—X^{A1}—; X^{A1} represents a saturated hydrocarbylene group having 1 to 10 carbon atoms, a phenylene group, or a naphthylene group, the saturated hydrocarbylene group optionally containing a hydroxy group, an ether bond, an ester bond, or a lactone ring; “*” represents an attachment point to the carbon atom of the main chain; “p” represents 1, 2, 3, or 4; R³¹ represents a p-valent hydrocarbon group having 1 to 40 carbon atoms or a p-valent heterocyclic group having 2 to 40 carbon atoms, when “p” is 2, the R³¹ may be an ether bond, a carbonyl group, an azo group, a thioether bond, a carbonate bond, a carbamate bond, a sulfinyl group, or a sulfonyl group, wherein part or all of hydrogen atoms of the p-valent hydrocarbon group or the p-valent heterocyclic group may be substituted with a group containing a heteroatom, and part of —CH₂— of the p-valent hydrocarbon group may be substituted with a group containing a heteroatom; and R³² represents a single bond or a hydrocarbylene group having 1 to 20 carbon atoms, part or all of hydrogen atoms of the hydrocarbylene group may be substituted with a group containing a heteroatom, part of —CH₂— of the hydrocarbylene group may be substituted with a group containing a heteroatom, and when “p” is 2, 3, or 4, the R³²s are identical to or different from each other.

[3]: A laminate comprising: a substrate; and a resist film, which is a film body formed of the resist composition according to [1] or [2], on the substrate.

[4]: The laminate according to [3], further comprising a resist underlayer film between the substrate and the resist film.

[5]: The laminate according to [3] or [4], wherein the resist film contains a product made by a ligand exchange reaction of the hypervalent iodine compound and the carboxy-group-containing compound.

[6]: A patterning process comprising the steps of:
forming a resist film by using the resist composition according to [1] or [2] on a substrate or on a resist underlayer film of a substrate on which the resist underlayer film has been laminated;
exposing the resist film by using a high-energy beam; and
developing the exposed resist film by using a developer.

[7]: The patterning process according to [6], wherein the high-energy beam is an i-line, a KrF excimer laser beam, an ArF excimer laser beam, an electron beam, or an extreme ultraviolet ray.

[8]: The patterning process according to [6] or [7], wherein the developer dissolves exposed portions and does not dissolve unexposed portions.

[9]: The patterning process according to [6] or [7], wherein the developer dissolves unexposed portions and does not dissolve exposed portions.

It should be noted that the present invention is not limited to the above-described embodiments. The embodiments are just examples, and any examples that have substantially the same feature and demonstrate the same functions and effects as those in the technical concept disclosed in claims of the present invention are included in the technical scope of the present invention.

1. A resist composition comprising: at least one kind of hypervalent iodine compound selected from the following formulae (1), (2), and (3); a carboxy-group-containing compound; and a solvent, 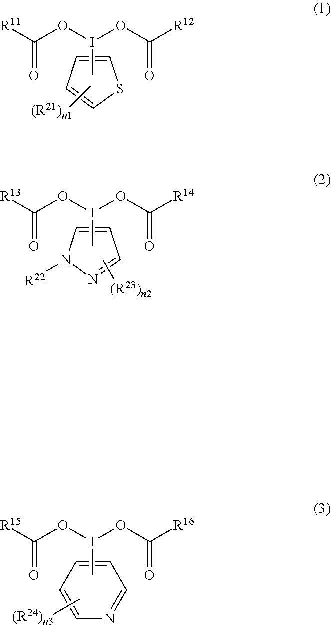
wherein “n1” represents 0, 1, 2, or 3, “n2” represents 0, 1, or 2, “n3” represents 0, 1, 2, 3, or 4, R¹¹ to R¹⁶ each independently represent a halogen atom or a hydrocarbyl group having 1 to 10 carbon atoms and optionally containing a heteroatom, and R²¹ to R²⁴ each independently represent a halogen atom or a hydrocarbyl group having 1 to 40 carbon atoms and optionally containing a heteroatom, when “n1” is 2 or more, the R²¹s are identical to or different from each other and the R²¹s may be bonded to each other to form a ring together with the carbon atoms of the heteroaromatic ring bonded thereto, when “n2” is 2, the R²³s are identical to or different from each other and the R²³s may be bonded to each other to form a ring together with the carbon atoms of the heteroaromatic ring bonded thereto, and when “n3” is 2 or more, the R²⁴s are identical to or different from each other and the R²⁴s may be bonded to each other to form a ring together with the carbon atoms of the heteroaromatic ring bonded thereto.

2. The resist composition according to claim 1, wherein the carboxy-group-containing compound is one or both of a polymer including a repeating unit represented by the following formula (4) and a compound represented by the following formula (5), 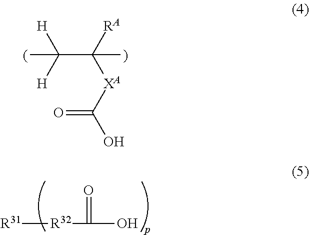
wherein R^{A} represents a hydrogen atom, a halogen atom, a methyl group, or a trifluoromethyl group; X^{A} represents a single bond, a phenylene group, a naphthylene group, or *—C(═O)—O—X^{A1}—; X^{A1} represents a saturated hydrocarbylene group having 1 to 10 carbon atoms, a phenylene group, or a naphthylene group, the saturated hydrocarbylene group optionally containing a hydroxy group, an ether bond, an ester bond, or a lactone ring; “*” represents an attachment point to the carbon atom of the main chain; “p” represents 1, 2, 3, or 4; R³¹ represents a p-valent hydrocarbon group having 1 to 40 carbon atoms or a p-valent heterocyclic group having 2 to 40 carbon atoms, when “p” is 2, the R³¹ may be an ether bond, a carbonyl group, an azo group, a thioether bond, a carbonate bond, a carbamate bond, a sulfinyl group, or a sulfonyl group, wherein part or all of hydrogen atoms of the p-valent hydrocarbon group or the p-valent heterocyclic group may be substituted with a group containing a heteroatom, and part of —CH₂— of the p-valent hydrocarbon group may be substituted with a group containing a heteroatom; and R³² represents a single bond or a hydrocarbylene group having 1 to 20 carbon atoms, part or all of hydrogen atoms of the hydrocarbylene group may be substituted with a group containing a heteroatom, part of —CH₂— of the hydrocarbylene group may be substituted with a group containing a heteroatom, and when “p” is 2, 3, or 4, the R³²s are identical to or different from each other.

3. A laminate comprising: a substrate; and a resist film, which is a film body formed of the resist composition according to claim 1, on the substrate.

4. A laminate comprising: a substrate; and a resist film, which is a film body formed of the resist composition according to claim 2, on the substrate.

5. The laminate according to claim 3, further comprising a resist underlayer film between the substrate and the resist film.

6. The laminate according to claim 4, further comprising a resist underlayer film between the substrate and the resist film.

7. The laminate according to claim 3, wherein the resist film contains a product made by a ligand exchange reaction of the hypervalent iodine compound and the carboxy-group-containing compound.

8. The laminate according to claim 4, wherein the resist film contains a product made by a ligand exchange reaction of the hypervalent iodine compound and the carboxy-group-containing compound.

9. A patterning process comprising the steps of:
forming a resist film by using the resist composition according to claim 1 on a substrate or on a resist underlayer film of a substrate on which the resist underlayer film has been laminated;
exposing the resist film by using a high-energy beam; and
developing the exposed resist film by using a developer.

10. A patterning process comprising the steps of:
forming a resist film by using the resist composition according to claim 2 on a substrate or on a resist underlayer film of a substrate on which the resist underlayer film has been laminated;
exposing the resist film by using a high-energy beam; and
developing the exposed resist film by using a developer.

11. The patterning process according to claim 9, wherein the high-energy beam is an i-line, a KrF excimer laser beam, an ArF excimer laser beam, an electron beam, or an extreme ultraviolet ray.

12. The patterning process according to claim 10, wherein the high-energy beam is an i-line, a KrF excimer laser beam, an ArF excimer laser beam, an electron beam, or an extreme ultraviolet ray.

13. The patterning process according to claim 9, wherein the developer dissolves exposed portions and does not dissolve unexposed portions.

14. The patterning process according to claim 11, wherein the developer dissolves exposed portions and does not dissolve unexposed portions.

15. The patterning process according to claim 9, wherein the developer dissolves unexposed portions and does not dissolve exposed portions.

16. The patterning process according to claim 11, wherein the developer dissolves unexposed portions and does not dissolve exposed portions.
